SEQUENCE LISTING (25 sequences)

SEQ ID NO: 1 - protein, 114 aa, Synthetic construct
QVQLQQPGAELARPGAAVKLSCKASGYTFTNYWMNWVKQRPGQGLEWIGMIDPSDNKTLYNPMFKDKATLTVDKSSSTAYMQLSSLTSEDSAVYYCARRGFAYWGQGTLVTVSA

SEQ ID NO: 2 - protein, 104 aa, Synthetic construct
MTQTPSSLSASLGDTITITCHASQNINVWLSWYQQKPGNIPKVLIYKASNLHTGVPSRFSGSGSGTGFTLTISSLQPEDIATYYCQQGQSYPLTFGGGTKLEIK

SEQ ID NO: 3 - protein, 5 aa, Synthetic construct
NYWMN

SEQ ID NO: 4 - protein, 8 aa, Synthetic construct
MIDPSDNK

SEQ ID NO: 5 - protein, 5 aa, Synthetic construct
RGFAY

SEQ ID NO: 6 - protein, 11 aa, Synthetic construct
HASQNINVWLS

SEQ ID NO: 7 - protein, 7 aa, Synthetic construct
KASNLHT

SEQ ID NO: 8 - protein, 9 aa, Synthetic construct
QQGQSYPLT

SEQ ID NO: 9 - protein, 338 aa, Synthetic construct
CAGGTCCAACTGCAGCAGCCTGGGGCTGAGCTGGCGAGGCCTGGGGCTGCAGTGAAGCTGTCCTGCAAGGCTTCTGGCTACACCTTCACCAACTACTGGATGAACTGGGTGAAGCAGAGGCCTGGACAAGGCCTTGAATGGATTGGTATGATTGATCCTTCAGACAATAAAACTCTCTACAATCCAATGTTCAAGGACAAGGCCACATTGACTGTAGACAAATCCTCCAGCACAGCCTACATGCAGCTCAGCAGTCTGACATCTGAGGACTCTGCGGTCTATTACTGTGCAAGACGAGGCTTTGCTTACTGGGGCCAAGGGACTCTGGTCACTGTAAG

SEQ ID NO: 10 - protein, 312 aa, Synthetic construct
ATGACCCAGACTCCATCCAGTCTGTCTGCATCCCTTGGAGACACAATTACCATCACTTGCCATGCCAGTCAGAACATTAATGTTTGGTTAAGCTGGTACCAGCAGAAACCAGGAAATATTCCTAAAGTATTGATCTATAAGGCCTCCAATTTGCACACAGGCGTCCCATCAAGGTTTAGTGGCAGTGGATCTGGAACAGGTTTCACATTAACCATCAGCAGCCTGCAGCCTGAAGACATTGCCACTTACTACTGTCAACAGGGTCAAAGTTATCCTCTGACGTTTGGTGGAGGCACCAAACTGGAAATCAAA

SEQ ID NO: 11 - protein, 113 aa, Synthetic construct
VQLQESGPELVKPGASVKMSCKASGYTFTDYYMNWVKQSHGKSLEWIGDINPYNGGTSYNQKFKGKATLTVDKSSRTAYMQLNSLTSEDSAVYYCARGGSPQWGQGTTLTVSS

SEQ ID NO: 12 - protein, 112 aa, Synthetic construct
DIVMTQTPLTLSVTIGQPASISCKSSQSLLHSDGKTYLNWLLQRPGQSPKRLIYLVSKLDSGVPDRFTGSGSGTDFTLKISRVEAEDLGVYYCWQGTHFPWTFGGGTKLEIK

SEQ ID NO: 13 - protein, 133 aa, Synthetic construct
MGWSCIILFLVATATGVHSQVQLQQPGAELVRPGAAVKLSCKASGYTFTSYWMNWVKQRPGQGLEWIGMIDPSDSKTHYNQMFKDKATLTVDKSSSTAYMQLSSLTFEDSAVYYCARRGFPYWGQGTLVTVSA

SEQ ID NO: 14 - protein, 127 aa, Synthetic construct
MRVLAELLGLLLFCFLGVRCDIQMNQSPSSLSASLGDTITITCHASQNINVWLSWYQQKPGNIPKVLIYKASNLHTGVPSRFSGSGSGTGFTLTISSLQPEDIATYYCQQGQSYPLTFGGGTKLEIK

SEQ ID NO: 15 - protein, 8 aa, Synthetic construct
DYKDDDDK

SEQ ID NO: 16 - protein, 6 aa, Synthetic construct
HHHHHH

SEQ ID NO: 17 - protein, 9 aa, Synthetic construct
YPYDVPDYA

SEQ ID NO: 18 - protein, 45 aa, Synthetic construct
TTTPAPRPPTPAPTIASQPLSLRPEACRPAAGGAVHTRGLDFACD

SEQ ID NO: 19 - protein, 24 aa, Synthetic construct
IYIWAPLAGTCGVLLLSLVITLYC

SEQ ID NO: 20 - protein, 42 aa, Synthetic construct
KRGRKKLLYIFKQPFMRPVQTTQEEDGCSCRFPEEEEGGCEL

SEQ ID NO: 21 - protein, 15 aa, Synthetic construct
GGGGSGGGGSGGGGS

SEQ ID NO: 22 - protein, 6 aa, Synthetic construct
DITLSQ

SEQ ID NO: 23 - protein, 310 aa, Synthetic construct
MRRAALWLWLCALALSLQPALPQIVATNLPPEDQDGSGDDSDNFSGSGAGALQDITLSQQTPSTWKDTQLLTAIPTSPEPTGLEATAASTSTLPAGEGPKEGEAVVLPEVEPGLTAREQEATPRPRETTQLPTTHLASTTTATTAQEPATSHPHRDMQPGHHETSTPAGPSQADLHTPHTEDGGPSATERAAEDGASSQLPAAEGSGEQDFTFETSGENTAVVAVEPDRRNQSPVDQGATGASQGLLDRKEVLGGVIAGGLVGLIFAVCLVGFMLYRMKKKDEGSYSLEEPKQANGGAYQKPTKQEEFYA

SEQ ID NO: 24 - protein, 311 aa, Synthetic construct
MRRAALWLWLCALALRLQPALPQIVAVNVPPEDQDGSGDDSDNFSGSGTGALPDTLSRQTPSTWKDVWLLTATPTAPEPTSSNTETAFTSVLPAGEKPEEGEPVLHVEAEPGFTARDKEKEVTTRPRETVQLPITQRASTVRVTTAQAAVTSHPHGGMQPGLHETSAPTAPGQPDHQPPRVEGGGTSVIKEVVEDGTANQLPAGEGSGEQDFTFETSGENTAVAAVEPGLRNQPPVDEGATGASQSLLDRKEVLGGVIAGGLVGLIFAVCLVAFMLYRMKKKDEGSYSLEEPKQANGGAYQKPTKQEEFYA

SEQ ID NO: 25 - protein, 4 aa, Synthetic construct
DFTF

(19) United States
(12) Patent Application Publication
(10) Pub. No.: US 2026/0131036 A1
(43) Pub. Date: May 14, 2026

(54) CD138/SYNDECAN1 ANTIBODIES AND METHODS OF USE THEREOF

(71) Applicant: BOARD OF REGENTS, THE UNIVERSITY OF TEXAS SYSTEM, Austin, TX (US)

(72) Inventors: Wantong YAO, Houston, TX (US); Haoqiang YING, Houston, TX (US); Zecheng YANG, Houston, TX (US); Laura BOVER, Pearland, TX (US); Henry Charles MANNING, Houston, TX (US); Aiko YAMAGUCHI, Houston, TX (US); Ryan Patrick COLL, Houston, TX (US)

(21) Appl. No.: 19/399,147

(22) Filed: Nov. 24, 2025

Related U.S. Application Data
(63) Continuation-in-part of application No. PCT/US2024/030870, filed on May 23, 2024.
(60) Provisional application No. 63/503,785, filed on May 23, 2023.
(60) Provisional application No. 63/725,986, filed on Nov. 27, 2024.

(51) Int. Cl. A61K 51/10; A61K 40/31; A61K 40/42; A61K 49/00; A61P 35/00; C07K 14/725; C07K 16/28; G01N 33/575

(52) U.S. Cl. CPC A61K 51/1096; A61K 40/31; A61K 40/4224; A61K 49/0058; A61K 51/1027; A61P 35/00; C07K 14/7051; C07K 16/2896; G01N 33/5759; A61K 2121/00; A61K 2123/00; C07K 2317/732; C07K 2317/77; C07K 2317/92; C07K 2319/02; C07K 2319/03

(57) ABSTRACT

Provided herein are antibodies and antigen binding portions thereof, as well as theranostic pairs of radioimmunoconjugates, that specifically bind to Syndecan-1 (SDC1, CD138); various compositions of such antibodies, antigen binding portions thereof, and theranostic pairs of radioimmunoconjugates; and recombinant nucleic acids encoding the antibodies or antigen binding portions thereof. Also provided are methods of using the antibodies or antigen binding portions thereof and theranostic pairs in cancer therapeutics and diagnostics.

### CROSS REFERENCE TO RELATED APPLICATION

This application claims priority to and the benefit of International Patent Application No. PCT/US2024/030870, filed May 23, 2024, which claims priority to and the benefit of U.S. Provisional Patent Application Ser. No. 63/503,785, filed May 23, 2023. This application claims priority to and the benefit of U.S. Provisional Patent Application Ser. No. 63/725,986, filed Nov. 27, 2024. The contents of each of these applications are incorporated herein by this reference as if fully set forth herein.

### REFERENCE TO A SEQUENCE LISTING SUBMITTED AS XML VIA EFS-WEB

The instant application contains a Sequence Listing in XML format. The Sequence Listing, named 090723_1533105_seqlist.xml was created on Nov. 20, 2025, is 24,576 bytes in size, is part of the specification, and is hereby incorporated by reference in its entirety.

### BACKGROUND

Kras is one of the most frequently mutated oncogenes in human cancer, with amino acid glycine 12 as the most common mutation site. Mutant KRAS (mKRAS) is critical for disease initiation in numerous cancer types, such as pancreatic adenocarcinoma (PDAC) and non-small cell lung cancer (NSCLC), and accordingly, is detectable in early neoplastic lesions and remains functional in invasive metastatic disease. Constitutive mKRAS signaling drives uncontrolled proliferation and enhances cancer cell survival through the activation of downstream signaling pathways, such as the RAF-mitogen-activated kinase (MAPK), phosphoinositide-3-kinase (PI3K), and RALGDS pathways. Thus, due to its high prevalence in cancer types and central role in tumor progression, the effective and specific targeting of mKRAS has been a continuing priority in anti-cancer drug development.

PDAC is a highly aggressive malignancy with an overall five-year survival rate of 12%. Greater than 50% of patients present with distant metastatic dissemination at diagnosis, most commonly in the liver, lung, and peritoneum, further limiting the five-year survival to 3.2% in these patients. Current treatment options for PDAC include tissue agnostic chemotherapeutic combinations, which are largely ineffective and fraught with side effects. Further, clinical trials of certain immunotherapy strategies have largely been unsuccessful in the treatment of PDAC due to a highly immunosuppressive microenvironment. Thus, there remains a clinical need for effective treatment modalities for patients with PDAC.

### SUMMARY

The Summary is provided to introduce a selection of concepts that are further described below in the Detailed Description. This Summary is not intended to identify key or essential features of the claimed subject matter, nor is it intended to be used as an aid in limiting the scope of the claimed subject matter.

In one aspect, provided herein are isolated antibodies or antibody fragments thereof that specifically bind to syndecan 1 (SDC1). In some embodiments, the isolated antibody or antibody fragment thereof is a non-fucosylated monoclonal antibody or antibody fragment. In some embodiments, the isolated antibody or antibody fragment binds SEQ ID NO: 22. In some embodiments, the isolated antibody or antibody fragment comprises a heavy chain variable region comprising a CDRH1 comprising SEQ ID NO: 3, a CDRH2 comprising SEQ ID NO: 4; and a CDRH3 comprising SEQ ID NO: 5; and a light chain variable region comprising a CDRL1 comprising SEQ ID NO: 6: a CDRL2 comprising SEQ ID NO: 7; and a CDRL3 comprising SEQ ID NO: 8. In some embodiments, the isolated antibody or antibody fragment comprises a heavy chain variable region (VH) having at least 90% identity to SEQ ID NO: 1; and a light chain variable region (VL) having at least 90% identity to SEQ ID NO: 2. In some embodiments, the isolated antibody or antibody fragment comprises a light chain variable sequence as set forth in SEQ ID NO: 2. In some embodiments, the isolated antibody or antibody fragment thereof comprises a heavy chain variable sequence as set forth in SEQ ID NO: 1. In some embodiments, the isolated antibody or antibody fragment thereof comprises a heavy chain variable sequence as set forth in SEQ ID NO: 1 and a light chain variable sequence as set forth in SEQ ID NO: 2.

In some embodiments, the isolated antibody or antibody fragment thereof is a non-fucosylated antibody or antibody fragment. In some embodiments, the isolated antibody or antibody fragment thereof is a monovalent scFv (single chain fragment variable) antibody, divalent scFv, Fab fragment, F(ab)₂ fragment, F(ab)₃ fragment, Fv fragment, nanobody, or single chain antibody. In some embodiments, the isolated antibody or antibody fragment thereof is a chimeric antibody, bispecific antibody, trispecific or multispecific antibody, or BiTE. In some embodiments, the isolated antibody or antibody fragment thereof is an IgG antibody or a recombinant IgG antibody or antibody fragment. In some embodiments, the isolated antibody is a bispecific antibody that specifically binds SDC1 and PD1 or that specifically binds SDC1 and 4-1BB.

In some embodiments, the isolated antibody or antibody fragment is conjugated or fused to an imaging agent, a cytotoxic agent, a metal, or a radioactive moiety. In some embodiments, the antibody or antibody fragment thereof is conjugated to an imaging agent wherein the imaging agent is a fluorophore. In some embodiments, the antibody or antibody fragment thereof is conjugated or fused to a radioactive moiety wherein the radioactive moiety is ¹⁶¹Tb, ²²⁵Ac, ¹⁶¹Tb/²²⁵Ac, ⁸⁹Zr, ¹⁷⁷Lu, ¹³⁴Ce, ¹⁴⁰Nd, ¹⁶⁹Er, ¹³⁴Ce/¹³⁴La, or ¹⁴⁰Nd/¹⁴⁰Pr. In some embodiments, the antibodies or antibody fragments are immune conjugates. In some embodiments, the antibodies or antibody fragments are conjugated to flagellin or a flagellin derivative. In some embodiments, the isolated antibodies or antibody fragments are antibody-drug conjugates.

In another aspect, pharmaceutical compositions are provided. In some embodiments, the pharmaceutical composition comprises an isolated antibody or antibody fragment thereof that specifically binds SDC1 and a pharmaceutically acceptable carrier. In some embodiments, the isolated antibody or antibody fragment is conjugated or fused to a cytotoxic agent, a metal, a radioactive moiety, or a drug.

In another aspect, provided herein are isolated nucleic acids encoding the antibody heavy and/or light chain variable regions of the isolated antibody of any of the disclosed embodiments. In some embodiments, the nucleic acids comprise a nucleotide sequence that is at least 90% identical to SEQ ID NO: 9. In some embodiments, the nucleic acids comprise a nucleotide sequence that is at least 90% identical to SEQ ID NO: 10.

In another aspect, provided herein are expression vectors comprising the nucleic acids of any one of the disclosed embodiments.

In another aspect, provided herein are hybridomas or engineered cells comprising a nucleic acid encoding an antibody or antibody fragment of any one of the disclosed embodiments.

In another aspect, provided herein are methods of making antibodies or antibody fragments of any one of the disclosed embodiments. In some embodiments, the methods comprise culturing a hybridoma or engineered cell comprising a nucleic acid encoding any antibody or antibody fragment disclosed herein under conditions that allow expression of the antibody or antibody fragment, and optionally isolating the antibody or antibody fragment from the culture.

In a further aspect, provided herein are chimeric antigen receptor (CAR) proteins comprising an antigen binding domain comprising a heavy chain variable region (VH) comprising VHCDR1, VHCDR2, and VHCDR3 amino acid sequences from any isolated antibody or antibody fragment disclosed herein; and a light chain variable region (VL) comprising VLCDR1, VLCDR2, and VLCDR3 amino acid sequences from any isolated antibody or antibody fragment disclosed herein. In some embodiments, the antigen binding domain comprises heavy and light chain CDR sequences as follows: a heavy chain variable region (VH) having at least 90% identity to SEQ ID NO: 1 and comprising a CDRH1 amino acid sequence comprising SEQ ID NO: 3, a CDRH2 amino acid sequence comprising SEQ ID NO: 4, and a CDRH3 amino acid sequence comprising SEQ ID NO: 5; and a light chain variable region (VL) having at least 90% identity to SEQ ID NO: 2 and comprising a CDRL1 amino acid sequence comprising SEQ ID NO: 6, a CDRL2 amino acid sequence comprising SEQ ID NO: 7, and a CDRL3 amino acid sequence comprising SEQ ID NO: 8. In some embodiments, the antigen binding domain comprises a heavy chain variable region (VH) comprising CDRH1, CDRH2, and CDRH3 amino acid sequences derived from SEQ ID NO: 1; and a light chain variable region (VL) comprising CDRH1, CDRH2, and CDRH3 amino acid sequences derived from SEQ ID NO: 2. In some embodiments, the antigen binding domain comprises a heavy chain variable sequence having a sequence set forth in SEQ ID NO: 1 and a light chain variable sequence having a sequence set forth in SEQ ID NO: 2. In some embodiments, the antigen binding domain specifically binds Syndecan-1 (SDC1).

In some embodiments, the chimeric antigen receptor further comprises a hinge domain, a transmembrane domain, and an intracellular signaling domain. In some embodiments, the chimeric antigen receptor further comprises a hinge domain, wherein the hinge domain is a CD8a hinge domain or an IgG4 hinge domain. In some embodiments, the chimeric antigen receptor further comprises a hinge domain, wherein the hinge domain is a CD8a transmembrane domain or an CD28 transmembrane domain. In some embodiments, the chimeric antigen receptor comprises an intracellular signaling domain, wherein the intracellular signaling domain comprises a CD3% intracellular signaling domain.

In another aspect, provided herein are nucleic acid molecules encoding a CAR of any of the disclosed embodiments. In some embodiments, the sequence encoding the CAR is operatively linked to an expression control sequence. In another aspect, expression vectors are provided that comprise a nucleic acid molecule encoding a CAR of any of the embodiments disclosed herein.

In some aspects, provided herein are engineered cells comprising a nucleic acid molecule encoding a CAR of any one of the disclosed embodiments. In some embodiments, the cell is a T cell. In some embodiments, the cell is an NK cell. In some embodiments, the nucleic acid is integrated into a genome of the cell. In some embodiments, the cell is a human cell. In another aspect, provided herein are pharmaceutical compositions comprising a population of the engineered cells as disclosed herein and a pharmaceutically acceptable carrier.

In a further aspect, provided herein are methods of treating cancer in a patient. In some embodiments, the methods comprise administering to the patient an anti-tumor effective amount of the pharmaceutical composition of any one of the disclosed embodiments. In some embodiments, the pharmaceutical composition comprises a population of cells, wherein the cells are allogeneic cells. In some embodiments, the pharmaceutical composition comprises a population of cells, wherein the cells are autologous cells. In some embodiments, the pharmaceutical composition comprises a population of cells, wherein the cells are HLA matched to the patient. In some embodiments, the pharmaceutical composition comprises an isolated antibody or antibody as disclosed herein conjugated to a therapeutic agent. In some embodiments, the therapeutic agent is at least one of a cytotoxicity agent, a chemotherapeutic agent, or an immunosuppressive agent. In some embodiments, the therapeutic agent is a moiety that specifically binds to an immune cell. In some embodiments, the immune cell is a T cell. In some embodiments, the immune cell is a natural killer cell. In some embodiments, the cancer has been determined to express an elevated level of SDC1 relative to a healthy tissue. In some embodiments, the cancer is pancreatic cancer, colorectal cancer, or non-small lung cell cancer. In some embodiments, the administration of the pharmaceutical composition reduces macropinocytosis in the patient. In some embodiments, the patient has previously failed to respond to an immune checkpoint inhibitor. In some embodiments, the patient has failed to respond to a Kras targeted therapy. In some embodiments, the patient has relapsed.

In some embodiments, the method further comprises administering at least a second anti-cancer therapy. In some embodiments, the second anti-cancer therapy is a chemotherapy, molecular targeted therapy, immunotherapy, radiotherapy, radioimmunotherapy, phototherapy, gene therapy, surgery, hormonal therapy, epigenetic modulation, anti-angiogenic therapy, or cytokine therapy. In some embodiments, the second anti-cancer therapy is selected from a group consisting of a PD1 antibody, a 4-1BB antibody, gemcitabine, AMG510, MRTX1133, or a combination thereof.

In another aspect, provided herein are methods of detecting the presence of SDC1 in a biological sample. In some embodiments, the methods comprise contacting a biological sample with the isolated antibody or antibody fragment thereof of any one of the disclosed embodiments, and detecting an amount of binding of the isolated antibody or antibody fragment thereof as a determination of the presence of SDC1 in the biological sample. In some embodiments, the biological sample comprises cancer cells. In some embodiments, the biological sample comprises a sample from a tumor from a patient.

In another aspect, provided herein are methods of imaging a tumor in a patient with an SDC1 expressing cancer. In some embodiments, the method comprises administering to the patient an isolated antibody or antibody fragment of any one of the disclosed embodiments conjugated to an imaging label and detecting the imaging label in the patient to obtain an image of the tumor.

In another aspect, provided herein are methods of monitoring the response of a patient with an SDC1 expressing cancer to cancer therapy. In some embodiments, the method comprises administering to the patient the isolated antibody or antibody fragment thereof of any one of the disclosed embodiments conjugated to an imaging label at a first time point before the patient receives cancer therapy, detecting the imaging label in the patient to obtain a first image of a tumor, administering to the patient an isolated antibody or antibody fragment thereof in accordance with any one of the disclosed embodiments conjugated to an imaging agent at a second time point after the patient receives cancer therapy, detecting the imaging label in the patient at a second time point after the patient received cancer therapy, and comparing the first image to the second image to determine whether a change in tumor size has occurred. In some embodiments, the method comprises repeating the steps of administering, detecting, and comparing at a third time point after the patient receives cancer therapy. In some embodiments, the imaging label comprises a radioisotope, a bioluminescent label, a chemiluminescent label, or a paramagnetic compound.

In another aspect, provided herein is a method of assessing the likelihood of responsiveness of a patient with cancer to treatment with an SDC1 targeted therapy. In some embodiments, the method comprises measuring in a tumor sample from a patient an amount of expression of SDC1, and determining if the patient has a cancer characterized as having a high level of SDC1. In some embodiments, the amount of SDC1 expression in the tumor sample is measured using an isolated antibody or antibody fragment thereof disclosed herein. In some embodiments, the SDC1 targeted therapy comprises administration of the pharmaceutical composition of any one of the disclosed embodiments.

In another aspect, a theranostic pair is provided. In some embodiments, the theranostic pair comprises a first radiolabeled antibody comprising a syndecan 1 (SDC1) antibody or antibody fragment thereof conjugated to a first chelating agent and a first radioactive moiety that is an imaging agent; and a second radiolabeled antibody comprising the SDC1 antibody or antibody fragment thereof conjugated to a second chelating agent and a second radioactive moiety that is a therapeutic agent. In some embodiments, the SDC1 antibody or antibody fragment comprises a heavy chain variable region comprising a CDRH1 comprising SEQ ID NO: 3: a CDRH2 comprising SEQ ID NO: 4; and a CDRH3 comprising SEQ ID NO: 5; and a light chain variable region comprising a CDRL1 comprising SEQ ID NO: 6: a CDRL2 comprising SEQ ID NO: 7; and a CDRL3 comprising SEQ ID NO: 8. In some embodiments, the first radioactive moiety is selected from a group consisting of ⁸⁹Zr, ¹³¹I, ¹²³I, ¹¹¹I, ^{99m}Tc, ⁹⁰Y, ¹⁸⁶Re, ¹⁸⁸Re, ³²P, ¹⁵³Sm, ⁶⁷Ga, ²⁰¹Tl, ⁷⁷Br, or ¹⁸F. In certain embodiments, the first radioactive moiety is 89Zr. In some embodiments, the second radioactive moiety is selected from a group consisting of ¹⁶¹Tb, ²²⁵Ac, ¹⁶¹Tb/²²⁵Ac, ¹⁷⁷Lu, ¹³⁴Ce, ¹⁴⁰Nd, ¹⁶⁹Er, ¹³⁴Ce/¹³⁴La, and ¹⁴⁰Nd/¹⁴⁰Pr. In certain embodiments, the second radioactive moiety is ¹⁶¹Tb. In some embodiments, the SDC1 antibody or antibody fragment comprises a heavy chain variable region (VH) having at least 90% identity to SEQ ID NO: 1; and a light chain variable region (VL) having at least 90% identity to SEQ ID NO: 2. In some embodiments, the SDC1 antibody or antibody fragment comprises a heavy chain variable region (VH) comprising SEQ ID NO: 1; and a light chain variable region (VL) comprising SEQ ID NO: 2. In some embodiments, the chelating agent comprises deferoxamine (DFO), 1,4,7,10-tetraazacyclododecane (DO3A), or diethylenetriaminepentaacetic acid (DTPA). In some embodiments, the first radiolabeled antibody comprises the SDC1 antibody or antibody fragment thereof conjugated to DFO and 89Zr, and wherein the second radiolabeled antibody comprises the SDC1 antibody or antibody fragment thereof conjugated to DO3A and ¹⁶¹Tb. In some embodiments, the theranostic pair comprises a third radiolabeled antibody comprising the SDC1 antibody or antibody fragment thereof conjugated to a third chelating agent and a third radioactive moiety that is a therapeutic agent, wherein the third radioactive moiety is selected from a group consisting of ¹⁶¹Tb, ²²⁵Ac, ¹⁶¹Tb/²²⁵Ac, ¹⁷⁷Lu, ¹³⁴Ce, ¹⁴⁰Nd, ¹⁶⁹Er, ¹³⁴Ce/¹³⁴La, and ¹⁴⁰Nd/¹⁴⁰Pr.

In another aspect, a method of treating cancer in a subject is provided. In some embodiments, the method comprises detecting the presence of a tumor in the subject by administering a first radiolabeled antibody comprising a syndecan 1 (SDC1) antibody or antibody fragment thereof conjugated to a first chelating agent and a first radioactive moiety that is an imaging agent; and treating the subject by administering a therapeutically effective amount of a second radiolabeled antibody comprising the SDC1 antibody or antibody fragment thereof conjugated to a second chelating agent and a second radioactive moiety that is a therapeutic agent. In some embodiments of the methods, the SDC1 antibody comprises a heavy chain variable region comprising a CDRH1 comprising SEQ ID NO: 3: a CDRH2 comprising SEQ ID NO: 4; and a CDRH3 comprising SEQ ID NO: 5; and a light chain variable region comprising a CDRL1 comprising SEQ ID NO: 6: a CDRL2 comprising SEQ ID NO: 7; and a CDRL3 comprising SEQ ID NO: 8. In some embodiments, the first radioactive moiety is selected from a group consisting of ⁸⁹Zr, ¹³¹I, ¹²⁵I, ¹²³I, ¹¹¹I, ^{99m}Tc, ⁹⁰Y, ¹⁸⁶Re, ¹⁸⁸Re, ³²P, ¹⁵³Sm, ⁶⁷Ga, ²⁰¹Tl, ⁷⁷Br, or ¹⁸F. In certain embodiments, the first radioactive moiety is ⁸⁹Zr. In some embodiments, the second radioactive moiety is selected from a group consisting of ¹⁶¹Tb, ²²⁵Ac, ¹⁶¹Tb/²²⁵Ac, ¹⁷⁷Lu, ¹³⁴Ce, ¹⁴⁰Nd, ¹⁶⁹Er, ¹³⁴Ce/¹³⁴La, and ¹⁴⁰Nd/¹⁴⁰Pr. In certain embodiments, the second radioactive moiety is ¹⁶¹Tb. In some embodiments, the SDC1 antibody or antibody fragment comprises a heavy chain variable region (VH) having at least 90% identity to SEQ ID NO: 1; and a light chain variable region (VL) having at least 90% identity to SEQ ID NO: 2. In some embodiments, the SDC1 antibody or antibody fragment comprises a heavy chain variable region (VH) comprising SEQ ID NO: 1; and a light chain variable region (VL) comprising SEQ ID NO: 2. In some embodiments, the chelating agent comprises deferoxamine (DFO), 1,4,7,10-tetraazacyclododecane (DO3A), or diethylenetriaminepentaacetic acid (DTPA). In some embodiments, the first radiolabeled antibody comprises the SDC1 antibody or antibody fragment thereof conjugated to DFO and 89Zr, and wherein the second radiolabeled antibody comprises the SDC1 antibody or antibody fragment thereof conjugated to DO3A and ¹⁶¹Tb. In some embodiments, the second radiolabeled antibody is administered at a concentration of about 100 μCi to 300 μCi. In some embodiments, the cancer is a pancreatic cancer, a colorectal cancer, or a non-small cell lung cancer. In some embodiments, the subject has relapsed.

In some embodiments, the methods of treatment further comprise administering at least a second anti-cancer therapy. In some embodiments, the second anti-cancer therapy is a chemotherapy, molecular targeted therapy, immunotherapy, radiotherapy, radioimmunotherapy, phototherapy, gene therapy, surgery, hormonal therapy, epigenetic modulation, anti-angiogenic therapy or cytokine therapy. In some embodiments, the methods comprise administering at least a third radiolabeled antibody comprising the SDC1 antibody or antibody fragment thereof conjugated to a third chelating agent and a third radioactive moiety that is a therapeutic agent, wherein the third radioactive moiety is selected from a group consisting of ¹⁶¹Tb, ²²⁵Ac, ¹⁶¹Tb/²²⁵Ac, ¹⁷⁷Lu, ¹³⁴Ce, ¹⁴⁰Nd, ¹⁶⁹Er, ¹³⁴Ce/¹³⁴La, and ¹⁴⁰Nd/¹⁴⁰Pr.

In another aspect, methods of monitoring a response of a subject with an SDC1-expressing cancer to cancer therapy are provided. In some embodiments, the methods comprise detecting the presence of a tumor in the subject at a first time point by administering a first radiolabeled antibody comprising a syndecan 1 (SDC1) antibody or antibody fragment thereof conjugated to a first chelating agent and a first radioactive moiety that is an imaging agent and obtaining a first image of the tumor; and treating the subject by administering a therapeutically effective amount of a second radiolabeled antibody comprising the SDC1 antibody or antibody fragment thereof conjugated to a second chelating agent and a second radioactive moiety that is a therapeutic agent: detecting the presence or absence of the tumor in the subject at a second time point after the subject has been treated with cancer therapy by administering the first radiolabeled antibody and obtaining a second image of the tumor; and comparing the first image to the second image to determine whether a change in tumor size has occurred. In some embodiments, the SDC1 antibody comprises a heavy chain variable region comprising a CDRH1 comprising SEQ ID NO: 3: a CDRH2 comprising SEQ ID NO: 4; and a CDRH3 comprising SEQ ID NO: 5; and a light chain variable region comprising a CDRL1 comprising SEQ ID NO: 6: a CDRL2 comprising SEQ ID NO: 7; and a CDRL3 comprising SEQ ID NO: 8. In some embodiments, the steps of treating, detecting at a later time point, and comparing the images are repeated at a third time point after the subject receives cancer therapy. In some embodiments, the cancer therapy comprises a combination therapy comprises a second anti-cancer therapy. In some embodiments, the second anti-cancer therapy is a chemotherapy, molecular targeted therapy, immunotherapy, radiotherapy, radioimmunotherapy, phototherapy, gene therapy, surgery, hormonal therapy, epigenetic modulation, anti-angiogenic therapy or cytokine therapy. In some embodiments, the first radioactive moiety is selected from a group consisting of ⁸⁹Zr, ¹³¹I, ¹²⁵I, ¹²³I, ¹¹¹I, ^{99m}Tc, ⁹⁰Y ¹⁸⁶Re ¹⁸⁸Re, ³²P, ¹⁵³Sm, ⁶⁷Ga, ²⁰¹Tl, ⁷⁷Br. or ¹⁸F. In certain embodiments of the methods of monitoring, the first radioactive moiety is ⁸⁹Zr. In some embodiments, the second radioactive moiety is selected from a group consisting of ¹⁶¹Tb, ²²⁵Ac, ¹⁶¹Tb/²²⁵Ac, ¹⁷⁷Lu, ¹³⁴Ce, ¹⁴⁰Nd, ¹⁶⁹Er, ¹³⁴Ce/¹³⁴La, and ¹⁴⁰Nd/¹⁴⁰Pr. In certain embodiments, the second radioactive moiety is ¹⁶¹Tb. In some embodiments of the methods of monitoring, the SDC1 antibody or antibody fragment comprises a heavy chain variable region (VH) having at least 90% identity to SEQ ID NO: 1; and a light chain variable region (VL) having at least 90% identity to SEQ ID NO: 2. In some embodiments, the SDC1 antibody or antibody fragment comprises a heavy chain variable region (VH) comprising SEQ ID NO: 1; and a light chain variable region (VL) comprising SEQ ID NO: 2. In some embodiments, the chelating agent comprises deferoxamine (DFO), 1,4,7,10-tetraazacyclododecane (DO3A), or diethylenetriaminepentaacetic acid (DTPA). In some embodiments, the first radiolabeled antibody comprises the SDC1 antibody or antibody fragment thereof conjugated to DFO and ⁸⁹Zr, and wherein the second radiolabeled antibody comprises the SDC1 antibody or antibody fragment thereof conjugated to DO3A and ¹⁶¹Tb.

### BRIEF DESCRIPTION OF THE DRAWINGS

The present application includes the following figures. The figures are intended to illustrate certain embodiments and/or features of the compositions and methods, and to supplement any description(s) of the compositions and methods. The figures do not limit the scope of the compositions and methods, unless the written description expressly indicates that such is the case.

FIGS. 1A-1F show immunohistochemistry images in a primary human PDAC tissue microarray. SDC1 expression was evaluated in Normal (FIG. 1A), TAP (tumor associated pancreatitis) (FIG. 1B), PanIN1 (Pancreatic Intraepithelial Neoplasia 1) (FIG. 1C), PanIN2 (Pancreatic Intraepithelial Neoplasia 2) (FIG. 1D), PanIN3 (Pancreatic Intraepithelial Neoplasia 3) (FIG. 1E), and PDA (pancreatic ductal adenocarcinoma) (FIG. 1F) tissue.

FIGS. 2A-2B show results of fluorescence activated cell sorting (FACS) analysis of surface Syndecan-1 (SDC1) in iKras (inducible Kras) tumor cells upon doxycycline withdrawal (OFF) and reintroduction (Re-ON).

FIG. 3A is an example of a survival curve of genetically engineered mouse model of pancreatic cancer driven by pancreatic-specific expression of Kras^{G12D} (Kras) and heterozygous deletion of p53 (p53 L/+) with SDC1 wt or −/−allele, and FIG. 3B shows microscopy images of inducible knockdown of SDC1 suppressed tumor growth in an orthotopic xenograft model derived from PDX PATC53 cells.

FIG. 4A provides examples of microscopy images of a macropinocytosis assay in AsPC1, PATC69 PDAC cells expressing mouse SDC1, or PATC69 cells transfected with the empty vector (without SDC1) that were infected with scrambled shRNA (SCR), shSDC1-1 or shSDC1-2. The quantified results for macropinocytosis index are shown in FIG. 4B. **P<0.01: ***P<0.001; and NS indicates no significance.

FIG. 5A provides images of immunohistochemistry and H&E staining (hematoxylin and eosin) for SDC1 and p-ERK in tumors tissues of iKras tumors and iKras-relapse tumor from an iKras model. FIG. 5B provides results of a FACS analysis of surface SDC1 in iKras tumor cells and iKras-escaper cells upon doxycycline withdrawal and iKras-escaper lines.

FIG. 6 shows data from a clonogenic assay of iKras cells (bottom panel) and iKras-escaper cells (top panel) upon a shRNA-mediated SDC1 knockdown.

FIG. 7A is a graph showing surface SDC1 expression (norm. mean fluorescence intensity (MFI)) measured by FACS analysis upon doxycycline withdrawal in iKras cells after the number of days indicated on the x axis (FIG. 7A). FIG. 7B is a graph showing surface SDC1 expression (norm. MFI) measured by FACS analysis upon AMG510 treatment (FIG. 7B) in PDAC cell line MIA PaCa2 (left bar in each pair) and colorectal cancer (CRC) cell line SW837 (right bar in each pair) after the number of days indicated on the x axis. **P<0.01; ***P<0.001.

FIG. 8A shows images from a clonogenic assay of parental MIA PaCa2 or SW837 cells and their derived AMG510-resistant (AMG510-R) cells upon shRNA-mediated knockdown of SDC1 in the presence of AMG510 or scrambled RNA (control). FIG. 8B shows images of tumors in mice from an assay in which SDC1 expression bypassed Kras^{G12D} extinction-induced tumor regression upon doxycycline withdrawal in orthotopic xenografts generated from iKras cells (bottom panel). GFP-expressing cells were used as a negative control (top panel), and cells ectopically expressing KRAS^{G12D} were used as a positive control (middle panel).

FIG. 9A is a graph showing data from a cell viability assay of MIA PaCa2 cells harboring shScr or shSDC1 and treated with AMG510. FIG. 9B is a graph showing SubQ tumor growth of parental MIA PaCa2 (MIA) or AMG510-resistant (AMG510R) cells harboring shScr or shSDC1 and treated with AMG510 (A) or vehicle (V). Treatment started when tumors reached about 150 mm³. *P<0.05; **P<0.01; and ***P<0.001.

FIG. 10A shows examples of SQ tumor growth curves of CRC PDXs that are sensitive (left panel), acquiring resistance (middle panel), or intrinsically resistant to AMG510 treatment (right panel), and FIG. 10B shows images from IHC analysis of SDC1 in these models.

FIGS. 11A-11C show the binding characteristics of the 22B SDC1 monoclonal antibody. FIG. 11A is graph showing the binding percent of the clone 22B antibody and commercially available antibodies nBT062 and MI15 to recombinant human SDC1 protein by ELISA. FIG. 11B shows results of an affinity test of 22B by OCTET analysis, and FIG. 11C is a plot of flow cytometry analysis of 22B binding with human PDAC PATC53 cells with endogenous SDC1 or CRISPR-mediated SDC1 deletion.

FIG. 12 is a graph showing the reactivity of SDC1 monoclonal antibodies (22B and commercial antibodies nBT062 and MI15) or control antibody (mIgG2a) to recombinant cynomolgus SDC1 protein.

FIG. 13 is a graph of the quantification of macropinocytosis index in PATC53 cells treated with PBS, mIgG2a, 22B, or nBT062 antibodies. ***P<0.001; and NS is non-significance.

FIG. 14A is a graph showing the results of the evaluation of ADCC effect of mIgG2a isotype control or defucosylated 22B by FACS using PATC53 as target cells (T) and isolated human PBMC as effector cells (E). FIGS. 14B and 14C are graphs showing results of ADCC reporter assays using human PATC53 cells and lymphocyte U266 cells (FIGS. 14B and 14C, respectively.

FIGS. 15A-15B are graphs of an SQ xenograft model of AsPC1 in nude mice treated with mIgG2a isotype control, wildtype 22B, or nBT062 (FIG. 15A), or with mIgG2a isotype control or defucosylated 22B (FIG. 15B). FIG. 15C is a graph showing results of an SQ xenograft model of Panc02-hSDC1 in C57BL/6NJ mice treated with mIgG2a isotype or defucosylated 22B.

FIGS. 16A-16D are graphs of FACS analysis of tumor models (from FIG. 15). Tumors were subjected to staining for certain markers and FACS analysis. Results are shown as the percentage of cells for staining for CD45 (FIG. 16A), CD69 (FIG. 16B), PD1 (FIG. 16C) or PDL1 (FIG. 16D). *P<0.05: **P<0.01: ***P<0.001; and ****P<0.0001.

FIG. 17 is a graph of the tumor volume over time in SQ xenograft models of Panc02-hSDC1 in C57B/NJ mice treated with mIgG2a isotype (CTR), aPD1, Def-22B+aPD1, or Def-22b antibody.

FIGS. 18A-18B are graphs showing the internalization of the 22B antibody by PATC53 or AsPc1 cells as examined by InCucyte. Serial diluted 22B antibody (circles), mouse IgG2a isotype control (squares), or no antibodies (triangles) were mixed with FabFluor-pH dye and incubated with PATC53 or AsPc1 cells for 0-96 hours as indicated on the x axis, and the fluorescence was detected. Antibodies that were internalized and entered into a lysosome showed red fluorescence (shown as RCU on the y axis). FIGS. 18A-18B show the results, with error bars, where 4 μg/mL of antibody was used.

FIGS. 19A-19D provide data showing the suppression of tumor progression by the 22B antibody in PDAC models. FIGS. 19A-19C are graphs showing tumor volume in AsPc1 cells (FIG. 19A). PATC53 cells (FIG. 19B), or Panc02-hSDC1 cells (FIG. 19C) after treatment with the 22B antibody (circles) or an IgG2a control (squares) when tumors reached 50-100 mm³. FIG. 19D shows exemplary MRI images of orthographic xenograft tumors after treatment with 22B (top panel) or the IgG2a control antibody (bottom panel).

FIGS. 20A-20C are graphs demonstrating the effect of combination therapies on the tumor-suppressive activity of the 22B antibody. FIG. 20A shows the volume of subcutaneous xenograft tumors derived from mouse PDAC cell line Pan02 expressing human SDC1 after treatment with the 22B antibody (circles), a PD1 antibody (triangles), a combination of 22B and PD1 antibodies (squares), or a control antibody (inverted triangles). FIG. 20B shows the volume of subcutaneous xenograft tumors derived from mouse PDAC cell line Pan02 expressing human SDC1 after treatment with the 22B antibody (circles), a 4-1BB antibody (triangles), a combination of 22B and 4-1BB antibodies (squares), or a control antibody (inverted triangles). FIG. 20C shows the volume of subcutaneous xenograft tumors derived from human patient derived PDAC cells PATC153 after treatment with the 22B antibody (circles), gemcitabine (triangles), a combination of 22B and gemcitabine (squares), or a control antibody (inverted triangles).

FIGS. 21A-21C provide data demonstrating the effect of combination therapies on the tumor-suppressive activity of the 22B antibody. FIG. 21A provides FACS data for MiaPaca1 PDAC cells or PATC53 PDAC cells after treatment with the Kras inhibitor AMG510 (left panel) or MRTX1133 (right panel). FIG. 21B is a graph showing the volume of subcutaneous xenograft tumors derived from mouse PDAC cell line HY50760 expressing human SDC1 after treatment with the 22B antibody (circles), AMG510 (triangles), a combination of 22B and AMG510 (squares), or a control antibody (inverted triangles). FIG. 21C is a graph showing the volume of subcutaneous xenograft tumors derived from PDAC cell line AsPc1 after treatment with the 22B antibody (circles), MRTX1133 (triangles), a combination of 22B and MRTX1133 (squares), or a control antibody (inverted triangles).

FIG. 22 is an amino acid sequence alignment of human SDC1 (SEQ ID NO: 23) and mouse SDC1 (SEQ ID NO: 24). The amino acids required for binding of the 22B antibody and the nBT062 antibody are indicated with a box around the amino acids.

FIG. 23 is a graph showing the in vitro cellular uptake of 89Zr-labeled antibodies ([89Zr]Zr-DFO-22B) at 2 hours in PDAC cell lines. ***P<0.001. ****P<0.0001 one-way ANOVA followed by Dunnett's multiple comparison test.

FIG. 24 provides in vivo positron emission tomography (PET) images of a xenograft tumor-bearing mouse model, using [89Zr]Zr-DFO-22B in AsPC-1 PDAC cells (syndecan-1, (SDC1+)) (left image), [89Zr]Zr-DFO-22B in SDC1 knockout cells (middle panel), or a control [89Zr]Zr-DFO-IgG in AsPC-1 PDAC cells (right image).

FIGS. 25A-25E provide in vivo micro positron emission tomography-computed tomography (microPET/CT) images of a mouse bearing an SDC1+PATC53 cell tumor that was administered [89Zr]Zr-DFO-22B (FIG. 25A), a mouse bearing a PATC53 cell tumor that was administered 89Zr-labeled non-targeting control IgG ([⁸⁹Zr]Zr-DFO-IgG) (FIG. 25B), a mouse bearing an AsPC-1 SDC1 knockout tumor that was administered [89Zr]Zr-DFO-22B (FIG. 25C), and a mouse bearing a PATC53 cell tumor that was administered both [89Zr]Zr-DFO-22B and an excess amount of nonradioactive 22B (FIG. 25D), at 24, 48, and 120 hour post-injection. The circle indicates the location of the tumor. FIG. 25E shows autoradiography images of the representative tumor from the xenografts of FIGS. 25A-25D.

FIG. 26A is a graph showing the biodistribution of 89Zr-labeled antibodies in selected organs isolated from AsPC-1 xenograft-bearing mice. FIG. 26B is a graph showing the tumor-to pancreas ratio of ⁸⁹Zr-labeled antibodies measured post injection. ****P<0.0001, one-way ANOVA followed by Dunnett's multiple comparison test.

FIG. 27 is a graph showing the biodistribution of ¹⁶¹Tb-labeled 22B antibodies in selected organs isolated from AsPC-1 xenograft-bearing mice at 120 hour post-injection of 50 μg of 22B (left bar for each organ), 30 μg of 22B (middle bar for each organ), or 3 μg of 22B (right bar for each organ).

FIG. 28 is a graph showing the body weight change of mice administered with [¹⁶¹Tb]Tb-DO3A-22B at incremented radioactivity doses (150 μCi (diamonds), 200 μCi (inverted triangles), 250 μCi (triangles), and 300 μCi (squares)). The antibody was administered through the tail vein, and changes in body weight and other signs of distress were monitored for 21 days.

FIGS. 29A-29D are spider plots depicting the tumor growth of individual mouse models after the treatment. FIG. 29A shows the tumor growth in a mouse model after no treatment (control). FIG. 29B shows the tumor growth in a mouse after treatment with 100 μCi of [¹⁶¹Tb]Tb-DO3A-isotype control IgG. FIG. 29C shows the tumor growth in a mouse after treatment with 100 μCi of [161Tb]Tb-DO3A-22B. FIG. 29D shows the tumor growth in a mouse after treatment with 200 μCi of [161Tb]Tb-DO3A-22B.

FIG. 30A is a graph of the tumor doubling time (days) for AsPC-1 SDC1+ xenografts. The tumor doubling time for [¹⁶¹Tb]Tb-DO3A-22B treated groups (26±8.99 days for 100 μCi and 31.8±5.89 days for 200 μCi) was significantly greater than the non-treated group (10.1±5.79 days). FIG. 30B is a graph of the body weight (grams) of mice administered with PBS, 100 μCi of [¹⁶¹Tb]Tb-DO3A-isotype control IgG, 100 μCi of [¹⁶¹Tb]Tb-DO3A-22B, or 200 μCi of [¹⁶¹Tb]Tb-DO3A-22B, showing no significant acute toxicity post treatment for 50 days. FIG. 30C provides a graph and table showing the results of a log-rank test demonstrating the survival benefit of [¹⁶¹Tb]Tb-DO3A-22B treatment.**P<0.01, ***P<0.001, one-way ANOVA followed by Dunnett's multiple comparison test.

### DETAILED DESCRIPTION

The following description recites various aspects and embodiments of the present compositions and methods. No particular embodiment is intended to define the scope of the compositions and methods. Rather, the embodiments merely provide non-limiting examples of various compositions and methods that are at least included within the scope of the disclosed compositions and methods. The description is to be read from the perspective of one of ordinary skill in the art: therefore, information well known to the skilled artisan is not necessarily included.

### I. Introduction

Due to its high prevalence in cancer types and central role in tumor progression, the effective and specific targeting of mKRAS has been a continuing priority in anti-cancer drug development. Effective therapeutic options for patients with PDAC remain a critical challenge. Cell surface localization of Syndecan-1 (SDC1, also referred to as CD138) is essential for disease maintenance and progression, where it regulates macropinocytosis, an essential metabolic pathway that fuels PDAC cell growth. SDC1 was selected as an attractive target suitable for PDAC, including for a theranostics approach, as its upregulated expression and rapidly internalizing feature enable cancer cell-selective targeting of radionuclidic payloads. Moreover, previous studies have revealed SDC1 as a surrogate marker of mutant KRAS and delineated its pivotal role in promoting tumor malignancy, underscoring the therapeutic promise of targeting SDC1 in PDAC.

The present disclosure provides therapeutic compositions that can be used to treat patients in which mKRAS has been activated. In particular, the disclosure provides antibodies and fragments thereof that specifically bind syndecan 1 (SDC1, also referred to as CD138). Also provided are chimeric antigen receptors that specifically bind SDC1. The present disclosure also provides a theranostic pair of radionuclide antibody conjugates that specifically bind syndecan 1 (SDC1, also referred to as CD138), as well as methods of using the theranostic pair for cancer treatment and diagnostics.

### II. Definitions

Unless otherwise defined, all terms of art, notations, and other scientific or medical terms or terminology used herein are intended to have the meanings commonly understood by those of ordinary skill in the art. In some cases, terms with commonly understood meanings are defined herein for clarity and/or for ready reference, and the inclusion of such definitions herein should not be construed as representing a substantial difference over the definition of the term as generally understood in the art.

Articles “a” and “an” are used herein to refer to one or to more than one (i.e., at least one) of the grammatical object of the article. By way of example, “an element” means at least one element and can include more than one element.

The use herein of the terms “including.” “comprising.” or “having.” and variations thereof, is meant to encompass the elements listed thereafter and equivalents thereof as well as additional elements. Embodiments recited as “including.” “comprising,” or “having” certain elements are also contemplated as “consisting essentially of and “consisting of those certain elements. As used herein, “and/or” refers to and encompasses any and all possible combinations of one or more of the associated listed items, as well as the lack of combinations where interpreted in the alternative (“or”).

As used herein, the transitional phrase “consisting essentially of” (and grammatical variants) is to be interpreted as encompassing the recited materials or steps “and those that do not materially affect the basic and novel characteristic(s)” of the claimed invention. See, e.g., In re Herz, 537 F.2d 549, 551-52 (CCPA 1976) (emphasis in the original); see also MPEP § 2111.03. Thus, the term “consisting essentially of” as used herein should not be interpreted as equivalent to “comprising.”

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure.

The terms “about” and “approximately” as used herein shall generally mean an acceptable degree of error for the quantity measured given the nature or precision of the measurements. Exemplary degrees of error are within 20% (%); preferably, within 10%; and more preferably, within 5% of a given value or range of values. Any reference to “about X” or “approximately X” specifically indicates at least the values X, 0.95X, 0.96X, 0.97X, 0.98X, 0.99X, 1.01X, 1.02X, 1.03X, 1.04X, and 1.05X. Thus, expressions “about X” or “approximately X” are intended to teach and provide written support for a claim limitation of, for example, “0.98X.” Numerical quantities given herein are approximate unless stated otherwise, meaning that the term “about” or “approximately” can be inferred when not expressly stated. When “about” is applied to the beginning of a numerical range, it applies to both ends of the range.

As used throughout, the terms “nucleic acid,” “nucleic acid sequence,” “oligonucleotide,” “nucleotides,” or other grammatical equivalents as used herein mean at least two nucleotides, either deoxyribonucleotides or ribonucleotides, or analogs thereof, covalently linked together. Polynucleotides are polymers of any length, including. e.g., 20, 50, 100, 200, 300, 500, 1000, 2000, 3000, 5000, 7000, 10,000, etc. A polynucleotide described herein generally contains phosphodiester bonds, although in some cases, nucleic acid analogs are included that may have at least one different linkage, e.g., phosphoramidate, phosphorothioate, phosphorodithioate, or O-methylphophoroamidite linkages, and peptide nucleic acid backbones and linkages. Mixtures of naturally occurring polynucleotides and analogs can be made; alternatively, mixtures of different polynucleotide analogs, and mixtures of naturally occurring polynucleotides and analogs may be made. The following are non-limiting examples of polynucleotides: a gene or gene fragment, exons, introns, messenger RNA (mRNA), transfer RNA, ribosomal RNA, ribozymes, cDNA, CRNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component. The term also includes both double- and single-stranded molecules. Unless otherwise specified or required, the term polynucleotide encompasses both the double-stranded form and each of two complementary single-stranded forms known or predicted to make up the double-stranded form. A polynucleotide is composed of a specific sequence of four nucleotide bases: adenine (A), cytosine (C), guanine (G), thymine (T), and uracil (U) for thymine when the polynucleotide is RNA. Thus, the term “polynucleotide sequence” is the alphabetical representation of a polynucleotide molecule. Unless otherwise indicated, a particular polynucleotide sequence also implicitly encompasses conservatively modified variants thereof (e.g., degenerate codon substitutions) and complementary sequences as well as the sequence explicitly indicated. Specifically, degenerate codon substitutions may be achieved by generating sequences in which the third position of one or more selected (or all) codons is substituted with mixed-base and/or deoxyinosine residues.

Unless otherwise indicated, a particular nucleic acid sequence also implicitly encompasses conservatively modified variants thereof, alleles, orthologs, SNPs, and complementary sequences as well as the sequence explicitly indicated.

The terms “polypeptide.” “protein,” and “peptide” are used interchangeably herein to refer to a polymer of amino acid residues in a single chain. The terms apply to amino acid polymers in which one or more amino acid residue is an artificial chemical mimetic of a corresponding naturally occurring amino acid, as well as to naturally occurring amino acid polymers and non-naturally occurring amino acid polymers. Amino acid polymers may comprise entirely L-amino acids, entirely D-amino acids, or a mixture of L and D amino acids. The term “protein” as used herein refers to either a polypeptide or a dimer (i.e., two) or multimer (i.e., three or more) of single chain polypeptides. The single chain polypeptides of a protein may be joined by a covalent bond, e.g., a disulfide bond, or non-covalent interactions. The terms “portion” and “fragment” are used interchangeably herein to refer to parts of a polypeptide, nucleic acid, or other molecular construct.

The amino acids in the polypeptides described herein can be any of the 20 naturally occurring amino acids, D-stereoisomers of the naturally occurring amino acids, unnatural amino acids and chemically modified amino acids. Unnatural amino acids (that is, those that are not naturally found in proteins) are also known in the art, as set forth in, for example, Zhang et al., 2013, “Protein engineering with unnatural amino acids,” Curr. Opin. Struct. Biol. 23 (4): 581-87; Xie et al., 2005, “Adding amino acids to the genetic repertoire,” Curr. Opin. Chem. Biol. 9 (6): 548-54; and all references cited therein. Beta and gamma amino acids are known in the art and are also contemplated herein as unnatural amino acids.

As used herein, a chemically modified amino acid refers to an amino acid whose side chain has been chemically modified. For example, a side chain can be modified to comprise a signaling moiety, such as a fluorophore or a radiolabel. A side chain can also be modified to comprise a new functional group, such as a thiol, carboxylic acid, or amino group. Post-translationally modified amino acids are also included in the definition of chemically modified amino acids.

The term “identity” or “substantial identity,” as used in the context of a polynucleotide or polypeptide sequence described herein, refers to a sequence that has at least 60% sequence identity to a reference sequence. Alternatively, percent identity can be any integer from 60% to 100%. Exemplary embodiments include at least: 60%, 65%, 70%, 75%, 80%, 85%, 88%, 90%, 91%, 92%, 93%, 94%, 95%, 96%, 97%, 98%, or 99%, as compared to a reference sequence using the programs described herein; preferably BLAST using standard parameters, as described below. One of ordinary skill in the art will recognize that these values can be appropriately adjusted to determine corresponding identity of proteins encoded by two nucleotide sequences by taking into account codon degeneracy, amino acid similarity, reading frame positioning and the like. In some embodiments, the polynucleotide or polypeptide has at least 60%, 65%, 70%, 75%, 80%, 85%, 88%, 90%, 91%, 92%, 93%, 94%, 95%, 96%, 97%, 98%, or 99% identity with any one of SEQ ID NOS: 1-24.

For sequence comparison, typically one sequence acts as a reference sequence to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window;” as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison may be conducted by the local homology algorithm of Smith & Waterman, 1981, Add. APL. Math. 2:482, by the homology alignment algorithm of Needleman & Wunsch, 1970, J. Mol. Biol. 48:443, by the search for similarity method of Pearson & Lipman, 1988, Proc. Natl. Acad. Sci. (U.S.A.) 85:2444, by computerized implementations of these algorithms (e.g., BLAST), or by manual alignment and visual inspection.

Algorithms that are suitable for determining percent sequence identity and sequence similarity are the BLAST and BLAST 2.0 algorithms, which are described in Altschul et al., 1990, J. Mol. Biol. 215:403-10 and Altschul et al., 1977, Nucleic Acids Res. 25:3389-402, respectively. Software for performing BLAST analyses is publicly available through the National Center for Biotechnology Information (NCBI) web site. The algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al. (1977)). These initial neighborhood word hits acts as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0)) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below; due to the accumulation of one or more negative-scoring residue alignments; or the end of either sequence is reached. The BLAST algorithm parameters W. T, and X determine the sensitivity and speed of the alignment. The BLASTN program (for nucleotide sequences) uses as defaults a word size (W) of 28, an expectation (E) of 10, M=1, N=−2, and a comparison of both strands. For amino acid sequences, the BLASTP program uses as defaults a word size (W) of 3, an expectation (E) of 10, and the BLOSUM62 scoring matrix (see Henikoff & Henikoff, 1989, Proc. Natl. Acad. Sci. USA 89:10915).

The BLAST algorithm also performs a statistical analysis of the similarity between two sequences (see. e.g., Karlin & Altschul, 1993, Proc. Nat'l. Acad. Sci. USA 90:5873-87). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.01, more preferably less than about 10⁻⁵, and most preferably less than about 10⁻²⁰.

The term “theranostic” or “theranostics” as used herein shall generally mean a medical technique that combines diagnosis and treatment to treat cancer or another disease or condition. Theranostics in nuclear medicine refers to the use of radioactive compounds to image biologic phenomena (by detecting expression of specific disease targets such as cell surface receptors), and then to use specifically designed agents to deliver ionizing radiation to the tissues that express these targets. For example, theranostics may refer to a two-pronged approach to diagnosing and treating cancer through the use of radiotracers. As used herein, a “theranostic pair” refers to two agents that bind to a target cell, one agent that is used to image the target cell and one agent that is used to deliver ionizing radiation to the target cell. For example, a theranostic pair may include a first radionuclide conjugated antibody for imaging a target cell to which the antibody binds and a second radionuclide conjugated antibody for delivering ionizing radiation to the target cell.

As used herein, Kras refers to one of the most frequently mutated oncogenes in human cancers. Simanshu et al., 2017, Cell 170:17-33. Mutant KRAS (mKRAS) is involved in a numerous cancers. The present disclosure relates in some embodiments to the effective and specific treatment of mKRAS cancers. Attempts to target mKRAS and other key components of the RAS-MAPK/RAS-PI3K pathway have long produced limited clinical response due to the feedback activation of compensation pathways or high drug toxicity. Notably, recent efforts have resulted in the development of clinically active and highly selective KRAS^{G12C} inhibitors (KRAS^{G12C}i), including MRTX849 (See Hallin, J., et al., 2020, Cancer Discov., 10:54-71) and sotorasib (AMG 510) (Canon J., et al., 2019, Nature, 575:217-23), the latter of which has been approved by the U. S. Food and Drug Administration (FDA) for the treatment of KRAS^{G12C}-mutated NSCLC. However, despite sotorasib achieving the relatively high overall response rate (ORR) of approximately 40% in NSCLC patients, the duration of response remains short-lived, with a median progression-free survival of only 4-6 months (Hong. D. S., et al., 2020, Engl. J. Med., 383:1207-17, Skoulidis, F., et al., 2020, N. Engl. J. Med., 384:2371-81), consistent with the rapid development of acquired resistance. Moreover, in contrast to the therapeutic benefit achieved in NSCLC, KRAS^{G12C} inhibitors are much less effective in CRC with the ORR around just 10% (Fakih et al., 2022, Lancet Oncol., 23:115-24). Agents targeting other KRAS mutants, such as KRAS^{G12D}, are now under preclinical evaluation. For example, MRTX1133 is a small molecule inhibitor that targets KRAS^{G12D}.

Recent studies using preclinical models have indicated that acquired resistance to KRAS^{G12C} inhibition may mediate the activation of multiple receptor tyrosine kinase (RTK) regulators, including EGFR, FGFR, IGFIR, HER2 and SHP2, as well as activation of RAS effectors, such as MYC and mTOR (Hallin et al., 2020; Awad et al., 2021, N. Engl. J. Med., 384:2382-93: Collisson et al., 2011, Nat. Med., 17:500-03 (2011); Misale et al., (2019) Clin. Cancer Res., 25:796-807: Ryan et al., 2020, Clin Cancer Res, 26:1633-43. Using a genetically engineered mouse (GEM) model of PDAC driven by inducible KRAS^{G12D}, it was demonstrated that the activation of the YAP1 oncogene may drive resistance to the genetic inactivation of mKRAS, which has been further confirmed in mKRAS-driven colorectal cancer (CRC) (Tu et al., 2019, JCI Insight; Zhao et al., 2021, Nature 599:679-83; Kapoor et al., 2014, Cell, 158:185-97. Moreover, trophic factors from the tumor microenvironment have also been shown to contribute to the escape from KRAS-addiction in PDAC (Hou et al., 2020, Cancer Discov., 10:1058-77. However, the molecular mechanisms underlying the re-activation of the RTK-RAS signaling pathway as well as the contribution of YAP1 activation to developing resistance to mKRAS inhibitors were not clear.

Multiple strategies have been developed to target SDC1 due to its overexpression on multiple myeloma cells. Most notably, BT062-DM4 and B-B4-1131 are the same SDC1-targeting mAb (clone BT062) but conjugated to the cytotoxic agent DM4 or a radioactive isotope, respectively. These antibodies are being investigated for treatment of multiple myeloma. However, functional antibodies that directly and specifically target the oncogenic function of surface SDC1 have not been developed.

Syndecan 1 (SDC1, also known as CD138), is a cell surface proteoglycan, that has been found to be a key effector downstream of mKRAS, and mKRAS-driven SDC1 membrane expression plays a critical role in PDAC progression and maintenance. Surface SDC1 expression is tightly correlated with acquired resistance to genetic or pharmacological inhibition of mKRAS in both PDAC and CRC preclinical models. Interestingly, the YAP1 oncogene is the major driver for SDC1 reactivation in cells resistant to mKRAS inhibition. YAP1 is a driving force of SDC1 reactivation in tumor cells capable of mKRAS-independent growth and proliferation after chronic inhibition of mKRAS signaling. Early studies elucidated a critical role for the YAP1-SDC1 axis for activating multiple RTKs to establish acquired resistance to mKRAS blockade, thus demonstrating the translational potential of targeting the YAP1-SDC1 axis to overcome the resistance to mKRAS-targeted therapies. (Yao et al., Nature, 2019, 568 (7752): 410-414).

As such, provided herein are antibodies and antigen binding portions thereof that specifically bind to SDC1 (CD138). Also provided herein are various compositions of such antibodies or antigen binding fragments thereof, recombinant nucleic acids encoding the antibodies and antigen binding portions thereof, and associated methods of use. The disclosed monoclonal SDC1 antibody is the first SDC1-specific, de-fucosylated, functional antibody that is capable of directly suppressing SDC1 while maintaining a high binding affinity and high anti-tumor efficacy. In some embodiments, the isolated antibody or antibody fragment specifically binds SEQ ID NO: 22. The antibodies and antigen binding portions thereof, and associated methods provided herein, represent a novel approach for treating and diagnosing patients with mKRAS mutated carcinomas and other cancers that express SDC1. Also provided herein is a theranostic pair for PDAC, prioritizing SDC1, a target that is ubiquitously and highly expressed in PDAC cells. In particular, the theranostic pair specifically binds to SDC1 (CD138).

### III. Antibodies

In one aspect, the present disclosure provides antibodies and antigen binding portions thereof that bind specifically to SDC1. As used herein, the term “antibody” encompasses, but is not limited to, whole immunoglobulin (i.e., an intact antibody) of any class. Native antibodies are usually heterotetrametric glycoproteins, composed of two identical light (L) chains and two identical heavy (H) chains. Typically, each light chain is linked to a heavy chain by one covalent disulfide bond, while the number of disulfide linkages varies between the heavy chains of different immunoglobulin isotypes. Each heavy and light chain also has regularly spaced intrachain disulfide bridges. Each heavy chain has at one end a variable domain (VH or V_{H}) followed by a number of constant domains. Each light chain has a variable domain at one end (VL or V_{L}) and a constant domain at its other end: the constant domain of the light chain is aligned with the first constant domain of the heavy chain, and the light chain variable domain is aligned with the variable domain of the heavy chain. Particular amino acid residues are believed to form an interface between the light and heavy chain variable domains. The light chains of antibodies from any vertebrate species can be assigned to one of two clearly distinct types, called kappa (κ) and lambda (λ), based on the amino acid sequences of their constant domains. Depending on the amino acid sequence of the constant domain of their heavy chains, immunoglobulins can be assigned to different classes. There are five major classes of immunoglobulins: IgA, IgD, IgE, IgG and IgM, and several of these may be further divided into subclasses (isotypes), e.g., IgG-1, IgG-2, IgG-3, and IgG-4; IgA-1 and IgA-2. The heavy chain constant domains that correspond to the different classes of immunoglobulins are called alpha, delta, epsilon, gamma, and mu, respectively. As used herein, the term antibody also encompasses an antibody fragment, for example, an antigen binding fragment. Antigen binding fragments comprise at least one antigen binding domain. One example of an antigen binding domain is an antigen binding domain formed by a VH-VL dimer. Antibodies and antigen binding fragments can be described by the antigen to which they specifically bind. For example, as used herein, the terms “SDC1 antibody” and “anti-SDC1 antibody” both refer to an antibody or fragment thereof that specifically bind SDC1.

The term “variable” is used herein to describe certain portions of the antibody domains that differ in sequence among antibodies and are used in the binding and specificity of each particular antibody for its particular antigen. However, the variability is not usually evenly distributed through the variable domains of antibodies. It is typically concentrated in three segments called complementarity determining regions (CDRs) or hypervariable regions both in the light chain and the heavy chain variable domains. The more highly conserved portions of the variable domains are called the framework (FR). The variable domains of native heavy and light chains each comprise four FR regions, largely adopting a β-sheet configuration, connected by three CDRs, which form loops connecting, and in some cases forming part of, the β-sheet structure. The CDRs in each chain are held together in close proximity by the FR regions and, with the CDRs from the other chain, contribute to the formation of the antigen binding site of antibodies. The constant domains are not involved directly in binding an antibody to an antigen, but exhibit various effector functions, such as participation of the antibody in antibody-dependent cellular toxicity. Each VH and VL generally comprises three CDRs and four FRs, arranged in the following order (from N-terminus to C-terminus): FR1-CDR1-FR2-CDR2-FR3-CDR3-FR4. The CDRs are involved in antigen binding and confer antigen specificity and binding affinity to the antibody. (See Kabat et al. (1991) Sequences of Proteins of Immunological Interest 5th ed., Public Health Service, National Institutes of Health, Bethesda, MD.) CDR sequences on the heavy chain (VH) may be designated as CDRH1, CDRH2, and CDRH3 (alternatively as VHCDR1, VHCDR2, and VHCDR3), while CDR sequences on the light chain (VL) may be designated as CDRL1, CDRL2, and CDRL3 (alternatively as VLCDR1, VLCDR2, and VLCDR3).

The term “epitope,” as used herein, means a component of an antigen capable of specific binding to an antibody or antigen binding fragment thereof. Such components optionally comprise one or more contiguous amino acid residues and/or one or more non-contiguous amino acid residues. Epitopes frequently consist of surface-accessible amino acid residues and/or sugar side chains and can have specific three-dimensional structural characteristics, as well as specific charge characteristics. Conformational and non-conformational epitopes are distinguished in that the binding to the former but not the latter is lost in the presence of denaturing solvents. An epitope can comprise amino acid residues that are directly involved in the binding, and other amino acid residues, which are not directly involved in the binding. The epitope to which an antigen binding protein binds can be determined using known techniques for epitope determination such as, for example, testing for antigen binding protein binding to antigen variants with different point mutations.

As used herein, the terms “binds specifically to,” “specific for,” “binds selectively to” and “selective for” SDC1 or an isoform or an epitope of an SDC1 protein, and the like, mean binding that is measurably different from a non-specific or non-selective interaction. Specific binding can be measured, for example, by determining binding of a molecule compared to binding of a control molecule. Specific binding can also be determined by competition with a control molecule that is similar to the target, such as an excess of non-labeled target. In that case, specific binding is indicated if the binding of the labeled target to a probe is competitively inhibited by the excess non-labeled target.

Provided herein are antibodies and antigen binding portions thereof that bind specifically to SDC1. The SDC1 antibodies and antigen binding portions thereof are polypeptides. As used herein, the terms “antigen binding portion” and “fragment” are used interchangeably to refer to a portion of an antibody polypeptide sequence that binds specifically to SDC1. SDC1-specific antibodies were identified and tested as described in the Examples below. In some embodiments, the antibodies and antigen binding portions thereof provided herein may be a humanized antibody and antigen binding portions thereof.

In one aspect, provided herein is an isolated antibody or antibody fragment, wherein the antibody or antibody fragment comprises: a heavy chain variable region (VH) having at least 90% identity (for example, at least 90%, 91%, 92%, 93%, 94%, 95%, 96%, 97%, 98%, or 99% identical) to SEQ ID NO: 1 and comprising a CDRH1 amino acid sequence comprising SEQ ID NO: 3, a CDRH2 amino acid sequence comprising SEQ ID NO: 4, and a CDRH3 amino acid sequence comprising SEQ ID NO: 5; and a light chain variable region (VL) having at least 90% identity (for example, at least 90%, 91%, 92%, 93%, 94%, 95%, 96%, 97%, 98% or 99% identical) to SEQ ID NO: 2 and comprising a CDRL1 amino acid sequence comprising SEQ ID NO: 6, a CDRL2 amino acid sequence comprising SEQ ID NO: 7, and a CDRL3 amino acid sequence comprising SEQ ID NO: 8.

In another embodiment, the isolated antibody or antibody fragment comprises: a heavy chain variable region (VH) having at least 90% identity (for example, at least 90%, 91%, 92%, 93%, 94%, 95%, 96%, 97%, 98%, or 99% identical) to SEQ ID NO: 11 and a light chain variable region (VL) having at least 90% identity (for example, at least 90%, 91%, 92%, 93%, 94%, 95%, 96%, 97%, 98% or 99% identical) to SEQ ID NO: 12.

In one aspect, provided herein is an isolated antibody or antibody fragment, wherein the antibody or antibody fragment comprises: a heavy chain variable region (VH) having at least 90% identity (for example, at least 90%, 91%, 92%, 93%, 94%, 95%, 96%, 97%, 98%, or 99% identical) to SEQ ID NO: 13 and a light chain variable region (VL) having at least 90% identity (for example, at least 90%, 91%, 92%, 93%, 94%, 95%, 96%, 97%, 98% or 99% identical) to SEQ ID NO: 14.

In some embodiments, heavy chain variable region sequences and light chain variable region sequences encompassed by this disclosure are set forth in Table 1. The CDR sequences in the variable domains listed in Table 1 are indicated by bold and underlined text. In some embodiments, the heavy chain variable region is encoded by a nucleotide sequence having at least 90% identity to SEQ ID NO: 9. In some embodiments, the light chain variable region is encoded by a nucleotide sequence having at least 90% identity to SEQ ID NO: 10.

TABLE 1
Antibody VH and VL amino acid sequences
of selected clone 22B.

| Anti- | | |
|---|---|---|
| body | | |
| ID | VH sequence | VL sequence |
| 22B | SEQ ID NO: 1 | SEQ ID NO: 2 |
| | QVQLQQPGAELAR | MTQTPSSLSASLGDTITITCHAS |
| | PGAAVKLSCKASG | QNINVWLSWYQQKPGNIPKVLIY |
| | YTFTNYWMNWVKQ | KASNLHTGVPSRFSGSGSGTGFT |
| | RPGQGLEWIGMID | LTISSLQPEDIATYYCQQGQSYP |
| | PSDNKTLYNPMFK | LTFGGGTKLEIK |
| | DKATLTVDKSSST | |
| | AYMQLSSLTSEDS | |
| | AVYYCARRGFAYW | |
| | GQGTLVTVSA | |

In some embodiments, the antibody or antigen binding fragment thereof has a heavy chain variable region comprising an amino acid sequence that is at least 90% identical (for example, at least 90%, 91%, 92%, 93%, 94%, 95%, 96%, 97%, 98% or 99% identical) to SEQ ID NO: 1; and a light chain variable region that includes an amino acid sequence that is at least 90% identical (for example, at least 90%, 91%, 92%, 93%, 94%, 95%, 96%, 97%, 98% or 99% identical) to SEQ ID NO: 2.

In some embodiments, the antibody or antigen binding fragment thereof has a heavy chain variable region comprising the CDR1, CDR2, and CDR3 sequences listed in Table 2. In some embodiments, the antibody or antigen binding fragment thereof has a light chain variable region comprising the CDR1, CDR2, and CDR3 sequences listed in Table 2. In some embodiments, the antibody or antigen binding fragment thereof has a light chain variable region comprising the CDR1, CDR2, and CDR3 sequences and a heavy chain variable region comprising the CDR1, CDR2, and CDR3 sequences listed in Table 2.

TABLE 2
CDR amino acid sequences for antibody 22B

| | CDR1 | CDR2 | CDR3 |
|---|---|---|---|
| VH SEQ ID | (SEQ ID | (SEQ ID | (SEQ ID |
| NO: 1 | NO: 3) | NO: 4) | NO: 5) |
| | NYWMN | MIDPSDNK | RGFAY |
| VL SEQ ID | (SEQ ID | (SEQ ID | (SEQ ID |
| NO: 2 | NO: 6) | NO: 7) | NO: 8) |
| | HASQNI | KASN | QQGQS |
| | NVWLS | LHT | YPLT |

The disclosure also provides an antibody or antigen binding portion thereof that specifically binds to SDC1, wherein the antibody or antigen binding portion thereof comprises a heavy chain variable region comprising an amino acid sequence that is at least 90% identical (for example, at least 90%, 91%, 92%, 93%, 94%, 95%, 96%, 97%, 98% or 99% identical) to SEQ ID NO: 1 and a light chain variable region comprising an amino acid sequence that is at least 90% identical (for example, at least 90%, 91%, 92%, 93%, 94%, 95%, 96%, 97%, 98% or 99% identical) to SEQ ID NO: 2. Table 1 provides the sequences for SEQ ID NOs: 1 and 2.

In each case, where a specific amino acid sequence is recited, embodiments comprising a sequence having at least 90% (e.g., 90%, 91%, 92%, 93%, 94%, 95%, 96%, 97%, 98%, or 99%) identity to the recited sequence are also provided.

The amino acid residue sequences provided herein are set forth in single-letter amino acid code which can be used interchangeably with three-letter amino acid code. An amino acid refers to any monomer unit that can be incorporated into a peptide, polypeptide, or protein. The twenty natural or genetically encoded alpha-amino acids are as follows: alanine (Ala or A), arginine (Arg or R), asparagine (Asn or N), aspartic acid (Asp or D), cysteine (Cys or C), glutamine (Gln or Q), glutamic acid (Glu or E), glycine (Gly or G), histidine (His or H), isoleucine (Ile or I), leucine (Leu or L), lysine (Lys or K), methionine (Met or M), phenylalanine (Phe or F), proline (Pro or P), serine (Ser or S), threonine (Thr or T), tryptophan (Trp or W), tyrosine (Tyr or Y), and valine (Val or V). The structures of these twenty natural amino acids are shown in, e.g., Stryer et al., 2002, Biochemistry, 5th ed., Freeman and Company. The term amino acid also includes unnatural amino acids, modified amino acids (e.g., having modified side chains and/or backbones), and amino acid analogs.

The terms identical or percent identity, in the context of two or more nucleic acids or polypeptide sequences, refer to two or more sequences or subsequences that are the same or have a specified percentage of nucleotides or amino acid residues that are the same (e.g., at least 60%, at least 65%, at least 70%, at least 75%, at least 80%, at least 85%, at least 90%, or at least 95% or greater identity over a specified region), when compared and aligned for maximum correspondence over a comparison window, or designated region as measured using one of the following sequence comparison algorithms or by manual alignment and visual inspection.

Identity or similarity with respect to a sequence is defined as the percentage of amino acid residues in the candidate sequence that are identical (i.e., same residue) with the starting amino acid residues, after aligning the sequences and introducing gaps, if necessary, to achieve the maximum percent sequence identity. Methods of alignment of sequences for comparison are well known in the art, e.g., visual alignment or using publicly available software using known algorithms to achieve maximal alignment. Optimal alignment of sequences for comparison can be conducted, for example, by the local homology algorithm of Smith and Waterman (1970, Adv. Appl. Math. 2:482), by the homology alignment algorithm of Needleman and Wunsch (1970, J. Mol. Biol. 48:443), by the search for similarity method of Pearson and Lipman (1988, Proc. Natl. Acad. Sci. USA 85:2444), by computerized implementations of these algorithms (e.g., GAP, BESTFIT, FASTA, and TFASTA in the Wisconsin Genetics Software Package, Genetics Computer Group, 575 Science Dr., Madison, Wis.), or by manual alignment and visual inspection (see, e.g., Ausubel et al., 1995, Current Protocols in Molecular Biology (1995 supplement)). Other publicly available software useful for alignment analysis include BLAST programs, ALIGN, ALIGN-2 (Genentech, South San Francisco, Calif.), and Megalign (DNASTAR).

As with all peptides, polypeptides, and proteins, including fragments thereof, it is understood that additional modifications in the amino acid sequence of the SDC1-specific antibodies or antigen binding fragments thereof described herein, for example, in the heavy chain variable region and/or light chain variable region, can occur that do not alter the nature or function of the antibodies or antigen binding fragments thereof. Such modifications include conservative amino acids substitutions, such that each recited sequence optionally contains one or more conservative amino acid substitutions. The list provided below identifies groups that contain amino acids that are conservative substitutions for one another; these groups are exemplary as other conservative substitutions are known to those of skill in the art:
1) Alanine (A), Glycine (G);
2) Aspartic acid (D), Glutamic acid (E);
3) Asparagine (N), Glutamine (Q);
4) Argininine (R), Lysi ne (K);
5) Isoleucine (I), Leucine (L), Methionine (M), Valine (V);
6) Phenylalanine (F), Tyrosine (Y), Tryptophan (W);
7) Serine(S), Threonine (T); and
8) Cysteine (C), Methionine (M).

By way of example, when an aspartic acid at a specific residue is mentioned, also contemplated is a conservative substitution at the residue, for example, glutamic acid. Non-conservative substitutions, for example, substituting a proline with glycine or substituting a lysine with an asparagine, are also contemplated.

In some instances, the affinity of SDC1-specific antibodies or antigen binding fragments thereof may be optimized through CRISPR to increase or decrease affinity as desired based on one or more of the known characteristics of the binding interaction with SDC1, the structure of either or both of the antibodies or fragments thereof, or the SDC1 protein. For example, in some embodiments, the antibodies or antigen binding fragments disclosed herein, that include defucosylated portions thereof, may have increased binding affinity or specificity when compared to a fucosylated antibody.

Methods of generating and screening for antibodies and antigen binding fragments thereof as provided in this disclosure are described in the Examples and are well-known in the art. Methods of further modifying antibodies for enhanced properties (e.g., enhanced affinity, chimerization, humanization) as well as generating antigen binding fragments, as described herein, are also well-known in the art.

In some embodiments, the heavy chain variable region and/or the light chain variable region of the isolated antibody or antibody fragment has an identical sequence to the heavy chain variable region and/or the light chain variable region of the antibody produced by the methods described herein and, in the Examples, below. In some embodiments, the heavy chain variable region and/or the light chain variable region of the isolated antibody comprises one or more modifications, e.g., amino acid substitutions, deletions, or insertions.

The heavy chain variable region sequence and/or light chain variable region sequence of an antibody described herein can be engineered to comprise one or more variations in the heavy chain variable region sequence and/or light chain variable region sequence. In some embodiments, the engineered variation(s) improves the binding affinity of the antibody for SDC1. In some embodiments, the engineered variation(s) improves the binding affinity of the antibody for SDC1. In some embodiments, the engineered variation(s) decreases the cross-reactivity of the antibody for a second antigen. In some embodiments, the cells used to generate the monoclonal antibody described herein were genetically altered to not express α1,6-fucosyltransferase (α1,6-FucT), wherein the knockdown of (α1,6-FucT) generates a non-fucosylated antibody as described herein.

In some embodiments, the engineered variation is a variation in one or more CDRs, e.g., an amino acid substitution in a heavy chain CDR and/or a light chain CDR as described herein. In some embodiments, the engineered variation is a variation in one or more framework regions, e.g., an amino acid substitution in a heavy chain framework region and/or a light chain framework region. In some embodiments, the engineered variation is a reversion of a region of the heavy chain and/or light chain sequence to the inferred naïve sequence. Methods for determining an inferred naïve immunoglobulin sequence are described in the art. See, e.g., Magnani et al., 2017, PLoS Negl. Trop. Dis., 11: e0005655, doi: 10.1371/journal.pntd.0005655.

In some embodiments, affinity maturation is used to engineer further mutations that enhance the binding affinity of the antibody for SDC1 or enhance the cross-reactivity of the antibody for a second antigen. Methods for performing affinity maturation are known in the art. See, e.g., Renaut et al., 2012, Methods Mol. Biol., 907:451-61.

The present disclosure also encompasses antibodies or fragments thereof that bind to the same epitope of SDC1 as the antibodies disclosed herein. Such antibodies can be identified using routine techniques known in the art, including, for example, competitive binding assays.

The present disclosure also provides chimeric antibodies. The term chimeric antibody refers to an antibody in which a component of the heavy and/or light chain is derived from a particular source or species, while the remainder of the heavy and/or light chain is derived from a different source or species.

A human antibody is one that possesses an amino acid sequence corresponding to that of an antibody produced by a human or a human cell or derived from a non-human source that utilizes a human antibody repertoire or human antibody-encoding sequences (e.g., obtained from human sources, genetically modified non-human sources or designed de novo). Human antibodies specifically exclude humanized antibodies.

Humanized forms of non-human antibodies are chimeric antibodies that contain minimal sequence derived from the non-human antibody. A humanized antibody is generally a human immunoglobulin (recipient antibody) in which residues from one or more CDRs are replaced by residues from one or more CDRs of a non-human antibody (donor antibody). The donor antibody can be any suitable non-human antibody, such as a mouse, rat, rabbit, chicken, or non-human primate antibody having a desired specificity, affinity, or biological effect. In some instances, selected framework region residues of the recipient antibody are replaced by the corresponding framework region residues from the donor antibody. Humanized antibodies can also comprise residues that are not found in either the recipient antibody or the donor antibody. Such modifications can be made to further refine antibody function. (See Jones et al., 1986, Nature, 321:522-25; Riechmann et al., 1988, Nature, 332:323-29; and Presta, 1992, Curr. Op. Struct. Biol., 2:593-96).

In some embodiments, the antibody or antigen binding fragment thereof provided herein can include a heavy (H) chain variable domain sequence (abbreviated herein as VH or V_{H}), and a light (L) chain variable domain sequence (abbreviated herein as VL or V_{L}). In some embodiments, an antibody molecule comprises or consists of a heavy chain and a light chain (sometimes referred to as a half antibody). In another example, and as described further below, an antibody molecule includes two heavy (H) chain variable domain sequences and two light (L) chain variable domain sequence, thereby forming two antigen binding sites, such as Fab, Fab′, F(ab′)₂, Fc, Fd, Fd′, Fv, single chain antibodies (scFv, for example), single variable domain antibodies, diabodies (Dab) (bivalent and bispecific), and chimeric (e.g., humanized) antibodies, which may be produced by the modification of whole antibodies or synthesized de novo using recombinant DNA technologies. These functional antibody fragments retain the ability to bind specifically to their respective antigen. Antibodies and antibody fragments can be from any class of antibodies including, but not limited to, IgG, IgA, IgM, IgD, and IgE, and from any subclass (e.g., IgG1, IgG2, IgG3, and IgG4) of antibodies. The preparation of antibody molecules can be monoclonal or polyclonal. An antibody molecule can also be a human, humanized, CDR-grafted, or an in vitro generated antibody. The antibody can have a heavy chain constant region chosen from, e.g., IgG1, IgG2, IgG3, or IgG4. The antibody can also have a light chain chosen from either kappa or lambda light chains.

As used herein, the term monoclonal antibody refers to an antibody from a population of substantially homogeneous antibodies. A population of substantially homogeneous antibodies comprises antibodies that are the same or substantially similar and that bind the same epitope(s), except for variants that can normally arise during production of the monoclonal antibody. Such variants are generally present in only minor amounts. A monoclonal antibody is typically obtained by a process that includes the selection of a single antibody from a plurality of antibodies. For example, the selection process can be the selection of a unique clone from a plurality of clones, such as a pool of yeast clones, phage clones, bacterial clones, mammalian cell clones, hybridoma clones, or other recombinant DNA clones. The selected antibody can be further altered, for example, to improve affinity for the target, for example, by affinity maturation, to humanize the antibody, to improve its production in cell culture, and/or to reduce its immunogenicity in a subject.

Antigen binding fragments of an antibody molecule are well known in the art, and include, for example, (i) a Fab fragment, a monovalent fragment consisting of the VL, VH, CL and CHI domains; (ii) a F(ab′)₂ fragment, a bivalent fragment comprising two Fab fragments linked by a disulfide bridge at the hinge region; (iii) a Fd fragment consisting of the VH and CHI domains; (iv) a Fv fragment consisting of the VL and VH domains of a single arm of an antibody, (v) a diabody (dAb) fragment, which consists of a VH domain: (vi) a camelid or camelized variable domain: (vii) a single chain Fv (scFv) (See. e.g., Bird et al., 1988 Science 242:423-26: Huston et al., 1988, Proc. Natl. Acad. Sci. USA 85:5879-83); and (viii) a single domain antibody or nanobody. These antibody fragments are obtained using conventional techniques known to those skilled in the art, and the fragments are screened for utility in the same manner as are intact antibodies.

In some embodiments, the monoclonal antibody comprises a heavy chain variable region sequence and a light chain variable region sequence that are derived from an immunoglobulin producing human B cell, and further comprises a kappa or lambda light chain constant region. In some embodiments, the light chain constant region (kappa or lambda) is from the same type of light chain (i.e., kappa or lambda) as the light chain variable region that was derived from the immunoglobulin producing human B cell: as a non-limiting example, if an IgE-producing human B cell comprises a kappa light chain, then the monoclonal antibody that is produced can comprise the light chain variable region from the IgE-producing B cell and further comprises a kappa light chain constant region.

In some embodiments, the monoclonal antibody comprises a heavy chain variable region sequence and a light chain variable region sequence that are derived from an immunoglobulin-producing human B cell, and further comprises a heavy chain constant region having an IgG isotype (e.g., IgG4), an IgA isotype (e.g., IgA1), an IgM isotype, an IgD isotype, or that is derived from an IgG, IgA, IgM, or IgD isotype (e.g., is a modified IgG4 constant region). It will be appreciated by a person of ordinary skill in the art that the different heavy chain isotypes (IgA, IgD, IgE, IgG, and IgM) have different effector functions that are mediated by the heavy chain constant region, and that for certain uses it may be desirable to have an antibody that has the effector function of a particular isotype (e.g., IgG).

In some embodiments, the monoclonal antibody comprises a native (i.e., wild-type) human IgG, IgA, IgM, or IgD constant region. In some embodiments, the monoclonal antibody comprises a native human IgG1 constant region, a native human IgG2 constant region, a native human IgG3 constant region, a native human IgG4 constant region, a native human IgA1 constant region, a native human IgA2 constant region, a native human IgM constant region, or a native human IgD constant region. In some embodiments, the monoclonal antibody comprises a heavy chain constant region that comprises one or more modifications. It will be appreciated by a person of ordinary skill in the art that modifications such as amino acid substitutions can be made at one or more residues within the heavy chain constant region that modulate effector function. In some embodiments, the modification reduces effector function, e.g., results in a reduced ability to induce certain biological functions upon binding to an Fc receptor expressed on an effector cell that mediates the effector function. In some embodiments, the modification (e.g., amino acid substitution) prevents in vivo Fab arm exchange, which can introduce undesirable effects and reduce the therapeutic efficacy of the antibody. See, e.g., Silva et al., 2015, J. Biol. Chem. 280:5462-69.

In some embodiments, the monoclonal antibody comprises a native (i.e., wild-type) human IgM constant region, human IgD constant region, human IgG constant region that is derived from IgG1, IgG2, IgG3, or IgG4, or human IgA constant region that is derived from IgA1 or IgA2 and comprises one or more modifications that modulate effector function. In some embodiments the monoclonal antibody comprises a human IgM constant region, human IgD constant region, human IgG constant region that is derived from IgG1, IgG2, IgG3, or IgG4, or human IgA constant region that is derived from IgA1 or IgA2. In some embodiments, the monoclonal antibody comprises a native (i.e., wild-type) human IgM constant region, human IgD constant region, human IgG constant region that is derived from IgG1, IgG2, IgG3, or IgG4, or human IgA constant region that is derived from IgA1 or IgA2 and comprises one, two, three, four, five, six, seven, eight, nine, ten, or more modifications (e.g., amino acid substitutions). In some embodiments the constant regions includes variations (e.g., one, two, three, four, five, six, seven, eight, nine, ten or more amino acid substitutions) that affect effector function.

In some embodiments the antibody with specified CDRs is an allotype other the allotype(s) found associated with the antibodies produced by the methods described herein and, in the Examples, below: The antibody may comprise an allotype selected from those listed in Table 3 below.

TABLE 3
Human immunoglobulin allotypes.

| | Heavy chains | | | | Light |
|---|---|---|---|---|---|
| Isotype/type | IgG1 | IgG2 | IgG3 | IgA | chains |
| Allotypes | G1m | G2m | G3m | A2m | Km |
| | 1(a) | 23(n) | 21(g1) | 1 | 1 |
| | 2(x) | | 28(g5) | 2 | 2 |
| | 3(f) | | 11(b0) | | 3 |
| | 17(z) | | 5(b1) | | |
| | | | 13 (b3) | | |
| | | | 14 (b4) | | |
| | | | 10 (b5) | | |
| | | | 15(s) | | |
| | | | 16(t) | | |
| | | | 6(c3) | | |
| | | | 24(c5) | | |
| | | | 26(u) | | |
| | | | 27 (v) | | |

NB: Alphabetical notation given within brackets. From: Jefferis and Marie-Paule Lefranc, 2009, “Human immunoglobulin allotypes: Possible implications for immunogenicity” mAbs 1(4): 332-38, incorporated herein by reference.

In some embodiments, a humanized monoclonal antibody comprises CDR sequences, a heavy chain variable region, and/or a light chain variable region as described herein (e.g., as disclosed in Table 1) and further comprises a heavy chain constant region and/or a light chain constant region that is heterologous to the antibody produced by the methods described herein and, in the Examples, below from which the CDR sequences and/or variable region sequences are derived. For example, in some embodiments, the monoclonal antibody comprises the CDR sequences and/or variable region sequences of an antibody produced by the methods described herein and in the Examples below, and further comprises a heavy chain constant region and a light chain constant region that is heterologous to the antibody produced by the methods described herein and in the Examples below (e.g., the heavy chain constant region and/or light chain constant region is a wild-type or modified IgG1, IgG2, IgG3, or IgG4 constant region), or the heavy chain constant region and/or light chain constant region comprises one or more modifications (e.g., amino acid substitutions) relative to the native constant region of the antibodies produced by the methods described herein and in the Examples below.

The antibodies and fragments thereof of this disclosure may comprise variations in heavy chain constant regions to change the properties of the synthetic antibody relative to the corresponding naturally occurring antibody. Exemplary changes include mutations to modulate antibody effector function (e.g., complement-based effector function or FcγR-based effector function), alter half-like, modulate coengagement of antigen and FcγRs, introduce or remove glycosylation motifs (glyco-engineering). See Fonseca et al., 2018, “Boosting half-life and effector functions of therapeutic antibodies by Fc-engineering: An interaction-function review” Int. J. Biol. Macromol. 19:306-11: Wang et al., 2018, “IgG Fc engineering to modulate antibody effector functions” Protein Cell 2018, 9 (1): 63-73: Schlothauer, 2016, “Novel human IgG1 and IgG4 Fc-engineered antibodies with completely abolished immune effector functions,” Protein Engineering. Design and Selection 29 (10): 457-466; Tam et al., 2017, “Functional, Biophysical, and Structural Characterization of Human IgG1 and IgG4 Fc Variants with Ablated Immune Functionality” Antibodies 6, 12, each incorporated herein by reference for all purposes.

Antibody molecules can also be single domain antibodies. Single domain antibodies can include antibodies whose complementary determining regions are part of a single domain polypeptide. Examples include, but are not limited to, heavy chain antibodies, antibodies naturally devoid of light chains, single domain antibodies derived from conventional 4-chain antibodies, engineered antibodies and single domain scaffolds other than those derived from antibodies. Single domain antibodies may be any of the art, or any future single domain antibodies. Single domain antibodies may be derived from any species including, but not limited to mouse, rat, guinea, pig, human, camel, llama, fish, shark, goat, rabbit, and bovine. Single domain antibodies are described, for example, in International Application Publication No. WO 94/04678. For clarity reasons, this variable domain derived from a heavy chain antibody naturally devoid of light chain is known herein as a VHH or nanobody to distinguish it from the conventional VH of four chain immunoglobulins. Such a VHH molecule can be derived from antibodies raised in Camelidae species (e.g., camel, llama, dromedary, alpaca, and guanaco) or other species besides Camelidae.

In some embodiments, an antigen binding fragment can also be or can also comprise, e.g., a non-antibody, scaffold protein. These proteins are generally obtained through combinatorial chemistry-based adaptation of preexisting antigen-binding proteins. For example, the binding site of human transferrin for human transferrin receptor can be diversified using the system described herein to create a diverse library of transferrin variants, some of which have acquired affinity for different antigens. See. e.g., Ali et al., 1999, J. Biol. Chem. 274:24066-73. The portion of human transferrin not involved with binding the receptor remains unchanged and serves as a scaffold, like framework regions of antibodies, to present the variant binding sites. The libraries are then screened, as an antibody library is screened, and in accordance with the methods described herein, against a target antigen of interest to identify those variants having optimal selectivity and affinity for the target antigen. See. e.g., Hey et al., 2005, TRENDS Biotechnol. 23 (10): 514-522.

One of ordinary skill in the art would appreciate that the scaffold portion of the non-antibody scaffold protein can include, e.g., all or part of the Z domain of S. aureus protein A, human transferrin, human tenth fibronectin type III domain, kunitz domain of a human trypsin inhibitor, human CTLA-4, an ankyrin repeat protein, a human lipocalin (e.g., anticalins, such as those described in, e.g., International Application Publication No. WO2015/104406), human crystallin, human ubiquitin, or a trypsin inhibitor from E. elaterium.

Any of the SDC1-specific antibodies or antigen binding fragments thereof described herein can be modified with covalent and/or non-covalent modifications. Such modifications can be introduced into the antibodies or antigen binding fragments by, e.g., reacting targeted amino acid residues of the polypeptide with an organic derivatizing agent that is capable of reacting with selected side chains or terminal residues. Suitable sites for modification can be chosen using any of a variety of criteria including, e.g., structural analysis or amino acid sequence analysis of the antibodies or fragments. Recombinant techniques can be used to modify antibodies or antigen binding fragments thereof. For example, amino acids found to not contribute to either the activity or the binding specificity or affinity of the antibody can be deleted without a loss in the respective activity. Insertions, deletions, substitutions, or other selected modifications of particular regions or specific amino acids residues, provided the activity of the fragment is not significantly altered or impaired compared to the non-modified antibody, or antigen binding fragment thereof can be made. Such methods are readily apparent to a skilled practitioner in the art and can include site specific mutagenesis of the nucleic acid encoding the antibody or fragment thereof. (Zoller et al., 1982, Nucl. Acids Res. 10:6487-500). In some instances, the SDC1-specific antibodies or antigen binding fragments may be labeled by a variety of means for use in diagnostic and/or pharmaceutical applications.

In some embodiments, the antibodies or antigen binding fragments thereof can be conjugated to a heterologous moiety. The heterologous moiety can be, e.g., a heterologous polypeptide, a therapeutic agent (e.g., a toxin or a drug), or a detectable label such as, but not limited to, a radioactive label, an enzymatic label, a fluorescent label, a heavy metal label, a luminescent label, or an affinity tag such as biotin or streptavidin. In some embodiments, the heterologous moiety is an antibody or antigen binding fragment thereof that specifically binds to a different target, and such a conjugated antibody is referred to as a bispecific antibody. For example, in some embodiments, the isolated antibody is a bispecific antibody that specifically binds SDC1 and PD1 or that specifically binds SDC1 and 4-1BB. Additional suitable heterologous polypeptides include, e.g., an antigenic tag (e.g., FLAG (DYKDDDDK) (SEQ ID NO: 15), polyhistidine (6-His: HHHHHH (SEQ ID NO: 16), hemagglutinin (HA; YPYDVPDYA (SEQ ID NO: 17)), glutathione-S-transferase (GST), or maltose-binding protein (MBP)) for use in purifying the antibodies or fragments. Heterologous polypeptides also include polypeptides (e.g., enzymes) that are useful as diagnostic or detectable markers, for example, luciferase, a fluorescent protein (e.g., green fluorescent protein (GFP)), or chloramphenicol acetyl transferase (CAT). Suitable radioactive labels include, e.g., ³²P, ³³P, ¹⁴C, ¹²⁵I, ¹³¹I, ³⁵S, and 3H. In some embodiments, the radioactive label the radioactive moiety is selected from a group consisting of ¹⁶¹Tb, ²²⁵Ac, ¹⁶¹Tb/²²⁵Ac, ⁸⁹Zr, ¹⁷⁷Lu, ¹³⁴Ce, ¹⁴⁰Nd, ¹⁶⁹Er, ¹³⁴Ce/¹³⁴La, and ¹⁴⁰Nd/¹⁴⁰Pr. Suitable fluorescent labels include, without limitation, fluorescein, fluorescein isothiocyanate (FITC), green fluorescent protein (GFP), DyLight™ 488, phycoerythrin (PE), propidium iodide (PI), PerCP, PE-Alexa FluorR 700, Cy5, allophycocyanin, and Cy7. Luminescent labels include, e.g., any of a variety of luminescent lanthanide (e.g., europium or terbium) chelates. For example, suitable europium chelates include the europium chelate of diethylene triamine pentaacetic acid (DTPA) or tetraazacyclododecane-1,4,7,10)-tetraacetic acid (DOTA). Enzymatic labels include, e.g., alkaline phosphatase, CAT, luciferase, and horseradish peroxidase. Another labeling technique which may result in greater sensitivity consists of coupling the antibodies to low molecular weight haptens. These haptens can then be specifically altered by means of a second reaction. For example, it is common to use haptens such as biotin, which reacts with avidin, or dinitrophenol, pyridoxal, or fluorescein, which can react with specific antihapten antibodies. Additional acceptable heterologous moieties are described below in Section VIII.

In some instances, the SDC1 antibody or antigen-binding fragment thereof may be conjugated to an imaging agent. For example, the SDC1 antibody or antigen-binding fragment thereof may be labelled for use in radionuclide imaging. In particular, the agent may be directly or indirectly labelled with a radioisotope. Examples of radioisotopes that may be used are: ²⁷⁷Ac, ²¹¹At, ¹²⁸Ba, ¹³¹Ba, ⁷Be, ²⁰⁴Bi, ²⁰⁵Bi, ²⁰⁶Bi, ⁷⁶Br, ⁷⁷Br, ⁸²Br, ¹⁰⁹Cd, ⁴⁷Ca, ¹¹C, ¹⁴C, ³⁶Cl, ⁴⁸Cr, ⁵¹Cr, ⁶²Cu, ⁶⁴Cu, ⁶⁷Cu, ¹⁶⁵Dy, ¹⁵⁵Eu, ¹⁸F, ¹⁵³Gd, ⁶⁶Ga, ⁶⁷Ga, ⁶⁸Ga, ⁷²Ga, ¹⁹⁸Au, ³H ¹⁶⁶Ho, ¹¹¹In, ^{113m}In, ^{115m}In, ¹²³I, ¹²⁵I, ¹³¹I, ¹⁸⁹Ir, ^{191m} Ir, ¹⁹²Ir, ¹⁹⁴Ir, ⁵²Fe, ⁵⁵Fe, ⁵⁹Fe, ¹⁷⁷Lu, ¹⁵O, ^{191m-191}Os, ¹⁰⁹Pd, ³²P, ³³p, ⁴²K, ²²⁶Ra, ¹⁸⁶Re, ¹⁸⁸Re, ^{82m}Rb, ¹⁵³Sm, ⁴⁶Sc, ⁴⁷Sc, ⁷²Se, ⁷⁵Se, ¹⁰⁵Ag, ²²Na, ²⁴Na, ⁸⁹Sr, ³⁵S, ³⁸S, ¹⁷⁷Ta, ⁹⁶Tc, ^{99m}Tc, ²⁰¹Tl, ²⁰²Tl, ¹¹³Sn, ^{117m}Sn, ¹²¹Sn, ¹⁶⁶Yb, ¹⁶⁹Yb, ¹⁷⁵Yb, ⁸⁸Y, ⁹⁰Y, ⁶²Zn and ⁶⁵Zn. In some embodiments, the radioisotope is ¹³¹I, ¹²⁵I, ¹²³I, ¹¹¹I, ^{99m}Tc, ⁹⁰Y, ¹⁸⁶Re, ¹⁸⁸Re, ³²P, ¹⁵³Sm, ⁶⁷Ga, ²⁰¹Tl, ⁷⁷Br, or ¹⁸F, and is imaged with a photoscanning device. In some embodiments, the radioactive moiety is selected from a group consisting of ¹⁶¹Tb, ²²⁵Ac, ¹⁶¹Tb/²²⁵Ac, ⁸⁹Zr, ¹⁷⁷Lu, ¹³⁴Ce, ¹⁴⁰Nd, ¹⁶⁹Er, ¹³⁴Ce/¹³⁴La, and ¹⁴⁰Nd/¹⁴⁰Pr. Procedures for labeling biological agents with the radioactive isotopes are generally known in the art.

Two proteins (e.g., an antibody and a heterologous moiety) can be cross-linked using any of a number of known chemical cross linkers. Examples of such cross linkers are those that link two amino acid residues via a linkage that includes a “hindered” disulfide bond. In these linkages, a disulfide bond within the cross-linking unit is protected (by hindering groups on either side of the disulfide bond) from reduction by the action, for example, of reduced glutathione or the enzyme disulfide reductase. One suitable reagent, 4-succinimidyloxycarbonyl-α-methyl-α(2-pyridyldithio) toluene (SMPT), forms such a linkage between two proteins utilizing a terminal lysine on one of the proteins and a terminal cysteine on the other. Heterobifunctional reagents that cross-link by a different coupling moiety on each protein can also be used. Other useful cross-linkers include, without limitation, reagents which link two amino groups (e.g., N-5-azido-2-nitrobenzoyloxysuccinimide), two sulfhydryl groups (e.g., 1,4-bis-maleimidobutane), an amino group and a sulfhydryl group (e.g., m-maleimidobenzoyl-N-hydroxysuccinimide ester), an amino group and a carboxyl group (e.g., 4-[p-azidosalicylamido]butylamine), and an amino group and a guanidinium group that is present in the side chain of arginine (e.g., p-azidophenyl glyoxal monohydrate).

Techniques for conjugating a therapeutic moiety (e.g., any of those discussed in Section VIII) to an SDC1-specific antibody or antigen binding fragment thereof as described herein are well known, see, for example, Arnon et al., 1985, Monoclonal Antibodies And Cancer Therapy, Reisfeld et al. (eds.), pp. 243-56; Hellstrom et al., 1987, Controlled Drug Delivery (2nd Ed.), Robinson et al. (eds.), pp. 623-53; Thorpe, 1985, Monoclonal Antibodies '84: Biological And Clinical Applications, Pinchera et al. (eds.), pp. 475-506; “Analysis, Results, And Future Prospective Of The Therapeutic Use Of Radiolabeled Antibody In Cancer Therapy” In: Monoclonal Antibodies For Cancer Detection And Therapy, (Baldwin et al. eds.), pp. 303-316 (1985), and Thorpe et al., 1982, Immunol. Rev. 62:119-158. Alternatively, an antibody can be conjugated to a second antibody to form an antibody heteroconjugate (e.g., a bispecific antibody) as described in U.S. Pat. No. 4,676,980.

In some embodiments, a radioactive label can be directly conjugated to the amino acid backbone of the antibody. Alternatively, the radioactive label can be included as part of a larger molecule (e.g., ¹²⁵I in meta-[¹²⁵I]iodophenyl-N-hydroxysuccinimide ([¹²⁵]mIPNHS), which binds to free amino groups to form meta-iodophenyl (mIP) derivatives of relevant proteins (see. e.g., Rogers et al., 1997, J. Nucl. Med. 38:1221-29) or chelate (e.g., to DOTA or DTPA), which is in turn bound to the protein backbone. Methods of conjugating the radioactive labels or larger molecules/chelates containing them to the antibodies or antigen binding fragments described herein are known in the art. Such methods involve incubating the proteins with the radioactive label under conditions (e.g., pH, salt concentration, and/or temperature) that facilitate binding of the radioactive label or chelate to the protein (see. e.g., U.S. Pat. No. 6,001,329).

Methods for conjugating a fluorescent label (sometimes referred to as a fluorophore) to a protein (e.g., an antibody) are known in the art of protein chemistry. For example, fluorophores can be conjugated to free amino groups (e.g., of lysines) or sulfhydryl groups (e.g., cysteines) of proteins using succinimidyl (NHS) ester or tetrafluorophenyl (TFP) ester moieties attached to the fluorophores. In some embodiments, the fluorophores can be conjugated to a heterobifunctional cross-linker moiety such as sulfo-SMCC. Suitable conjugation methods involve incubating an antibody protein or fragment thereof with the fluorophore under conditions that facilitate binding of the fluorophore to the protein. See. e.g., Welch and Redvanly, (2003), Handbook of Radiopharmaceuticals: Radiochemistry and Applications, John Wiley and Sons.

In some embodiments, the antibodies or fragments can be modified, e.g., with a moiety that improves the stabilization and/or retention of the antibodies in circulation, e.g., in blood, serum, or other tissues. For example, the antibody or fragment can be PEGylated as described in, e.g., Lee et al., 1999, Bioconjug. Chem. 10 (6): 973-78: Kinstler et al., 2002, Advanced Drug Deliveries Reviews 54:477-485; and Roberts et al., 2002, Advanced Drug Delivery Reviews 54:459-476, or HESylated (Fresenius Kabi, Germany) (see. e.g., Pavisić et al., 2010, Int. J. Pharm. 387 (1-2): 110-119). The stabilization moiety can improve the stability, or retention of, the antibody (or fragment) by at least 1.5 (e.g., at least 2, 5, 10, 15, 20, 25, 30, 40, or 50 or more) fold.

In some embodiments, the antibodies or antigen-binding fragments thereof described herein can be glycosylated. In some embodiments, an antibody or antigen-binding fragment thereof described herein can be subjected to enzymatic or chemical treatment, or produced from a cell, such that the antibody or fragment has reduced or absent glycosylation. Methods for producing antibodies with reduced glycosylation are known in the art and described in, e.g., U.S. Pat. No. 6,933,368; Wright et al., 1991, EMBO J. 10 (10): 2717-2723; and Co et al., (1993), Mol. Immunol. 30:1361.

### IV. Chimeric Antigen Receptors

Also provided herein are chimeric antigen receptors comprising any of the antibodies or antigen-binding fragments described herein. Chimeric antigen receptors (CARs, also known as chimeric T cell receptors) are designed to be expressed in host effector cells, e.g., T cells or NK cells, and to induce an immune response against a specific target antigen and cells expressing that antigen. Adoptive T cell immunotherapy, in which a patient's own T lymphocytes are engineered to express CARs, has shown great promise in treating hematological malignancies. CARs can be engineered and used as described, for example, in Sadelain et al., 2013, Cancer Discov. 3:388-98. A CAR typically comprises an extracellular target-binding module, a transmembrane (TM) domain, and an intracellular signaling domain (ICD). The CAR domains can be joined via flexible hinge and/or spacer regions. The extracellular target-binding module generally comprises an antibody or antigen binding fragment thereof. In some instances, multiple binding specificities can be included in the extracellular target-binding module. For example, multiple antibodies or antigen binding fragments thereof that target different antigens can be included to produce bi-specific, tri-specific, or quad-specific CARs. In some embodiments, the CAR antigen binding domain comprises a heavy chain variable region (VH) having a CDRH1 amino acid sequence comprising SEQ ID NO: 3, a CDRH2 amino acid sequence comprising SEQ ID NO: 4, and a CDRH3 amino acid sequence comprising SEQ ID NO: 5; and a light chain variable region (VL) having a CDRL1 amino acid sequence comprising SEQ ID NO: 6, a CDRL2 amino acid sequence comprising SEQ ID NO: 7, and a CDRL3 amino acid sequence comprising SEQ ID NO: 8. TM domains are primarily considered a structural requirement, anchoring the CAR in the cell membrane, and are most commonly derived from molecules regulating T cell function, such as CD8 and CD28. The intracellular module typically consists of the T cell receptor CD35 chain and one or more costimulatory domains from either the Ig (CD28-like) or TNF receptor (TNFR) superfamilies. CARs containing either CD28 or 4-1BB costimulatory domains have been the most widely used, to date, and both of them have yielded dramatic responses in clinical trials. CAR domains are discussed in more detail below.

Provided herein are chimeric antigen receptors comprising: (a) an extracellular target-binding domain comprising an SDC1-specific antibody or antigen binding portion thereof; (b) a transmembrane domain; and (c) a signaling domain.

The extracellular target-binding module of a CAR may comprise an antibody or an antigen-binding fragment thereof that specifically binds a target antigen (e.g., SDC1). In certain embodiments, the extracellular target-binding domain can be a single-chain variable fragment derived from an antibody (scFv), a tandem scFv, a single-domain antibody fragment (V_{H}Hs or sdAbs), a single domain bispecific antibody (BsAbs), an intrabody, a nanobody, an immunokine in a single chain format, Fab, Fab′, or (Fab′)₂ in a single chain format. In other embodiments, the extracellular target-binding domain can be an antibody moiety that comprises covalently bound multiple chains of variable fragments. In some embodiments, the extracellular target-binding domain comprises any of the antibodies or antigen-binding portions thereof described herein. In some embodiments, the extracellular target-binding domain comprises a scFv comprising a heavy chain variable region comprising an amino acid sequence that is at least 90% identical (for example, at least 90%, 91%, 92%, 93%, 94%, 95%, 96%, 97%, 98% or 99% identical) to SEQ ID NO: 1 and a light chain variable region comprising an amino acid sequence that is at least 90% identical (for example, at least 90%, 91%, 92%, 93%, 94%, 95%, 96%, 97%, 98% or 99% identical) to SEQ ID NO: 2. In some embodiments, the extracellular target-binding domain comprises a scFv comprising a heavy chain variable region (VH) having at least 90% identity to SEQ ID NO: 1 and comprising a VHCDR1 amino acid sequence comprising SEQ ID NO3, a VHCDR2 amino acid sequence comprising SEQ ID NO: 4, and a VHCDR3 amino acid sequence comprising SEQ ID NO: 5; and a light chain variable region (VL) having at least 90% identity to SEQ ID NO: 2 and comprising a VLCDR1 amino acid sequence comprising SEQ ID NO: 6, a VLCDR2 amino acid sequence comprising SEQ ID NO: 7, and a VLCDR3 amino acid sequence comprising SEQ ID NO: 8. In some embodiments, the scFv comprises a linker polypeptide between the heavy chain and light chain sequences (e.g., SEQ ID NO: 21 or any of the other linkers described herein).

In some embodiments, the extracellular target-binding domain comprises any of the antibodies or antigen-binding portions thereof described herein. In some embodiments, the extracellular target-binding domain comprises a scFv comprising a heavy chain variable region encoded by a nucleic acid sequence that is at least 90% identical (for example, at least 90%, 91%, 92%, 93%, 94%, 95%, 96%, 97%, 98% or 99% identical) to SEQ ID NO: 9 and a light chain variable region encoded by a nucleic acid sequence that is at least 90% identical (for example, at least 90%, 91%, 92%, 93%, 94%, 95%, 96%, 97%, 98% or 99% identical) to SEQ ID NO: 10.

In some embodiments, the extracellular target-binding domains of the CARs provided herein further comprise one or more additional antigen-binding domains (i.e., in addition to the SDC1-specific antibody or antigen binding portion thereof, as described above). In some embodiments, the extracellular target-binding domain comprises one additional antigen-binding domain. CARs comprising such an extracellular target-binding domain can be referred to as bi-specific CARs. In some embodiments, the extracellular target-binding domain comprises two additional antigen-binding domains. CARs comprising such an extracellular target-binding domain can be referred to as tri-specific CARs. In some embodiments, the extracellular target-binding domain comprises three additional antigen-binding domain. CARs comprising such an extracellular target-binding domain can be referred to as quad-specific CARs. Each of the one or more additional antigen-binding domains may comprise an antibody or antigen binding portion thereof. In some embodiments, the one or more additional antigen-binding domains specifically bind to CD19, CD20, CD22, CD79a, CD79b, or any combination thereof.

The transmembrane domain of a CAR provided herein may be derived from either a natural or from a synthetic source. Where the source is natural, the domain may be derived from any membrane-bound or transmembrane protein. In some embodiments, the transmembrane domain is derived from (i.e., comprises at least the transmembrane region(s) of) the α, β, δ, γ, or ζ chain of the T-cell receptor, CD28, CD3ε, CD3ζ, CD45, CD4, CD5, CD8, CD9, CD16, CD22, CD30, CD33, CD37, CD64, CD80, CD86, CD134, CD137, or CD154. In some embodiments, a transmembrane domain can be chosen based on, for example, the nature of the various other proteins or trans-elements that bind the transmembrane domain or the cytokines induced by the transmembrane domain. In some embodiments, the transmembrane domain comprises a transmembrane domain (e.g., CD8a transmembrane domain). When a transmembrane domain is synthetic, it may comprise predominantly hydrophobic residues such as leucine and valine. In some embodiments, a triplet of phenylalanine, tryptophan, and valine may be found at each end of a synthetic transmembrane domain. In some embodiments, a short oligo- or polypeptide linker, having a length of, for example, between about 2 and about 10) (such as about any of 2, 3, 4, 5, 6, 7, 8, 9, or 10) amino acids in length may form the linkage between the transmembrane domain and the intracellular signaling domain of a CAR described herein. In some embodiments, the linker is a glycine-serine doublet.

The intracellular signaling domain of the CAR is responsible for activation of at least one of the normal effector functions of the immune cell in which the CAR has been placed in or is designed to be placed in. An effector function of a T cell may be, for example, cytolytic activity or helper activity, including the secretion of cytokines. Thus, the term “intracellular signaling domain” refers to the portion of a protein which transduces the effector function signal and directs the cell to perform a specialized function. While usually the entire intracellular signaling domain can be employed, in many cases it is not necessary to use the entire chain. To the extent that a truncated portion of the intracellular signaling domain is used, such truncated portion may be used in place of the intact chain as long as it transduces the effector function signal. The term “intracellular signaling sequence” is thus meant to include any truncated portion of the intracellular signaling domain sufficient to transduce the effector function signal.

Examples of intracellular signaling domains for use in the CARs provided herein include the cytoplasmic sequences of the T cell receptor (TCR) and co-receptors that act in concert to initiate signal transduction following antigen receptor engagement, as well as any derivative or variant of these sequences and any synthetic sequence that has the same functional capability.

It is known that signals generated through the TCR alone are insufficient for full activation of the T cell and that a secondary or costimulatory signal is also required. Thus, T cell activation can be said to be mediated by two distinct classes of intracellular signaling sequence: those that initiate antigen-dependent primary activation through the TCR (primary signaling sequences) and those that act in an antigen-independent manner to provide a secondary or costimulatory signal (costimulatory signaling sequences).

Primary signaling sequences regulate primary activation of the TCR complex either in a stimulatory way, or in an inhibitory way. Primary signaling sequences that act in a stimulatory manner may contain signaling motifs which are known as immunoreceptor tyrosine-based activation motifs or ITAMs. In some embodiments, the CARs described herein comprise one or more ITAMs.

Examples of ITAM containing primary signaling sequences that are of particular use in the invention include those derived from TCRζ, FcRγ, FERβ, CD3γ, CD3δ, CD3ε, CD3ζ, CD5, CD22, CD79a, CD79b, and CD66d.

In some embodiments, the CAR comprises a primary signaling sequence derived from CD3ζ. For example, the intracellular signaling domain of the CAR can comprise the CD3ζ intracellular signaling sequence by itself or combined with any other desired intracellular signaling sequence(s) useful in the context of the CAR disclosed herein. In some embodiments, the intracellular signaling domain of a CAR provided herein comprises a CD3ζ primary intracellular signaling sequence and a 4-1 BB costimulatory signaling sequence (e.g., the amino acid sequence of SEQ ID NO: 20).

The CARs provided herein may include additional elements, such as a signal peptide to ensure proper export of the fusion protein to the cells surface, a leader sequence (e.g., CD8 leader sequence), and a hinge domain (e.g., CD8 hinge domain) that imparts flexibility to the recognition region and allows strong binding to the targeted moiety. In some embodiments, a spacer domain may be present between any of the domains of the CAR. The spacer domain can be any polypeptide that functions to link two parts of the CAR. A spacer domain may comprise up to about 300 amino acids, including for example about 5 to about 200, about 10 to about 100, or about 25 to about 50 amino acids. Methods of identifying and selecting suitable spacer domains are known in the art.

### V. Antibody Expression and Purification, Nucleic Acids, Vectors, and Cells

The SDC1 antibodies and antigen binding fragments thereof and molecules comprising such antibodies and antigen binding fragments thereof discussed above (e.g., CARs) may be produced by recombinant expression in a human or non-human cell. Antibody-producing cells include non-human cells expressing heavy chains, light chains, or both heavy and light chains; human cells that are not immune cells expressing heavy chains, light chains, or both heavy and light chains; and human B cells that produce heavy chains or light chains, but not both heavy and light chains. The antibodies and antigen binding fragments thereof of this disclosure may be heterologously expressed, in vitro or in vivo, in cells other than human B cells, such as non-human cells and human cells other than B cells, optionally other than immune cells, and optionally in cells other than cells in a B cell lineage.

The SDC1 antibodies and antigen binding fragments thereof and molecules comprising them described herein can be produced using a variety of techniques known in the art of molecular biology and protein chemistry. For example, a nucleic acid encoding the antibody or antigen binding fragment thereof can be inserted into an expression vector that contains transcriptional and translational regulatory sequences, which include, e.g., promoter sequences, ribosomal binding sites, transcriptional start and stop sequences, translational start and stop sequences, transcription terminator signals, polyadenylation signals, and enhancer or activator sequences. The regulatory sequences include a promoter and transcriptional start and stop sequences. In addition, the expression vector can include more than one replication system, such that it can be maintained in two different organisms, for example, in mammalian or insect cells for expression and in a prokaryotic host for cloning and amplification.

Several possible vector systems are available for the expression of cloned heavy chain and light chain polypeptides from nucleic acids in mammalian cells. One class of vectors relies upon the integration of the desired gene sequences into the host cell genome. Cells that have stably integrated DNA can be selected by simultaneously introducing drug resistance genes such as E. coli gpt (Mulligan & Berg, 1981, Proc. Natl. Acad. Sci. USA 78:2072) or Tn5 neo (Southern and Berg, 1982, Mol. Appl. Genet. 1:327). The selectable marker gene can be either linked to the DNA gene sequences to be expressed or introduced into the same cell by co-transfection (Wigler et al., 1979, Cell 16:77). A second class of vectors utilizes DNA elements that confer autonomously replicating capabilities to an extrachromosomal plasmid. These vectors can be derived from animal viruses, such as bovine papillomavirus (Sarver et al., 1982, Proc. Natl. Acad. Sci. USA, 79:7147), CMV, polyoma virus (Deans et al., 1984, Proc. Natl. Acad. Sci. USA 81:1292), or SV40 virus (Lusky & Botchan, 1981, Nature 293:79).

The expression vectors can be introduced into cells in a manner suitable for subsequent expression of the nucleic acid. The method of introduction is largely dictated by the targeted cell type, discussed below. Exemplary methods include CaPO₄ precipitation, liposome fusion, cationic liposomes, electroporation, nucleoporation, viral infection, dextran-mediated transfection, polybrene-mediated transfection, protoplast fusion, and direct microinjection.

Appropriate host cells for the expression of antibodies or antigen binding fragments thereof include yeast, bacteria, insect, plant, and mammalian cells. Of particular interest are bacteria such as E. coli, fungi such as Saccharomyces cerevisiae and Pichia pastoris, insect cells such as SF9, mammalian cell lines (e.g., human cell lines), as well as primary cell lines.

In some embodiments, an antibody or fragment thereof can be expressed in, and purified from, transgenic animals (e.g., transgenic mammals). For example, an antibody can be produced in transgenic non-human mammals (e.g., rodents) and isolated from milk as described in, e.g., Houdebine, 2002, Curr. Opin. Biotechnol. 13 (6): 625-29: van Kuik-Romeijn et al., 2000, Transgenic. Res. 9 (2): 155-59; and Pollock et al., 1999, J. Immunol. Methods 231 (1-2): 147-57.

The antibodies and fragments thereof can be produced from the cells by culturing a host cell transformed with the expression vector containing nucleic acid encoding the antibodies or fragments, under conditions, and for an amount of time, sufficient to allow expression of the proteins. Such conditions for protein expression vary with the choice of the expression vector and the host cell and are easily ascertained by one skilled in the art through routine experimentation. For example, antibodies expressed in E. coli can be refolded from inclusion bodies (see. e.g., Hou et al., 1998, Cytokine 10:319-30). Bacterial expression systems and methods for their use are known in the art (see Ausubel et al., 1988, Current Protocols in Molecular Biology, Wiley & Sons; and Green and Sambrook, 2012, Molecular Cloning—A Laboratory Manual, 4th Ed., Cold Spring Harbor Laboratory Press, New York (2001)). The choice of codons, suitable expression vectors, and suitable host cells vary depending on a number of factors, and may be easily optimized as needed. An antibody (or fragment thereof) described herein can be expressed in mammalian cells or in other expression systems including but not limited to yeast, baculovirus, and in vitro expression systems (see. e.g., Kaszubska et al., 2000, Protein Expression and Purification 18:213-20). Additional discussion of expression vectors for use in eukaryotic cells (e.g., for treating a subject with cancer), along with suitable delivery systems, is provided in Section VIII.A, below.

Also provided herein are nucleic acid molecules encoding an SDC1 antibody or antigen binding portion thereof that binds specifically to SDC1 as described in this disclosure. In some embodiments, the nucleic acid molecules encode an SDC1 antibody or antigen binding fragment thereof comprising a heavy chain variable region comprising an amino acid sequence that is at least 90% identical (for example, at least 90%, 91%, 92%, 93%, 94%, 95%, 96%, 97%, 98%, or 99% identical) to SEQ ID NO: 1 and a light chain variable region comprising an amino acid sequence that is at least 90% identical to SEQ ID NO: 2. In some embodiments, the nucleic acid encodes an isolated antibody or antibody fragment comprising a heavy chain variable region (VH) having at least 90% identity to SEQ ID NO: 1 and comprising a VHCDR1 amino acid sequence comprising SEQ ID NO: 3, a VHCDR2 amino acid sequence comprising SEQ ID NO: 4, and a VHCDR3 amino acid sequence comprising SEQ ID NO: 5, and a light chain variable region (VL) having at least 90% identity to SEQ ID NO: 2 and comprising a VLCDR1 amino acid sequence comprising SEQ ID NO: 6, a VLCDR2 amino acid sequence comprising SEQ ID NO: 7, and a VLCDR3 amino acid sequence comprising SEQ ID NO: 8.

In some embodiments, provided are nucleic acid molecules encoding antibodies or antigen binding fragments thereof that bind specifically to SDC1, wherein the nucleic acid sequences comprise sequences encoding an amino acid sequence that is at least 90% identical (for example, at least 90%, 91%, 92%, 93%, 94%, 95%, 96%, 97%, 98% or 99% identical) to any of the sequences in Table 1.

In some embodiments, provided are nucleic acid molecules comprising a nucleotide sequence that is at least 90% identical (for example, at least 90%, 91%, 92%, 93%, 94%, 95%, 96%, 97%, 98% or 99% identical) to SEQ ID NO: 9 or SEQ ID NO: 10. In some embodiments, the nucleic acid molecules comprise sequences that are at least 90% identical (for example, at least 90%, 91%, 92%, 93%, 94%, 95%, 96%, 97%, 98% or 99% identical) to SEQ ID NO: 9 or SEQ ID NO: 10 and that encode an antibody or antibody fragment that comprises a heavy chain variable region of SEQ ID NO: 1; and a light chain variable of SEQ ID NO: 2.

The amino acid sequences of the CDRs and framework regions can be determined using various well-known definitions in the art, e.g., Kabat, Chothia, international ImMunoGeneTics database (IMGT), AbM, and observed antigen contacts (“Contact”). In some embodiments, CDRs are determined according to the IMGT definition. See, Brochet et al., 2008, Nucl. Acids Res. 36: W503-508. In some embodiments, CDRs are determined by a combination of Kabat, Chothia, and/or Contact CDR definitions.

Also provided herein are DNA constructs comprising a promoter that drives expression in a host cell operably linked to a recombinant nucleic acid molecule comprising a nucleotide sequence that encodes an SDC1 specific antibody or antigen binding fragment thereof.

Also provided herein are vectors, comprising a DNA construct comprising a promoter that drives expression in a host cell operably linked to a recombinant nucleic acid molecule comprising a nucleotide sequence that encodes an SDC1 specific antibody or antigen binding fragment thereof.

Preferred promoters controlling transcription from vectors in mammalian host cells may be obtained from various sources, for example, the genomes of viruses such as polyoma, Simian Virus 40 (SV40), adenovirus, retroviruses, hepatitis B virus, and most preferably cytomegalovirus (CMV), or from heterologous mammalian promoters (e.g., β-actin promoter or EF1α promoter), or from hybrid or chimeric promoters (e.g., CMV promoter fused to the β-actin promoter). Promoters from the host cell or related species are also useful herein.

The term “enhancer” generally refers to a sequence of DNA that functions at no fixed distance from the transcription start site and can be either 5′ or 3′ to the transcription unit. Furthermore, enhancers can be within an intron as well as within the coding sequence itself. They are usually between 10 and 300 bp in length, and they function in cis. Enhancers usually function to increase transcription from nearby promoters. Enhancers can also contain response elements that mediate the regulation of transcription. While many enhancer sequences are known from mammalian genes (globin, elastase, albumin, fetoprotein, and insulin), typically one will use an enhancer from a eukaryotic cell virus for general expression. Preferred examples are the SV40 enhancer on the late side of the replication origin, the cytomegalovirus early promoter enhancer, the polyoma enhancer on the late side of the replication origin, and adenovirus enhancers.

The promoter and/or the enhancer can be inducible (e.g., chemically or physically regulated). A chemically regulated promoter and/or enhancer can, for example, be regulated by the presence of alcohol, tetracycline, a steroid, or a metal. A physically regulated promoter and/or enhancer can, for example, be regulated by environmental factors, such as temperature and light. Optionally, the promoter and/or enhancer region can act as a constitutive promoter and/or enhancer to maximize the expression of the region of the transcription unit to be transcribed. In certain vectors, the promoter and/or enhancer region can be active in a cell type specific manner. Optionally, in certain vectors, the promoter and/or enhancer region can be active in all eukaryotic cells, independent of cell type. Preferred promoters of this type are the CMV promoter, the SV40 promoter, the beta-actin promoter, the EF1A promoter, and the retroviral long terminal repeat (LTR).

The vectors also can include, for example, origins of replication and/or markers. A marker gene can confer a selectable phenotype, e.g., antibiotic resistance, on a cell. The marker product is used to determine if the vector has been delivered to the cell and once delivered is being expressed. Examples of selectable markers for mammalian cells are dihydrofolate reductase (DHFR), thymidine kinase, neomycin, neomycin analog G418, hygromycin, puromycin, and blasticidin. When such selectable markers are successfully transferred into a mammalian host cell, the transformed mammalian host cell can survive if placed under selective pressure. Examples of other markers include, for example, the E. coli lacZ gene, green fluorescent protein (GFP), and luciferase. In addition, an expression vector can include a tag sequence designed to facilitate manipulation or detection (e.g., purification or localization) of the expressed polypeptide. Tag sequences, such as GFP, glutathione S-transferase (GST), polyhistidine, c-myc, hemagglutinin, or FLAG™ tag (Kodak: New Haven, CT) sequences typically are expressed as a fusion with the encoded polypeptide. Such tags can be inserted anywhere within the polypeptide including at either the carboxyl or amino terminus.

Also provided herein are host cells, including bacterial host cells and eukaryotic host cells, comprising a recombinant nucleic acid molecule encoding an SDC1 antibody or antigen binding fragment thereof as described in this disclosure. In some embodiments, the nucleic acid molecule encodes a heavy chain variable region sequence that is at least 90% identical to SEQ ID NO: 9. In some embodiments, the nucleic acid molecule encodes a light chain variable region that is at least 90% identical (for example, at least 90%, 91%, 92%, 93%, 94%, 95%, 96%, 97%, 98% or 99% identical) to SEQ ID NO: 10 and a heavy chain variable region that is at least 90% identical to SEQ ID NO: 9.

Also provided herein are host cells that have been engineered to express and secrete an SDC1 antibody or antigen binding fragment thereof as described in this disclosure. In some embodiments, the cells are suitable for implanting in a patient with cancer. In some embodiments, the cells are animal or human cells, and can be autologous, heterologous, or xenogeneic. In certain embodiments, the cells can be immortalized. In certain embodiments, in order to decrease the chance of an immunological response, the cells can be encapsulated to avoid infiltration of surrounding tissues. In certain embodiments, the encapsulation materials are typically biocompatible, semi-permeable polymeric enclosures or membranes that allow the release of the protein product(s) but prevent the destruction of the cells by a subject's immune system or by other detrimental factors from the surrounding tissues.

Also provided herein are immune cells (e.g., T cells) expressing any of the CARs described herein. In some embodiments, the immune cell expresses the CAR on its surface. In some embodiments, the immune cell comprises a nucleic acid encoding the CAR, wherein the CAR is expressed from the nucleic acid and localized to the immune cell surface. In some embodiments, the immune cell is a B-lymphocyte, T-lymphocyte, thymocyte, dendritic cell, natural killer (NK) cell, monocyte, macrophage, granulocyte, eosinophil, basophil, neutrophil, myelomonocytic cell, megakaryocyte, peripheral blood mononuclear cell, myeloid progenitor cell, or a hematopoietic stem cell. In some embodiments, the immune cell is a T cell. In some embodiments, the T cell is a cytotoxic T cell, a helper T cell, a natural killer T cell, a suppressor T cell, a CD8⁺ T cell, a CD4⁺ T cell, a CD8⁺/CD4⁺ T cell, γδ T cell, or a T-regulatory (T-reg) cell.

In some embodiments, immune cells expressing a CAR provided herein are obtained from a subject. Where the immune cells are used to treat (e.g., according to the treatment methods described herein below) the same subject from which they are obtained, they are referred to as autologous cells. Where they are obtained from a different subject, they are referred to as heterologous cells. Immune cells can be isolated from peripheral blood using techniques well known in the art, include Ficoll density gradient centrifugation followed by negative selection to remove undesired cells. In some embodiments, heterologous immune cells useful for the methods provided herein comprise allogeneic T cells, as described in, e.g., Bedoya et al., 2021, Front. Immunol. 12:640082.

In vitro methods are also suitable for preparing monovalent antibodies or antigen binding fragments thereof. Digestion of antibodies to produce fragments thereof, particularly, Fab fragments, can be accomplished using routine techniques known in the art. For instance, digestion can be performed using papain. Examples of papain digestion are described in International Application Publication No. WO 94/29348, U.S. Pat. No. 4,342,566, and Harlow and Lane, Antibodies, A Laboratory Manual, Cold Spring Harbor Publications, New York, (1988). Papain digestion of antibodies typically produces two identical antigen binding fragments, called Fab fragments, each with a single antigen binding site, and a residual Fc fragment. Pepsin treatment yields a fragment, called the F(ab)₂ fragment that has two antigen combining sites and is still capable of cross-linking antigen.

The Fab fragments produced in antibody digestion can also contain the constant domains of the light chain and the first constant domain of the heavy chain. Fab fragments differ from Fab fragments by the addition of a few residues at the carboxy terminus of the heavy chain domain including one or more cysteines from the antibody hinge region. The F(ab′)₂ fragment is a bivalent fragment comprising two Fab′ fragments linked by a disulfide bridge at the hinge region. Fab-SH is the designation herein for Fab′ in which the cysteine residue(s) of the constant domains bear a free thiol group.

One method of producing proteins comprising the provided antibodies or fragments is to link two or more peptides or polypeptides together by protein chemistry techniques. For example, peptides or polypeptides can be chemically synthesized using currently available laboratory equipment using either Fmoc (9-fluorenylmethyl-oxycarbonyl) or Boc (tert-butyloxy carbonoyl) chemistry (Applied Biosystems, Inc.: Foster City, CA). Those of skill in the art readily appreciate that a peptide or polypeptide corresponding to the antibody provided herein, for example, can be synthesized by standard chemical reactions. For example, a peptide or polypeptide can be synthesized and not cleaved from its synthesis resin whereas the other fragment of an antibody can be synthesized and subsequently cleaved from the resin, thereby exposing a terminal group that is functionally blocked on the other fragment. By peptide condensation reactions, these two fragments can be covalently joined via a peptide bond at their carboxyl and amino termini, respectively, to form an antibody, or fragment thereof. (Grant GA, 1992, Synthetic Peptides: A User Guide. W.H. Freeman and Co., N.Y.; Bodansky M and Trost B., Ed., 1993, Principles of Peptide Synthesis. Springer Verlag Inc., NY). Alternatively, the peptide or polypeptide can by independently synthesized in vivo. Once isolated, these independent peptides or polypeptides may be linked to form an antibody or fragment thereof via similar peptide condensation reactions.

For example, enzymatic ligation of cloned or synthetic peptide segments can allow relatively short peptide fragments to be joined to produce larger peptide fragments, polypeptides, or whole protein domains (Abrahmsen et al., 1991, Biochemistry, 30:4151). Alternatively, native chemical ligation of synthetic peptides can be utilized to synthetically construct large peptides or polypeptides from shorter peptide fragments. This method consists of a two-step chemical reaction (Dawson et al., 1994, Science, 266:776 779). The first step is the chemoselective reaction of an unprotected synthetic peptide a thioester with another unprotected peptide segment containing an amino terminal Cys residue to give a thioester linked intermediate as the initial covalent product. Without a change in the reaction conditions, this intermediate undergoes spontaneous, rapid intramolecular reaction to form a native peptide bond at the ligation site. Application of this native chemical ligation method to the total synthesis of a protein molecule is illustrated by the preparation of human interleukin 8 (IL-8) (Baggiolini et al., 1992, FEBS Lett. 307:97-101; Clark et al., 1994, J. Biol. Chem. 269:16075; Clark et al., 1991, Biochemistry 30:3128; Rajarathnam et al., 1994, Biochemistry 33:6623-30).

Alternatively, unprotected peptide segments can be chemically linked where the bond formed between the peptide segments as a result of the chemical ligation is an unnatural (non-peptide) bond (Schnolzer et al., 1992, Science 256:221). This technique has been used to synthesize analogs of protein domains as well as large amounts of relatively pure proteins with full biological activity (deLisle et al., 1992, Techniques in Protein Chemistry IV. Academic Press, New York, pp. 257-267).

Following expression, the antibodies and fragments thereof can be isolated. An antibody or fragment thereof can be isolated or purified in a variety of ways known in the art depending on what other components are present in the sample. Standard purification methods include electrophoretic, molecular, immunological, and chromatographic techniques, including ion exchange, hydrophobic, affinity, and reverse-phase HPLC chromatography. For example, an antibody can be purified using a standard anti-antibody column (e.g., a protein-A or protein-G column). Ultrafiltration and diafiltration techniques, in conjunction with protein concentration, are also useful. See. e.g., Scopes, 1994, Protein Purification, 3^{rd} edition, Springer-Verlag, New York City, New York. The degree of purification necessary varies depending on the desired use. In some instances, no purification of the expressed antibody or fragments thereof is necessary.

Methods for determining the yield or purity of a purified antibody or fragment thereof are known in the art and include, e.g., Bradford assay, UV spectroscopy, Biuret protein assay, Lowry protein assay, amido black protein assay, high pressure liquid chromatography (HPLC), mass spectrometry (MS), and gel electrophoretic methods (e.g., using a protein stain such as Coomassie Blue or colloidal silver stain).

### VI. SDC1-Targeted Theranostic Pair

In one aspect, the present disclosure provides a theranostic pair that bind specifically to SDC1. In some embodiments, the theranostic pair comprises a first radiolabeled antibody comprising a syndecan 1 (SDC1) antibody or antibody fragment thereof conjugated to a first chelating agent and a first radioactive moiety that is an imaging agent; and a second radiolabeled antibody comprising the SDC1 antibody or antibody fragment thereof conjugated to a second chelating agent and a second radioactive moiety that is a therapeutic agent.

### SDC1-Targeted Antibody

The present disclosure provides radionuclide conjugated antibodies that bind specifically to SDC1. Antibodies and antigen binding fragments can be described by the antigen to which they specifically bind. For example, as used herein, the terms “SDC1 radionuclide conjugated antibody” and “anti-SDC1 antibody conjugated to a radioactive moiety” both refer to an antibody, conjugated to a radiolabel, that specifically binds SDC1. Acceptable antibodies for use in SDC1-targeted theranostic pair described herein are described in Section III, above.

Provided herein are theranostic pairs comprising antibodies and antigen binding portions thereof that bind specifically to SDC1. The SDC1 antibodies and antigen binding portions thereof are polypeptides. As used herein, the terms “antigen binding portion,” “antigen-binding fragment,” and “antibody fragment” are used interchangeably to refer to a portion of an antibody polypeptide sequence that binds specifically to SDC1.

In one aspect, provided herein is a theranostic pair comprising an isolated antibody or antibody fragment, wherein the antibody or antibody fragment comprises: a heavy chain variable region (VH) having at least 90% identity (for example, at least 90%, 91%, 92%, 93%, 94%, 95%, 96%, 97%, 98%, or 99% identical) to SEQ ID NO: 1 and comprising a CDRH1 amino acid sequence comprising SEQ ID NO: 3, a CDRH2 amino acid sequence comprising SEQ ID NO: 4, and a CDRH3 amino acid sequence comprising SEQ ID NO: 5; and a light chain variable region (VL) having at least 90% identity (for example, at least 90%, 91%, 92%, 93%, 94%, 95%, 96%, 97%, 98% or 99% identical) to SEQ ID NO: 2 and comprising a CDRL1 amino acid sequence comprising SEQ ID NO: 6, a CDRL2 amino acid sequence comprising SEQ ID NO: 7, and a CDRL3 amino acid sequence comprising SEQ ID NO: 8.

In some embodiments, heavy chain variable region sequences and light chain variable region sequences encompassed by the antibody of the theranostic pair of this disclosure are set forth in Table 1. The CDR sequences in the variable domains listed in Table 1 are indicated by bold and underlined text. In some embodiments, the heavy chain variable region is encoded by a nucleotide sequence having at least 90% identity to SEQ ID NO: 9. In some embodiments, the light chain variable region is encoded by a nucleotide sequence having at least 90% identity to SEQ ID NO: 10.

The disclosure also provides a theranostic pair comprising an antibody or antigen binding portion thereof that specifically binds to SDC1, wherein the antibody or antigen binding portion thereof comprises a heavy chain variable region comprising an amino acid sequence that is at least 90% identical (for example, at least 90%, 91%, 92%, 93%, 94%, 95%, 96%, 97%, 98% or 99% identical) to SEQ ID NO: 1 and a light chain variable region comprising an amino acid sequence that is at least 90% identical (for example, at least 90%, 91%, 92%, 93%, 94%, 95%, 96%, 97%, 98% or 99% identical) to SEQ ID NO: 2. Table 1 provides the sequences for SEQ ID NOs: 1 and 2.

In each case, where a specific amino acid sequence is recited, embodiments comprising a sequence having at least 90% (e.g., 90%, 91%, 92%, 93%, 94%, 95%, 96%, 97%, 98%, or 99%) identity to the recited sequence are also provided.

In some embodiments, a humanized monoclonal antibody comprises CDR sequences, a heavy chain variable region, and/or a light chain variable region as described herein (e.g., as disclosed in Table 1) and further comprises a heavy chain constant region and/or a light chain constant region that is heterologous to the antibody produced by the methods described herein and, in the Examples, below from which the CDR sequences and/or variable region sequences are derived. For example, in some embodiments, the monoclonal antibody comprises the CDR sequences and/or variable region sequences of an antibody described herein, and further comprises a heavy chain constant region and a light chain constant region that is heterologous to the antibody described herein (e.g., the heavy chain constant region and/or light chain constant region is a wild-type or modified IgG1, IgG2, IgG3, or IgG4 constant region), or the heavy chain constant region and/or light chain constant region comprises one or more modifications (e.g., amino acid substitutions) relative to the native constant region of the antibodies described herein.

The antibodies and fragments thereof of this disclosure may comprise variations in heavy chain constant regions to change the properties of the synthetic antibody relative to the corresponding naturally occurring antibody. An exemplary change includes mutations to alter half-life. See Fonseca et al., 2018, “Boosting half-life and effector functions of therapeutic antibodies by Fc-engineering: An interaction-function review” Int. J. Biol. Macromol. 19:306-11.

In some embodiments, the 22B antibodies, used in the radiolabeled conjugate antibody described herein, can be modified, e.g., with a moiety that improves the stabilization and/or retention of the antibodies in circulation, e.g., in blood, serum, or other tissues. For example, the antibody or fragment can be PEGylated as described in, e.g., Lee et al., 1999, Bioconjug. Chem. 10 (6): 973-78: Kinstler et al., 2002, Advanced Drug Deliveries Reviews 54:477-485; and Roberts et al., 2002, Advanced Drug Delivery Reviews 54:459-476, or HESylated (Fresenius Kabi, Germany) (see. e.g., Pavisić et al., 2010, Int. J. Pharm. 387 (1-2): 110-119). The stabilization moiety can improve the stability, or retention of, the antibody (or fragment) by at least 1.5 (e.g., at least 2, 5, 10, 15, 20, 25, 30, 40, or 50 or more) fold.

In some embodiments, the antibodies or antigen-binding fragments thereof described herein can be glycosylated. In some embodiments, an antibody or antigen-binding fragment thereof described herein can be subjected to enzymatic or chemical treatment, or produced from a cell, such that the antibody or fragment has reduced or absent glycosylation. Methods for producing antibodies with reduced glycosylation are known in the art and described in, e.g., U.S. Pat. No. 6,933,368; Wright et al., 1991, EMBO J. 10 (10): 2717-2723; and Co et al., (1993), Mol. Immunol. 30:1361.

### Radiolabels

The radiolabel conjugated to the SDC1-specific antibody in the theranostic pair may be selected, for example, from a group consisting of ²⁷⁷Ac, ²¹¹At, ¹²⁸Ba, ¹³¹Ba, ⁷Be, ²⁰⁴Bi, ²⁰⁵Bi, ²⁰⁶Bi, ⁷⁶Br, ⁷⁷Br, ⁸²Br, ¹⁰⁹Cd, ⁴⁷Ca, ¹¹C, ¹⁴C, ³⁶Cl, ⁴⁸Cr, ⁵¹Cr, ⁶²Cu, ⁶⁴Cu, ⁶⁷Cu, ¹⁶⁵Dy, ¹⁵⁵Eu, ¹⁸F, ¹⁵³Gd, ⁶⁶Ga, ⁶⁷Ga, ⁶⁸Ga, ⁷²Ga, ¹⁹⁸Au, ³H ¹⁶⁶Ho, ¹¹¹In, ^{113m}In, ^{115m}In, ¹²³I, ¹²⁵I, ¹³¹I, ¹⁸⁹Ir, ^{191m}Ir, ¹⁹²Ir, ¹⁹⁴Ir, ⁵²Fe, ⁵⁵Fe, ⁵⁹Fe, ¹⁷⁷Lu, ¹⁵O, ^{191m-191}Os, ¹⁰⁹Pd, ³²P, ³³P, ⁴²K, ²²⁶Ra, ¹⁸⁶Re, ¹⁸⁸Re, ^{82m}Rb, ¹⁵³Sm, ⁴⁶Sc, ⁴⁷Sc, ⁷²Se, ⁷⁵Se, ¹⁰⁵Ag, ²²Na, ²⁴Na, ⁸⁹Sr, ³⁵S, ³⁸S, ¹⁷⁷Ta, ⁹⁶Tc, ^{99m}Tc, ²⁰¹Tl, ²⁰²Tl, ¹¹³Sn, ^{117m}Sn, ¹²¹Sn, ¹⁶⁶Yb, ¹⁶⁹Yb, ¹⁷⁵Yb, ⁸⁸Y, ⁹⁰Y, ⁶²Zn, ⁶⁵Zn, ⁸⁹Zr. In some embodiments, the radioactive moiety is selected from a group consisting of ¹⁶¹Tb, ²²⁵Ac, ¹⁶¹Tb/²²⁵Ac, ⁸⁹Zr, ¹⁷⁷Lu, ¹³⁴Ce, ¹⁴⁰Nd, ¹⁶⁹Er, ¹³⁴Ce/¹³⁴La, and ¹⁴⁰Nd/¹⁴⁰Pr. In some embodiments, the radioisotope is ⁸⁹Zr, ¹³¹I, ¹²⁵I, ¹²³I, ¹¹¹I, ^{99m}Tc, ⁹⁰Y, ¹⁸⁶Re, ¹⁸⁸Re, ³²P, ¹⁵³Sm, ⁶⁷Ga, ²⁰¹Tl, ⁷⁷Br, or ¹⁸F. In some embodiments, more than one radiolabeled antibody may be produced according to methods that are generally known in the art. For example, the radiolabeled antibodies as described herein may include an SDC antibody conjugated to [⁸⁹Zr], [¹⁶¹Tb], or [²²⁵Ac]. In some embodiments, a theranostic pair is used in the methods described below, which may include administering a first radiolabeled antibody for imaging a tumor in a subject and subsequently administering a second radiolabeled antibody for treatment of a subject. In some embodiments, the [⁸⁹Zr], [¹⁶¹Tb], and [²²⁵Ac] radiolabeled antibodies may independently be used for imaging and therapeutic treatment of a subject with cancer. In some embodiments, the first radiolabeled antibody and second radiolabeled antibody may include the same radiolabel (e.g., both [⁸⁹Zr]). In some embodiments, the radiolabel for each antibody in the theranostic pair is different. In some embodiments, the first radiolabeled antibody in the theranostic pair is a [⁸⁹Zr] labeled SDC1 antibody and the second radiolabeled antibody in the theranostic pair is a [¹⁶¹Tb] labeled SDC1 antibody. Such procedures for labeling biological agents with the radioactive isotopes are generally known in the art.

The SDC1 radiolabeled antibodies as described herein may rapidly internalize in a SDC1+ tissue. Given the rapid internalization characteristics as described further below, more than one radiolabeled antibody may be generated as described above. The different isotopes used in the radiolabeled antibody for treatment may individually have unique mechanisms of damage. For example, a [¹⁶¹Tb]-labeled antibody may have a dual emission of medium energy beta particles and Auger electrons. Conversely, a [²²⁵Ac]-labeled antibody may emit high linear alpha particles. In some embodiments, a combined treatment including a [¹⁶¹Tb]-labeled antibody and a [²²⁵Ac]-labeled antibody may produce a competitive environment of the beta and alpha particles with the Auger electrons, potentially reducing toxicity of the radiolabeled antibody treatment options. In some embodiments, the radiolabeled antibodies described herein may be generated at least in part based on the type of particle and electron emission from the isotopes. In some embodiments, treatment plans may be generated based on the emitted particles from the isotopes. Thus, described herein may include patient specific and tumor specific radiolabeled antibodies and methods of treating such cancer.

Techniques for conjugating a therapeutic moiety (e.g., a radioactive moiety) to an SDC1-specific antibody or antigen binding fragment thereof are well known. See, for example, Arnon et al., 1985, Monoclonal Antibodies And Cancer Therapy, Reisfeld et al. (eds.), pp. 243-56; Hellstrom et al., 1987, Controlled Drug Delivery (2nd Ed.), Robinson et al. (eds.), pp. 623-53; Thorpe, 1985, Monoclonal Antibodies '84: Biological And Clinical Applications, Pinchera et al. (eds.), pp. 475-506: “Analysis, Results, And Future Prospective Of The Therapeutic Use Of Radiolabeled Antibody In Cancer Therapy” In: Monoclonal Antibodies For Cancer Detection And Therapy, (Baldwin et al. eds.), pp. 303-316 (1985), and Thorpe et al., 1982, Immunol. Rev. 62:119-158.

In some embodiments, a radioactive label can be directly conjugated to the amino acid backbone of the antibody. Alternatively, the radioactive label can be included as part of a larger molecule (e.g., ¹²⁵I in meta-[¹²⁵]iodophenyl-N-hydroxysuccinimide ([¹²⁵I]mIPNHS), which binds to free amino groups to form meta-iodophenyl (mIP) derivatives of relevant proteins (see. e.g., Rogers et al., 1997, J. Nucl. Med. 38:1221-29) or chelate (e.g., to DOTA or DTPA), which is in turn bound to the protein backbone. Methods of conjugating the radioactive labels, or larger molecules/chelates containing them, to the antibodies or antigen binding fragments are known in the art. Such methods involve incubating the proteins with the radioactive label under conditions (e.g., pH, salt concentration, and/or temperature) that facilitate binding of the radioactive label or chelating agent to the protein (see. e.g., U.S. Pat. No. 6,001,329). In some embodiments, chelating agents may include diethylene triamine pentaacetic acid (DTPA) or tetraazacyclododecane-1,4,7,10-tetraacetic acid (DOTA), deferoxamine (DFO), or 1,4,7,10-tetraazacyclododecane (DO3A).

In some embodiments, the SDC1-specific radiolabeled antibodies described herein may include a linking group that may bind specifically to the amino acids of the antibody and further bind to the chelating agent. Methods of conjugating the chelating group for radioactive labeling are known in the art.

### VII. Pharmaceutical Compositions and Formulations

The SDC1 antibodies and antigen binding portions thereof described herein, as well as the various molecules comprising said antibodies and antigen binding portions thereof (e.g., CARs) are suitable for administration in vitro or in vivo. In some embodiments, the compositions comprise an SDC1 antibody or antigen binding fragment thereof of the present disclosure and a pharmaceutically acceptable carrier (excipient). In some embodiments, the compositions comprise a CAR comprising the SDC1 antibody or antigen binding fragment thereof. A pharmaceutically acceptable carrier (excipient) is a material that is not biologically or otherwise undesirable, i.e., the material is administered to a subject without causing undesirable biological effects or interacting in a deleterious manner with the other components of the pharmaceutical composition in which it is contained. The carrier is selected to minimize any degradation of the active ingredient and to minimize any adverse side effects in the subject. The compositions may further comprise a diluent, solubilizer, emulsifier, preservative, and/or adjuvant to be used with the methods disclosed herein. Such compositions can be used, for example, in a subject with cancer that would benefit from any of the SDC1 antibodies or antigen binding fragments thereof or molecules comprising the SDC1-specific antibody or antigen binding fragment thereof as described herein.

The radiolabeled antibodies described herein are suitable for administration in vitro or in vivo. In some embodiments, compositions comprising an SDC1 antibody conjugated to radioactive moiety include a pharmaceutically acceptable carrier (excipient). A pharmaceutically acceptable carrier (excipient) is a material that is not biologically or otherwise undesirable, i.e., the material is administered to a subject without causing undesirable biological effects or interacting in a deleterious manner with the other components of the pharmaceutical composition in which it is contained. The carrier is selected to minimize any degradation of the active ingredient and to minimize any adverse side effects in the subject. The compositions may further comprise a diluent, solubilizer, emulsifier, preservative, and/or adjuvant to be used with the methods disclosed herein. Such compositions can be used, for example, in a subject with cancer that would benefit from any of the SDC1-specific radionuclide conjugated antibodies as described herein.

Suitable carriers and their formulations are described in Remington: The Science and Practice of Pharmacy. 21^{st} Edition, Philip P. Gerbino, ed., Lippincott Williams & Wilkins (2006). In certain embodiments, acceptable formulation materials preferably are nontoxic to recipients at the dosages and concentrations employed. In certain embodiments, the formulation material(s) are for subcutaneous and/or intravenous administration. In certain embodiments, the formulation comprises an appropriate amount of a pharmaceutically-acceptable salt to render the formulation isotonic. In certain embodiments, the pharmaceutical composition can contain formulation materials for modifying, maintaining, or preserving, for example, the pH, osmolality, viscosity, clarity, color, isotonicity, odor, sterility, stability, rate of dissolution or release, adsorption or penetration of the composition. In certain embodiments, suitable formulation materials include, but are not limited to, amino acids (such as glycine, glutamine, asparagine, arginine or lysine); antimicrobials; antioxidants (such as ascorbic acid, sodium sulfite or sodium hydrogen-sulfite); buffers (such as borate, bicarbonate, Tris-HCl, citrates, phosphates or other organic acids); bulking agents (such as mannitol or glycine); chelating agents (such as ethylenediamine tetraacetic acid (EDTA)); complexing agents (such as caffeine, polyvinylpyrrolidone, beta-cyclodextrin or hydroxypropyl-beta-cyclodextrin); fillers: monosaccharides, disaccharides, and other carbohydrates (such as glucose, mannose or dextrins): proteins (such as serum albumin, gelatin or immunoglobulins): coloring, flavoring and diluting agents; emulsifying agents; hydrophilic polymers (such as polyvinylpyrrolidone); low molecular weight polypeptides; salt-forming counterions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, PEG, sorbitan esters, polysorbates such as polysorbate 20, polysorbate 80, triton, tromethamine, lecithin, cholesterol, tyloxapal); stability enhancing agents (such as sucrose or sorbitol); tonicity enhancing agents (such as alkali metal halides, preferably sodium or potassium chloride, mannitol sorbitol): delivery vehicles: diluents: excipients and/or pharmaceutical adjuvants. In certain embodiments, the optimal pharmaceutical composition is determined by one skilled in the art depending upon, for example, the intended route of administration, delivery format and desired dosage. See, for example, Remington: The Science and Practice of Pharmacy, 22^{nd} Edition, Lloyd V. Allen, Jr., ed., The Pharmaceutical Press (2014). In certain embodiments, such compositions may influence the physical state, stability, rate of in vivo release and/or rate of in vivo clearance of the SDC1-specific antibody or antigen binding fragment thereof or molecules comprising the SDC1-specific antibody or antigen binding fragment thereof. In certain embodiments, the SDC1-specific antibody or antigen binding fragment thereof is a SDC1-specific radiolabeled antibody.

In certain embodiments, the primary vehicle or carrier in a pharmaceutical composition can be either aqueous or non-aqueous in nature. For example, in certain embodiments, a suitable vehicle or carrier can be sterile water for injection, physiological saline solution, buffered solutions like Ringer's solution, dextrose solution, or artificial cerebrospinal fluid, possibly supplemented with other materials common in compositions for parenteral administration. In certain embodiments, the saline comprises isotonic phosphate-buffered saline. In certain embodiments, neutral buffered saline or saline mixed with serum albumin are further exemplary vehicles. In certain embodiments, pharmaceutical compositions comprise a pH controlling buffer such phosphate-buffered saline or acetate-buffered saline. In certain embodiments, a composition comprising an SDC1-specific antibody or antigen binding fragment thereof disclosed herein can be prepared for storage by mixing the selected composition having the desired degree of purity with optional formulation agents (see Remington: The Science and Practice of Pharmacy, 22^{nd} Edition, Lloyd V. Allen, Jr., ed., The Pharmaceutical Press (2014)) in the form of a lyophilized cake or an aqueous solution. Further, in certain embodiments, a composition comprising an SDC1-specific antibody or antigen binding fragment thereof disclosed herein can be formulated as a lyophilizate using appropriate excipients. In some instances, appropriate excipients may include a cryo-preservative, a bulking agent, a surfactant, or a combination of any thereof. Exemplary excipients include one or more of a polyol, a disaccharide, or a polysaccharide, such as, for example, mannitol, sorbitol, sucrose, trehalose, and dextran 40. In some embodiments, the cryo-preservative may be sucrose or trehalose. In some embodiments, the bulking agent may be glycine or mannitol. In one example, the surfactant may be a polysorbate such as, for example, polysorbate-20 or polysorbate-80. In certain embodiments, a composition comprising an SDC1-specific antibody conjugated to a radioactive moiety as disclosed herein can be prepared for storage by mixing the selected composition having the desired degree of purity with optional formulation agents. In some embodiments, the radiolabeled antibody as described herein may be shelf stable for up to two months. In some embodiments, the shelf life stability of the radiolabeled antibody may be longer than two months. The stability of the radiolabeled antibody may be dependent upon the half-life of the radiolabel. For example, the half-life of [⁸⁹Zr] may be 80 hours while [¹⁶¹Tb] may have a half-life of about 7 days. Thus, in some embodiments, the radiolabeled antibodies as described herein may be prepared hours or days before use in imaging or treatment.

In certain embodiments, the pharmaceutical composition can be selected for parenteral delivery (e.g., through injection by intravenous, intraperitoneal, intracerebral (intra-parenchymal), intracerebral, intraventricular, intramuscular, subcutaneous, intra-ocular, intraarterial, intraportal, or intralesional routes). Preparations for parenteral administration can be in the form of a pyrogen-free, parenterally acceptable aqueous solution (i.e., water, alcoholic/aqueous solutions, emulsions, or suspensions, including saline and buffered media) comprising an SDC1-specific antibody or antigen binding fragment thereof in a pharmaceutically acceptable vehicle (e.g., an SDC1-specific radiolabeled antibody in a pharmaceutically acceptable vehicle. Preparations for parenteral administration can also include non-aqueous solutions, suspensions, and emulsions. Examples of non-aqueous solvents are propylene glycol, polyethylene glycol, vegetable oils such as olive oil, and injectable organic esters such as ethyl oleate. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives are optionally present such as, for example, antimicrobials, anti-oxidants, chelating agents, and inert gases and the like. In certain embodiments, the preparation can involve the formulation of the desired molecule with an agent, such as injectable microspheres, bio-erodible particles, polymeric compounds (such as polylactic acid or polyglycolic acid), beads or liposomes, that can provide for the controlled or sustained release of the product which can then be delivered via a depot injection. In certain embodiments, hyaluronic acid can also be used, and can have the effect of promoting sustained duration in the circulation. In certain embodiments, implantable drug delivery devices can be used to introduce the desired molecule.

In certain embodiments, the compositions can be selected for inhalation or for delivery through the digestive tract, such as orally. Compositions for oral administration include powders or granules, suspension or solutions in water or non-aqueous media, capsules, sachets, or tables. Thickeners, flavorings, diluents, emulsifiers, dispersing aids, or binders are optionally desirable.

In certain embodiments, the compositions can be selected for topical delivery. Formulations for topical administration include ointments, lotions, creams, gels, drops, suppositories, sprays, liquids, and powders. Conventional pharmaceutical carriers, aqueous, powder, or oily bases, thickeners and the like are optionally necessary or desirable.

In certain embodiments, the formulation components are present in concentrations that are acceptable to the site of administration. In certain embodiments, buffers are used to maintain the composition at physiological pH or at a slightly lower pH, typically within a pH range of from about 5 to about 8. For example, the pH may be 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8. 6.9, 6.9, 7.0, 7.1, 7.2, 7.3, 7.4, 7.5, 7.6, 7.7, 7.8, 7.9, 8.0, 8.1, 8.2, 8.3, 8.4, or 8.5. In some instances, the pH of the pharmaceutical composition may be in the range of 6.6-8.5 such as, for example, 7.0-8.5, 6.6-7.2, 6.8-7.2, 6.8-7.4, 7.2-7.8, 7.0-7.5, 7.5-8.0, 7.2-8.2, 7.6-8.5, or 7.8-8.3. In some instances, the pH of the pharmaceutical composition may be in the range of 5.5-7.5 such as, for example, 5.5-5.8, 5.5-6.0, 5.7-6.2, 5.8-6.5, 6.0-6.5, 6.2-6.8, 6.5-7.0, 6.8-7.2, or 6.8-7.5. In some instances, the pH of the pharmaceutical composition may be in the range of 4.0-5.5 such as, for example, 4.0-4.3, 4.0-4.5, 4.2-4.8, 4.5-4.8, 4.5-5.0, 4.8-5.2, or 5.0-5.5.

In certain embodiments, a pharmaceutical composition can comprise an effective amount of an SDC1 antibody or antigen binding fragment thereof in a mixture with non-toxic excipients suitable for the manufacture of tablets. In certain embodiments, a pharmaceutical composition can comprise an effective amount of an SDC1 radiolabeled antibody in a mixture with non-toxic excipients suitable for the manufacture of tablets. In certain embodiments, by dissolving the tablets in sterile water or other appropriate vehicle, solutions can be prepared in unit-dose form. In certain embodiments, suitable excipients include, but are not limited to, inert diluents, such as calcium carbonate, sodium carbonate or bicarbonate, lactose, or calcium phosphate; or binding agents, such as starch, gelatin, or acacia; or lubricating agents such as magnesium stearate, stearic acid, or talc.

Additional pharmaceutical compositions can be selected by one skilled in the art, including formulations involving an SDC1-specific antibody or antigen binding fragment thereof in sustained- or controlled-delivery formulations. In some embodiments, additional pharmaceutical compositions can be selected by one skilled in the art, including formulations involving an SDC1-specific radiolabeled antibody in sustained- or controlled-delivery formulations. In certain embodiments, techniques for formulating a variety of other sustained- or controlled-delivery means, such as liposome carriers, bio-erodible microparticles or porous beads and depot injections, are also known to those skilled in the art. See for example, International Application Publication No. WO1993/015722, which describes the controlled release of porous polymeric microparticles for the delivery of pharmaceutical compositions. In certain embodiments, sustained-release preparations can include semipermeable polymer matrices in the form of shaped articles, e.g., films, or microcapsules. Sustained release matrices can include polyesters, hydrogels, polylactides (see, e.g., U.S. Pat. Nos. 3,773,919; 5,594,091; 8,383,153; 4,767,628; International Application Publication No. WO1998/043615, Calo et al., 2015, Eur. Polymer J. 65:252-67 and European Patent No. EP 058,481), including, for example, chemically synthesized polymers, starch based polymers, and polyhydroxyalkanoates (PHAs), copolymers of L-glutamic acid and gamma ethyl-L-glutamate (Sidman et al., 1993, Biopolymers 22:547-56), poly (2-hydroxyethyl-methacrylate) (Langer et al., 1981, J. Biomed. Mater. Res. 15:167-277; and Langer, 1982, Chem. Tech. 12:98-105), ethylene vinyl acetate (Hsu & Langer, 1985, J. Biomed. Materials Res. 19 (4): 445-60) or poly-D (−)-3-hydroxybutyric acid (European Patent No. EP0133988). In certain embodiments, sustained release compositions can also include liposomes, which can be prepared by any of several methods known in the art. (See, e.g., Eppstein et al., 1985, Proc. Natl. Acad. Sci. USA 82:3688-92: European Patent No. EP 036,676; and U.S. Pat. Nos. 4,619,794 and 4,615,885).

The pharmaceutical composition to be used for in vivo administration typically is sterile. In certain embodiments, sterilization is accomplished by filtration through sterile filtration membranes. In certain embodiments, where the composition is lyophilized, sterilization using this method can be conducted either prior to or following lyophilization and reconstitution. In certain embodiments, the composition for parenteral administration can be stored in lyophilized form or in a solution. In certain embodiments, parenteral compositions generally are placed into a container having a sterile access port, for example, an intravenous solution bag or vial having a stopper pierceable by a hypodermic injection needle.

In certain embodiments, once the pharmaceutical composition has been formulated, it can be stored in sterile vials as a solution, suspension, gel, emulsion, solid, or as a dehydrated or lyophilized powder. In certain embodiments, such formulations can be stored either in a ready-to-use form or in a form (e.g., lyophilized) that is reconstituted prior to administration.

The term “unit dose” or “dosage” refers to physically discrete units suitable for use in a subject, each unit containing a predetermined quantity of the therapeutic composition calculated to produce the desired responses discussed above in association with its administration, i.e., the appropriate route and treatment regimen. The quantity to be administered, both according to number of treatments and unit dose, depends on the effect desired. The actual dosage amount of a composition of the present embodiments administered to a patient or subject can be determined by physical and physiological factors, such as body weight, the age, health, and sex of the subject, the type of disease being treated, the extent of disease penetration, previous or concurrent therapeutic interventions, idiopathy of the patient, the route of administration, and the potency, stability, and toxicity of the particular therapeutic substance. For example, a dose may also comprise from about 1 μg/kg/body weight to about 1000 mg/kg/body weight (this such range includes intervening doses) or more per administration, and any range derivable therein. In non-limiting examples of a derivable range from the numbers listed herein, a range of about 5 μg/kg/body weight to about 100 mg/kg/body weight, about 5 μg/kg/body weight to about 500 mg/kg/body weight, etc., can be administered. The practitioner responsible for administration will, in any event, determine the concentration of active ingredient(s) in a composition and appropriate dose(s) for the individual subject. In certain embodiments, the SDC1-specific antibodies or antigen-binding fragments thereof, or molecules comprising the SDC1-specific antibody or antigen binding fragment thereof, can be administered at a dose of 1 mg/kg. 2 mg/kg, 3 mg/kg, 4 mg/kg, or 5 mg/kg once every other day at least four times. An exemplary treatment regime may include administration once per day, once per week, twice a week, once every two weeks, once every three weeks, once every four weeks, once a month, once every 3 months, or once every three to 6 months. In some cases, the treatment comprises administering SDC1-specific antibodies or antigen-binding fragments thereof, or molecules comprising the SDC1-specific antibody or antigen binding fragment thereof, according to one of the aforementioned dosing regimens for a first period and another of the aforementioned dosing regimens for a second period. In some cases, the treatment discontinues for a period of time before the same or a different dosing regimen resumes. For example, a patient may be on an SDC1-specific antibody dosing regimen for two weeks, off for a week, on for another two weeks, and so on. Dosage regimens for SDC1-specific antibodies or antigen-binding fragments thereof of this disclosure include 0.1 mg/kg body weight, 0.3 mg/kg body weight, 2 mg/kg body weight, 3 mg/kg body weight, or 10 mg/kg via intravenous administration, with the SDC1-specific antibodies or antigen-binding fragments thereof being given using one of the following dosing schedules: (i) every four weeks for six dosages, then every three months; (ii) every three weeks; (iii) 3 mg/kg body weight once followed by 1 mg/kg body weight every three weeks.

In certain embodiments, the SDC1-specific radiolabeled antibodies or molecules comprising the SDC1-specific radiolabeled antibody can be administered at a dose of 1 mg/kg. 2 mg/kg, 3 mg/kg. 4 mg/kg, or 5 mg/kg once every other day at least four times. In some embodiments, SDC1-specific radiolabeled antibodies or molecules comprising the SDC1-specific radiolabeled antibody can be administered at a dose of 1 μCi to 300 μCi (e.g., 1-400, 10-350, 50-300, 100-300, 150-300, 200-300, 100-150, 100-200, 100-250 μCi, etc, and every range within). In some embodiments, the SDC1-specific radiolabeled antibody can be administered at a dose of about 1 μCi to 300 μCi in a single dose, in dose intervals, or over a number of days (e.g., 1 day, 2 days, 3 days, 4 days, or 5 days). In some embodiments, the dose concentration may be dependent upon the conjugated antibody as described above. In some embodiments, an exemplary treatment regime may include administration once per day, once per week, twice a week, once every two weeks, once every three weeks, once every four weeks, once a month, once every 3 months, or once every three to 6 months. In some cases, the treatment comprises administering SDC1-specific radiolabeled antibodies or molecules comprising the SDC1-specific radiolabeled antibody according to one of the aforementioned dosing regimens for a first period and another of the aforementioned dosing regimens for a second period. In some cases, the treatment discontinues for a period of time before the same or a different dosing regimen resumes. For example, a patient may be on an SDC1-specific radiolabeled antibody dosing regimen for two weeks, off for a week, on for another two weeks, and so on. Dosage regimens for SDC1-specific radiolabeled antibodies of this disclosure include 0.1 mg/kg body weight, 0.3 mg/kg body weight, 2 mg/kg body weight, 3 mg/kg body weight, or 10 mg/kg via intravenous administration, with the SDC1-specific radiolabeled antibodies being given using one of the following dosing schedules: (i) every four weeks for six dosages, then every three months; (ii) every three weeks; (iii) 3 mg/kg body weight once followed by 1 mg/kg body weight every three weeks. Dosage regimens for SDC1-specific radiolabeled antibodies of this disclosure include, for example, 0.1 mg/kg body weight 1 μCi, 10 μCi, 20 μCi, 30 μCi, 40 μCi, 50 μCi, 100 μCi, 150 μCi, 200 μCi, 250 μCi, or 300 μCi via intravenous administration (or any value within 1 μCi to 300 μCi), with the SDC1-specific radiolabeled antibodies being given using one of the following dosing schedules: (i) every four weeks for six dosages, then every three months; (ii) every three weeks; (iii) higher dosage once followed by lower dosage every three weeks.

In still another aspect, unit dose forms comprising an SDC1-specific antibody or antigen binding fragment thereof as described in this disclosure (e.g., an SDC1-specific radiolabeled antibody) are provided. A unit dose form can be formulated for administration according to any of the routes described in this disclosure. In one example, the unit dose form is formulated for intravenous or intraperitoneal administration. In still another aspect, pharmaceutical packages comprising unit dose forms of an SDC1-specific antibody or antigen binding fragment thereof, or of molecules comprising the SDC1-specific antibody or antigen binding fragment thereof, are provided. In some embodiments, pharmaceutical packages comprising unit dose forms of an SDC1-specific radiolabeled antibody, or of molecules comprising the SDC1-specific radiolabeled antibody, are provided.

In some instances, the SDC1 antibody or antigen-binding fragment may be an isolated SDC1 antibody or antigen-binding fragment thereof as described in this disclosure. The term “isolated,” as used with reference to a protein (or nucleic acid), denotes that the protein (or nucleic acid) is essentially free of other cellular components with which it is associated in the natural state. It is preferably in a homogeneous state. Purity and homogeneity are typically determined using analytical chemistry techniques such as electrophoresis (e.g., polyacrylamide gel electrophoresis) or chromatography (e.g., high performance liquid chromatography). In some embodiments, an isolated protein (or nucleic acid) is at least 85% pure, at least 90% pure, at least 95% pure, or at least 99% pure.

In some instances, the SDC1 antibody or antigen-binding fragment thereof may be a formulated into virus-like particles (VLPs). VLPs comprise viral protein(s) derived from the structural proteins of a virus. Methods for making and using virus like particles are described in, for example, Garcea and Gissmann, 2004, Current Opinion in Biotechnology 15:513-7.

In some instances, the SDC1 antibody or antigen-binding fragment thereof may be a formulated into subviral dense bodies (DBs). DBs transport proteins into target cells by membrane fusion. Methods for making and using DBs are described in, for example, Pepperl-Klindworth et al., 2003, Gene Therapy 10:278-84.

### VIII. Kits and Packaging

The SDC1 antibodies and antigen binding fragments thereof, or molecules or cells comprising the SDC1-specific antibody or antigen binding fragment thereof, disclosed herein may be used for the preparation of a kit (e.g., a diagnostic test kit or kit for the treatment of a patient). In some embodiments, kits are provided for carrying out any of the methods described herein. The kits of this disclosure may comprise a carrier container being compartmentalized to receive in close confinement one or more containers such as vials, tubes, and the like, each of the containers comprising one of the separate elements to be used in the method. In some embodiments, the SDC1-specific radiolabeled antibody disclosed herein may be used for the preparation of a kit (e.g., an imaging test kit or kit for the treatment of a patient).

In some embodiments, one of the containers may comprise an SDC1 antibody or antigen binding fragment thereof as described in this disclosure that is, or can be, detectably labeled. The kit may also have containers containing buffer(s) and/or a container comprising a reporter-means, such as a biotin-binding protein, such as avidin or streptavidin, bound to a reporter molecule, such as an enzymatic or fluorescent label. For example, a kit for imaging a tumor in a subject with an SDC1 expressing cancer is provided herein. In some embodiments, the kit comprises a container containing a labeled SDC1 antibody or antigen binding fragment thereof. In some embodiments, the kit comprises separate containers containing an SDC1 antibody or antigen binding fragment thereof and a detectable label.

In some embodiments, one of the containers may comprise an SDC1-specific radiolabeled antibody as described in this disclosure for imaging a tumor, and one of the containers may comprise an SDC1-specific radiolabeled antibody as described in this disclosure for treatment of a tumor. In some embodiments, one SDC antibody may be labeled with [⁸⁹Zr], and one SDC antibody may be labeled with [¹⁶¹Tb]. In some embodiments, the kit comprises a container comprising a second therapeutic agent. In some embodiments, the kit may be tailored to specific patients.

An SDC1 antibody or antigen binding fragment thereof, or molecule or cell comprising the SDC1-specific antibody or antigen binding fragment thereof, as described in this disclosure for use in treating cancer patients may be delivered in a pharmaceutical package or kit to doctors, healthcare providers, treatment facilities, or cancer patients. Such packaging is intended to improve patient convenience and compliance with the treatment plan. Typically, the packaging comprises paper (cardboard) or plastic. Also provided herein are SDC1-specific radiolabeled antibody, or molecule or cell comprising the SDC1-specific radiolabeled antibody, as described in this disclosure for use in treating cancer patients may be delivered in a pharmaceutical package or kit to doctors, healthcare providers, or treatment facilities. In some embodiments, the kit or pharmaceutical package further comprises instructions for use (e.g., for administering according to a method as described herein).

In some embodiments, a pharmaceutical package or kit comprises unit dose forms of an SDC1 antibody or antigen binding fragment or molecule or cell comprising the SDC1-specific antibody or antigen binding fragment thereof. In some embodiments, the pharmaceutical package or kit further comprises unit dose forms of one or more of a chemotherapeutic agent, a cytotoxic agent, a radiotherapeutic agent, or an immunotherapeutic agent.

In one embodiment, the kit or pharmaceutical package comprises an SDC1 antibody or antigen binding fragment, or a molecule or cell comprising the SDC1-specific antibody or antigen binding fragment thereof, in a defined, therapeutically effective dose in a single unit dosage form or as separate unit doses. The dose and form of the unit dose (e.g., pre-filled syringe, tablet, capsule, immediate release, delayed release, etc.) can be any doses or forms as described herein.

In one embodiment, the kit or pharmaceutical package includes doses suitable for multiple days of administration, such as one week, one month, or three months.

In certain embodiments, kits are provided for producing a single-dose administration unit. In certain embodiments, kits containing single or multi-chambered pre-filled syringes are included. In certain embodiments, kits containing one or more containers of a formulation described in this disclosure are included.

### IX. Methods of Use

### A. Methods of Detecting SDC1

Methods for detecting the presence of SDC1 expressing cells in a biological sample are provided. In some embodiments, the methods include: (a) contacting said sample with a composition comprising an isolated SDC1 antibody or antigen binding portion thereof as described in this disclosure; and (b) detecting an amount of binding of the isolated antibody or antigen binding portion thereof as a determination of the presence of SDC1 expressing cells. In some embodiments, the biological sample comprises a tumor sample.

In some embodiments, SDC1 expression in cancer cells can be examined by using one or more routine biochemical analyses. In some embodiments, SDC1 expression is determined by detecting protein expression using methods such as Western blot analysis, flow cytometry, and immunohistochemistry staining using an SDC1 antibody or antigen binding portion thereof as described in this disclosure. In some instances, a combination of these methods may be used, or additional methods may also be used such as microarray analysis and RT-PCR.

In some instances, a threshold amount of SDC1 protein expression is used to characterize SDC1 expression as either high or low. A high level of SDC1 protein expression refers to a measure of SDC1 protein expression above a particular threshold. For example, the threshold may be a normal, an average, or a median amount of SDC1 protein expression as measured in a particular set of samples, referred to as a reference population. In some instances, the reference population may be a population of normal/healthy subjects. In other instances, the reference population may be a population of subjects having a particular type of cancer (the same type of cancer that the subject being assessed has). A low level of SDC1 expression refers to the converse of the above. For example, the threshold may be determined by identifying two distinct subgroups in the reference population by dividing samples around a mathematically determined point, such as, without limitation, a median, thus creating a subgroup whose measure is high (i.e., higher than the median) and another subgroup whose measure is low.

Also provided are methods of imaging a tumor in a subject with an SDC1 expressing cancer, the method comprising administering to the subject an isolated antibody or antigen binding portion thereof that is specific for SDC1 that is conjugated to an imaging label and detecting the imaging label in the subject. Imaging methods may be used to assess tumor size and changes in tumor size over or after the course of a treatment administered to the subject. The methods may be useful to assess response of the subject to an administered treatment. In some instances, the methods may be useful to grade the subject's cancer.

### A. Methods of Treatment

Also provided herein are methods to treat, inhibit, or delay progression of a disease or disorder associated with elevated levels of SDC1, such as cancer. In some embodiments, the cancer associated with elevated levels of SDC1 is pancreatic or colorectal cancer. In some other embodiments, the cancer associated with elevated levels of SDC1 is a renal, non-small cell lung, ovarian, bladder, melanoma, prostate, or neuroectodermal cancer, or another cancer disclosed herein. Functioning of SDC1 may be reduced by any suitable therapeutic drug or molecule. Preferably, such substance would be an SDC1 antibody or antigen binding fragment thereof (or a molecule comprising or encoding the SDC1 antibody or antigen binding fragment thereof) as described in this disclosure. The methods comprise administering to a subject a pharmaceutically effective amount of a composition comprising an isolated SDC1-specific antibody or antigen binding portion thereof (or a molecule comprising or encoding the SDC1 antibody or antigen binding fragment thereof) described herein. Also, provided are prognostic and diagnostic methods for cancer based on detection and/or quantitation of SDC1 using an SDC1 antibody or antigen binding fragment as described in this disclosure. Also provided are methods of detecting the presence of SDC1 protein in a sample using the described SDC1 antibodies or antigen binding fragments.

Also provided herein are methods to treat, inhibit, or delay progression of a disease or disorder associated with elevated levels of SDC1, such as cancer. In some embodiments, the cancer associated with elevated levels of SDC1 is pancreatic or colorectal cancer. In some other embodiments, the cancer associated with elevated levels of SDC1 is a renal, non-small cell lung, ovarian, bladder, melanoma, prostate, or neuroectodermal cancer, or another cancer disclosed herein. Functioning of SDC1 may be reduced by an SDC1-specific radiolabeled antibody (or a molecule comprising SDC1-specific radiolabeled antibody described in this disclosure). The methods comprise administering to a subject a pharmaceutically effective amount of a composition comprising an isolated SDC1-specific radiolabeled antibody for imaging as described herein, and treating the subject with a therapeutically effective amount of a composition comprising an isolated SDC1-specific radiolabeled antibody as described herein.

As used throughout, subject can be a vertebrate, more specifically a mammal (e.g., a human, horse, cat, dog, cow, pig, sheep, goat, mouse, rabbit, rat, and guinea pig), birds, reptiles, amphibians, fish, and any other animal. The term does not denote a particular age or sex. Thus, adult and newborn subjects, whether male or female, are intended to be covered. As used herein, “patient” or “subject” may be used interchangeably and includes human and veterinary subjects. The SDC1 antibody or antigen binding portion thereof described herein is useful for treating cancer in humans, including, without limitation, pediatric and geriatric populations, and in animals, e.g., veterinary applications. In one embodiment, the subject is a human.

As used herein the terms “cancer” and “tumor” are used to indicate malignant tissue. The term “cancer” is also used to refer to the disease associated with the presence of malignant tumor cells in an individual, and the term “tumor” is used herein to refer to a plurality of cancer cells that are physically associated with each other. Cancer cells are malignant cells that give rise to cancer, and tumor cells are malignant cells that can form a tumor and thereby give rise to cancer.

The term “cancer,” as used herein, may be used to describe a solid tumor, metastatic cancer, or non-metastatic cancer. In certain embodiments, the cancer may originate in the pancreas, colon, rectum, or lung. In other embodiments, the cancer may originate in the bladder, blood, bone, bone marrow; brain, breast, esophagus, duodenum, small intestine, large intestine, gum, head, kidney, liver, nasopharynx, neck, ovary, pancreas, prostate, skin, stomach, testis, tongue, or uterus.

As used herein, an “effective amount” means the amount of an agent that is effective for producing a desired effect in a subject. The actual dose that comprises the effective amount may depend upon the route of administration, the size and health of the subject, the disorder being treated (e.g., cancer), and the like.

In some embodiments, the SDC1 antibody or antigen binding fragment thereof can directly inhibit growth and induce cell death of cancer cells. In some instances, the SDC1 antibody or antigen binding fragment thereof may inhibit tumor initiation, e.g., by binding to SDC1 expressed by cancer stem cells. In some instances, the SDC1 antibody or antigen binding fragment thereof can sensitize cancer cells to other cancer therapies (e.g., chemotherapy). In some embodiments, the SDC1-specific radiolabeled antibody or antigen binding fragment thereof can directly inhibit growth and induce cell death of cancer cells. In some instances, the SDC1-specific radiolabeled antibody or antigen binding fragment thereof may inhibit tumor initiation, e.g., by binding to SDC1 expressed by cancer stem cells. In some instances, the SDC1-specific radiolabeled antibody or antigen binding fragment thereof can sensitize cancer cells to other cancer therapies (e.g., chemotherapy). In some instances, treating a subject according to the methods described herein inhibits at least one of formation of a tumor, the proliferation of tumor cells, the growth of tumor cells, survival of tumor cells in circulation, or metastasis of tumor cells in the individual. In another embodiment, treating a subject according to the methods described herein may result in tumor growth stasis, reduction of tumor size and, in some instances, elimination of one or more tumors in the subject.

In some embodiments, the SDC1 antibody or antigen binding fragment thereof itself may not be therapeutic but may be used to target a therapeutic agent to cancer stem cells or cancer cells. In such embodiments, the SDC1 antibody or antigen binding fragment thereof need only bind specifically to the SDC1 protein. Thus, in some instances, the SDC1 antibody or antigen binding fragment thereof may be conjugated to a therapeutic pharmaceutical agent.

Also provided are cancer treatment methods using a CAR comprising an SDC1 antibody or antigen binding fragment thereof as described in this disclosure. In some embodiments, these methods comprise using the CAR to redirect the specificity of an immune effector cell (e.g., a T cell) to target a cancer cell (e.g., an SDC1 expressing cancer cell). Thus, provided herein are methods of stimulating an effector cell-mediated response (such as a T cell-mediated immune response) to a target cell population or tissue comprising cancer cells in a mammal, comprising the step of administering to the mammal an effector cell (such as a T cell) that expresses a CAR as described herein. In some embodiments, “stimulating” an immune cell refers to eliciting an effector cell-mediated response (such as a T cell-mediated immune response), which is different from activating an immune cell. CAR-expressing effector cells described herein can be infused to a subject in need of treatment (e.g., a cancer patient). In some embodiments, the infused cell is able to kill (or lead to the killing of) cancer cells in the subject. Formulations and methods for making CAR-expressing effector cells and using them in therapeutic methods are known in the art (see. e.g., Feins et al., 2019, Am. J. Hematol. 94 (S1): S3-S9).

The subject to be treated by any of the methods herein may have one of various of different cancers, including, for example, lymphoma, follicular lymphoma (FL), mantle cell lymphoma (MCL), diffuse large B-cell lymphoma (DLBCL), leukemia, chronic lymphocytic leukemia (CLL), marginal zone lymphoma, myeloma, breast cancer, colon cancer, colorectal cancer, lung cancer, skin cancer, pancreatic cancer, testicular cancer, bladder cancer, cervical cancer, ovarian cancer, uterus cancer, prostate cancer, head and neck, laryngeal cancer, nasopharyngeal cancer, gastric cancer, or adrenal cancer. In certain embodiments, the cancer is pancreatic cancer, colorectal cancer, or lung cancer. In some embodiments, the subject may have a primary cancer. In other embodiments, the subject may have metastatic cancer. In some embodiments, the cancer comprises cells that abnormally express SDC1 at a level above basal expression in corresponding normal/non-cancer cells (i.e., an SDC1 expressing cancer).

In some embodiments of the treatment methods, SDC1 expression (e.g., in cancer cells) can be examined by using one or more routine biochemical analyses before, during, or after treatment. In some embodiments, SDC1 expression is determined by detecting protein expression using methods such as mass spectrometry. Western blot analysis, flow cytometry, positron emission tomography, or immunohistochemistry staining. In some embodiments, such methods comprise use of an SDC1 antibody or antigen binding portion thereof (e.g., as described in this disclosure). In some embodiments, such methods comprise use of an SDC1-specific radiolabeled antibody. In some embodiments, SDC1 expression is determined by detecting mRNA levels using methods such as RT-PCR, RNA sequencing, microarray analysis, and Northern blot analysis. In some embodiments, a combination of these methods may be used, or additional methods known in the art may also be used. In some embodiments, described herein, the SDC1-specific radiolabeled antibody may be used to simultaneously detect the presence of SDC1 and treat tumors presenting SDC1. In some embodiments, the SDC1-specific radiolabeled antibody described herein may not be used to detect if a patient has or is suspected of having cancer. For example, in such embodiments, the patient may already be diagnosed with SDC1+ cancer before the administration of an SDC1-specific radiolabeled antibody as described herein is administered.

“Treat.” “treatment,” and the like are used herein to generally mean obtaining a desired pharmacologic and/or physiologic effect. “Treating” or “treatment” may refer to any indicia of success in the treatment or amelioration of cancer. “Treating” or “treatment” includes the administration of an agent to impede growth of a cancer, to do one or more of the following: cause a cancer to shrink by weight or volume, extend the expected survival time of the subject, or extend the expected time to progression of the tumor, or the like. The effect of treatment can be compared to an individual or pool of individuals not receiving the treatment, or to the same patient prior to treatment or at a different time during treatment.

The term “administer,” as used herein, refers to a method of delivering agents, compounds, or compositions to the desired site of biological action. The pharmaceutical compositions (e.g., as described above) are prepared for administration in a number of ways, including but not limited to injection, ingestion, transfusion, implantation, or transplantation, depending on whether local or systemic treatment is desired, and on the area to be treated. The preparation of such pharmaceutically acceptable compositions is within the ability of one skilled in the art. The compositions are administered via any of several routes of administration, including topical, oral, parenteral, intravenous, intra-articular, intraperitoneal, intramuscular, subcutaneous, intracavity, intralesional, transdermal, intradermal, intrahepatical, intrathecal, intracranial, rectal, transmucosal, intestinal, ocular, otic, nasal, inhalation, or intrabronchial delivery, or any other method known in the art. In some embodiments, the SDC1 antibody or antigen binding fragment thereof is administered intravenously, or through local injection.

In one aspect, provided is a method of treating a subject with cancer, the method comprising administering to the patient a therapeutically effective amount of a composition comprising an SDC1 antibody or antigen binding portion thereof as described in this disclosure. The composition may further comprise a pharmaceutically acceptable carrier.

In some instances, the SDC1 antibody or antigen-binding fragment thereof can be administered via virus-like particles. Virus-like particles may be formulated as described herein and as known in the art.

In some instances, the SDC1 antibody or antigen-binding fragment thereof can be administered by subviral dense bodies. Dense bodies may be formulated as described herein and as known in the art.

In some instances, the SDC1 antibody or antigen-binding fragment thereof can be administered by tegument aggregates. Methods for making and using tegument aggregates are described in International Publication No. WO 2006/110728.

In another aspect, provided is a method of treating a subject with cancer, the method comprising administering to the patient cells that have been genetically engineered, using methods such as those described herein, to express and secrete an SDC1 antibody or antigen binding portion thereof as described herein.

In another aspect, provided is a method of treating a subject with cancer, the method comprising administering to the patient immune cells that express a CAR comprising an SDC1 antibody or antigen binding portion thereof as described herein.

In another aspect, provided is a method of treating a subject with cancer, the method comprising administering to the patient a vector comprising a nucleic acid sequence encoding the SDC1 antibody or antigen binding fragment thereof as described in this disclosure. In some embodiments, the nucleic acid molecule comprises a nucleotide sequence that is at least 90% identical to SEQ ID NO: 9 or SEQ ID NO: 10. In some embodiments, the nucleic acid molecules are at least 90% identical (for example, at least 90%, 91%, 92%, 93%, 94%, 95%, 96%, 97%, 98% or 99% identical) to SEQ ID NO: 9 or SEQ ID NO: 10.

There are a number of compositions and methods which can be used to deliver the nucleic acid molecules and/or polypeptides to cells, either in vitro or in vivo via, for example, expression vectors. These methods and compositions can largely be broken down into two classes: viral based delivery systems and non-viral based delivery systems. Such methods are well known in the art and readily adaptable for use with the compositions and methods described herein.

As used herein, plasmid or viral vectors are agents that transport the disclosed nucleic acids into the cell without undesired degradation and include a promoter yielding expression of the nucleic acid molecule and/or adapter polypeptide in the cells into which it is delivered. Viral vectors are, for example, Adenovirus, Adeno-associated virus, herpes virus, Vaccinia virus, Polio virus, Sindbis, and other RNA viruses, including these viruses with the HIV backbone. Also preferred are any viral families which share the properties of these viruses which make them suitable for use as vectors. Retroviral vectors, in general are described by Coffin et al., 1997, Retroviruses, Cold Spring Harbor Laboratory Press, which is incorporated by reference herein for the vectors and methods of making them. The construction of replication-defective adenoviruses has been described (Berkner et al., 1987, J. Virology 61:1213-20; Massie et al., 1986, Mol. Cell. Biol. 6:2872-83: Haj-Ahmad et al., 1986, J. Virology 57:267-74: Davidson et al., 1987, J. Virology 61:1226-39; Zhang et al., 1993, BioTechniques 15:868-72). The benefit and the use of these viruses as vectors is that they are limited in the extent to which they can spread to other cell types, since they can replicate within an initial infected cell, but are unable to form new infections viral particles. Recombinant adenoviruses have been shown to achieve high efficiency after direct, in vivo delivery to airway epithelium, hepatocytes, vascular endothelium, CNS parenchyma, and a number of other tissue sites. Other useful systems include, for example, replicating and host-restricted non-replicating vaccinia virus vectors. In some instances, the nucleic acid molecules encoding the SDC1 antibodies or antigen-binding fragments thereof can be delivered via virus-like particles.

Non-viral based delivery methods, can include expression vectors comprising nucleic acid molecules and nucleic acid sequences encoding the adapter polypeptides, wherein the nucleic acids are operably linked to an expression control sequence. Suitable vector backbones include, for example, those routinely used in the art such as plasmids, artificial chromosomes, BACs, YACs, or PACs. Numerous vectors and expression systems are commercially available from such corporations as Novagen (Madison, WI), Clonetech (Pal Alto, CA), Stratagene (La Jolla, CA), and Invitrogen/Life Technologies (Carlsbad, CA). Vectors typically contain one or more regulatory regions. Regulatory regions include, without limitation, promoter sequences, enhancer sequences, response elements, protein recognition sites, inducible elements, protein binding sequences, 5′ and 3′ untranslated regions (UTRs), transcriptional start sites, termination sequences, polyadenylation sequences, and introns.

In certain embodiments, the effective amount of a pharmaceutical composition comprising an SDC1-specific antibody or antigen binding fragment thereof to be employed therapeutically depends, for example, upon the therapeutic context and objectives. In certain embodiments, the effective amount of a pharmaceutical composition comprising an SDC1-specific radiolabeled antibody to be employed therapeutically depends, for example, upon the therapeutic context and objectives. One skilled in the art will appreciate that the appropriate dosage levels for treatment, according to certain embodiments, vary depending, in part, upon the molecule delivered, the indication for which an SDC1-specific antibody or antigen binding fragment thereof is being used, the route of administration, and the size (body weight, body surface, or organ size) and/or condition (the age and general health) of the patient. The clinician can titer the dosage and modify the route of administration to obtain the optimal therapeutic effect.

The clinician also selects the frequency of dosing, taking into account the pharmacokinetic parameters of the SDC1-specific antibody or antigen binding fragment thereof (e.g., a SDC1-specific radiolabeled antibody) in the formulation used. Such pharmacokinetic parameters are well known in the art, i.e., the rate of absorption, bioavailability, metabolism, clearance, and the like (see. e.g., Hidalgo-Aragones, 1996, J. Steroid Biochem. Mol. Biol. 58:611-17; Groning, 1996, Pharmazie 51:337-41; Fotherby, 1996, Contraception 54:59-69; Johnson, 1995, J. Pharm. Sci. 84:1144-46; Rohatagi, 1995, Pharmazie 50:610-13: Brophy, 1983, Eur. J. Clin. Pharmacol. 24:103-08: the latest Remington's, supra). In certain embodiments, a clinician administers the composition until a dosage is reached that achieves the desired effect. In certain embodiments, the composition can therefore be administered as a single dose or as two or more doses (which may or may not contain the same amount of the desired molecule) over time, or as a continuous infusion via, for example, an implantation device or catheter. Further refinement of the appropriate dosage is routinely made by those of ordinary skill in the art and is within the ambit of tasks routinely performed by them. In certain embodiments, appropriate dosages can be ascertained through use of appropriate dose-response data.

In certain embodiments, the route of administration of the pharmaceutical composition is in accord with known methods, e.g., orally, through injection by intravenous, intraperitoneal, intracerebral (intra-parenchymal), intracerebral, intraventricular, intramuscular, subcutaneously, intra-ocular, intraarterial, intraportal, or intralesional routes: by sustained release systems or by implantation devices. In certain embodiments, the compositions can be administered by bolus injection or continuously by infusion, or by implantation device. In certain embodiments, individual elements of a combination therapy may be administered by different routes.

In certain embodiments, the composition can be administered locally, e.g., during surgery or topically. Optionally local administration is via implantation of a membrane, sponge, or another appropriate material onto which the desired molecule has been absorbed or encapsulated. In certain embodiments, where an implantation device is used, the device can be implanted into any suitable tissue or organ, and delivery of the desired molecule can be via diffusion, timed-release bolus, or continuous administration.

In certain embodiments, it can be desirable to use a pharmaceutical composition comprising an SDC1 antibody or antigen binding fragment thereof (e.g., an SDC1-specific radiolabeled antibody) in an ex vivo manner. In such instances, cells that have been removed from a subject may be exposed to a pharmaceutical composition comprising an SDC1 antibody or antigen binding fragment thereof after which the cells are subsequently implanted back into the subject.

In some instances, the provided methods may include administering to the subject an SDC1-specific antibody or antigen binding fragment thereof that is conjugated to a therapeutic agent. The therapeutic agent may be at least one of a cytotoxic agent, a chemotherapeutic agent, or an immunosuppressive agent. In some instances, the provided methods may include administering to the subject an SDC1-specific radiolabeled antibody that is co-administered with another therapeutic agent. The therapeutic agent may be at least one of a cytotoxic agent, a chemotherapeutic agent, or an immunosuppressive agent.

In some instances, the provided methods may include administering an SDC1-specific antibody or antigen binding fragment thereof (e.g., an SDC1-specific radiolabeled antibody) and a second form of cancer therapy to the subject. The second form of cancer therapy may include a cytotoxic agent, a chemotherapeutic agent, an immunosuppressive agent (including immune checkpoint inhibitors), or radiation therapy. In some embodiments, the second form of cancer therapy is an antibody (e.g., a monoclonal antibody). For example, in some embodiments, monoclonal antibodies or small molecule inhibitor which may be administered as a second form of cancer therapy include, but are not limited to, a PD1 antibody, a 4-1BB antibody, panitumumab, bevacizumab, cetuximab, adagrasib (MRTX849), or edrecolomab (e.g., for treatment of colorectal cancer): sotorasib (AMG 510), MRTX1133, adagrasib, sintilimab, necitumumab, or nivolumab (e.g., for treatment of non-small cell lung cancer): rituximab (e.g., for treatment of B-cell lymphomas), trastuzumab (e.g., for treatment of breast cancer), and cetuximab (e.g., for treatment of lung cancer).

The methods and compositions, including combination therapies, enhance the therapeutic or protective effect, and/or increase the therapeutic effect of another anti-cancer or anti-hyperproliferative therapy. Therapeutic and prophylactic methods and compositions can be provided in a combined amount effective to achieve the desired effect, such as the killing of a cancer cell and/or the inhibition of cellular hyperproliferation. This process may involve contacting the cells with both an antibody or antibody fragment and a second therapy. A tissue, tumor, or cell can be contacted with one or more compositions or pharmacological formulation(s) comprising one or more of the agents (i.e., antibody or antibody fragment or an anti-cancer agent), or by contacting the tissue, tumor, and/or cell with two or more distinct compositions or formulations, wherein one composition provides 1) an antibody or antibody fragment, 2) an anti-cancer agent, or 3) both an antibody or antibody fragment and an anti-cancer agent. Also, it is contemplated that such a combination therapy can be used in conjunction with chemotherapy, radiotherapy, surgical therapy, immunotherapy, or radioimmunotherapy.

The terms “contacted” and “exposed.” when applied to a cell, are used herein to describe the process by which a therapeutic construct and a chemotherapeutic or radiotherapeutic agent are delivered to a target cell or are placed in direct juxtaposition with the target cell. To achieve cell killing, for example, both agents are delivered to a cell in a combined amount effective to kill the cell or prevent it from dividing.

An antibody or radiolabeled antibody may be administered before, during, after, or in various combinations relative to another anti-cancer treatment. The administrations may be in intervals ranging from concurrently to minutes to days to weeks. In embodiments where the antibody or antibody fragment is provided to a patient separately from another anti-cancer agent, one would generally ensure that a significant period of time did not expire between the time of each delivery, such that the two compounds would still be able to exert an advantageously combined effect on the patient. In such embodiments, it is contemplated that one may provide a patient with the antibody therapy and the anti-cancer therapy within about 6 to 72 hours, about 6 to 48 hours, or about 6 to 24 hours of each other and, more particularly, within about 6-12 hours of each other. In some situations, it may be desirable to extend the time period for treatment significantly where several days (2, 3, 4, 5, 6, or 7) to several weeks (1, 2, 3, 4, 5, 6, 7, or 8) lapse between respective administrations.

In certain embodiments, a course of treatment will last 1-90 days or more (including intervening days). It is contemplated that one agent may be given on any day of day 1 to day 90 (including intervening days) or any combination thereof, and another agent is given on any day of day 1 to day 90) (including intervening days) or any combination thereof. Within a single day (24-hour period), the patient may be given one or multiple administrations of the agent(s). Moreover, after a course of treatment, it is contemplated that there is a period of time at which no anti-cancer treatment is administered. This time period may last 1-7 days, and/or 1-5 weeks, and/or 1-12 months or more, or any time period within these ranges (including intervening days), depending on the condition of the patient, such as their prognosis, strength, health, etc. It is expected that the treatment cycles would be repeated as necessary.

In some embodiments, the SDC1 antibody or antigen binding fragment thereof can be labeled, conjugated, or fused with a therapeutic agent or diagnostic agent (such as an imaging agent). The linkage can be covalent or noncovalent (e.g., ionic). Such antibodies and antibody fragments are referred to antibody-drug conjugates (ADC) or immunoconjugates. The antibody conjugates are useful for the local delivery of therapeutic agents, particularly cytotoxic or cytostatic agents, i.e., drugs to kill or inhibit tumor cells in the treatment of cancer allows targeted delivery of the drug moiety to tumors, and intracellular accumulation therein, where systemic administration of these unconjugated drug agents may result in unacceptable levels of toxicity to normal cells as well as the tumor cells sought to be eliminated. Therapeutic agents include but are not limited to toxins, including but not limited to plant and bacterial toxins, small molecules, peptides, polypeptides, and proteins. Genetically engineered fusion proteins, in which genes encoding for an antibody, or fragments thereof including the Fv region, or peptides can be fused to the genes encoding a toxin to deliver a toxin to the target cell are also provided. As used herein, a target cell or target cells are SDC1 positive cells.

In some embodiments, the SDC1 antibody or antigen binding fragment thereof is conjugated to a moiety that specifically binds to an immune cell. In some embodiments, provided is a bispecific antibody comprising an SDC1 antibody or antigen binding fragment thereof as described herein and an antibody or antigen binding fragment thereof that specifically binds to an immune cell. In some embodiments, the bispecific antibody comprises an SDC1-specific antibody or antigen-binding portion thereof and an antibody moiety that specifically binds to T cells. Such a molecule is referred to as a bispecific T cell engager and may induce T cell-mediated cytotoxicity of SDC1 expressing cancer cells (see. e.g., Zhou et al., 2021, Biomarker Research 9:38). In some embodiments, the bispecific antibody comprises an SDC1-specific antibody or antigen-binding portion thereof and an antibody moiety that specifically binds to natural killer cells (NK cells). Such a molecule is referred to as a NK cell engager and may induce NK cell-mediated cytotoxicity of SDC1 expressing cancer cells (see. e.g., Demaria et al., 2021, European Journal of Immunology 51 (8): 1934-1942). In some embodiments, the isolated antibody is a bispecific antibody that specifically binds SDC1 and PD1 or that specifically binds SDC1 and 4-1BB.

In some embodiments, the therapeutic SDC1-specific radiolabeled antibody may be administered with a second therapeutic radiolabeled SDC1 antibody. In some embodiments, the second therapeutic radiolabeled SDC1 antibody comprises a radiolabel selected from a group consisting of ¹⁶¹Tb, ²²⁵Ac ²⁷⁷Ac, ²¹¹At, ¹²⁸Ba, ¹³¹Ba, ⁷Be, ²⁰⁴Bi, ²⁰⁵Bi, ²⁰⁶Bi, ⁷⁶Br, ⁷⁷Br, ⁸²Br, ¹⁰⁹Cd, ⁴⁷Ca, ¹¹C, ¹⁴C, ³⁶Cl, ⁴⁸Cr, ⁵¹Cr, ⁶²Cu, ⁶⁴Cu, ⁶⁷Cu, ¹⁶⁵Dy, ¹⁵⁵Eu, ¹⁸F, ¹⁵³Gd, ⁶⁶Ga, ⁶⁷Ga, ⁶⁸Ga, ⁷²Ga, ¹⁹⁸Au, ³H ¹⁶⁶Ho, ¹¹¹In, ^{113m} In, ^{115m} In, ¹²³I, ¹²⁵I, ¹³¹I, ¹⁸⁹Ir, ^{191m}Ir, ¹⁹²Ir, ¹⁹⁴Ir, ⁵²Fe, ⁵⁵Fe, ⁵⁹Fe, ¹⁷⁷Lu, ¹⁵O, ^{191m-191}Os, ¹⁰⁹Pd, ³²P, ³³P, ⁴²K, ²²⁶Ra, ¹⁸⁶Re, ¹⁸⁸Re, ^{82m}Rb, ¹⁵³Sm, ⁴⁶Sc, ⁴⁷Sc, ⁷²Se, ⁷⁵Se, ¹⁰⁵Ag, ²²Na, ²⁴Na, ⁸⁹Sr, ³⁵S, ³⁸S, ¹⁷⁷Ta, ⁹⁶Tc, ^{99m}Tc, ²⁰¹Tl, ²⁰²Tl, ¹¹³Sn, ^{117m}Sn, ¹²¹Sn, ¹⁶⁶Yb, ¹⁶⁹Yb, ¹⁷⁵Yb, ⁸⁸Y, ⁹⁰Y, ⁶²Zn, and ⁶⁵Zn. In some embodiments, the radiolabeled antibody as described herein may generate a dual emission of medium energy beta particles and Auger electrons that may interact favorably with the second theranostic that may emit alpha particles. In some embodiments, the methods described herein may include administration of an SDC1-specific [161Tb]-radiolabeled antibody in combination with an SDC1-specific [225 Ac]-radiolabeled antibody. In some embodiments, the selected combination as described herein may result in a reduced toxicity as compared to a single SDC1-specific radiolabeled antibody.

Other examples of therapeutic agents include chemotherapeutic agents, a radiotherapeutic agent, and immunotherapeutic agent, as well as combinations thereof. In this way, the antibody or peptide complex delivered to the subject can be multifunctional, in that it exerts one therapeutic effect by binding to the SDC1 protein and a second therapeutic effect by delivering a supplemental therapeutic agent. In some embodiments, the treatment methods provided herein may further comprise administering an additional therapeutic agent. Some examples of additional therapeutic agents include chemotherapeutic agents, a radiotherapeutic agent, and immunotherapeutic agent, as well as combinations thereof.

The therapeutic agent can act extracellularly, for example by initiating or affecting an immune response, or it can act intracellularly, either directly by translocating through the cell membrane or indirectly by, for example, affecting transmembrane cell signaling. The therapeutic agent is optionally cleavable from the SDC1 antibody or antigen binding fragment thereof. Cleavage can be autolytic, accomplished by proteolysis, or affected by contacting the cell with a cleavage agent.

In some embodiments, the therapeutic agent is a cytotoxic agent. A cytotoxin or cytotoxic agent includes any agent that is detrimental to cells. Examples of toxins or toxin moieties include diphtheria, ricin, streptavidin, and modifications thereof. Additional examples include paclitaxel, cisplatin, carboplatin, cytochalasin B, gramicidin D, ethidium bromide, emetine, etoposide, tenoposide, colchicin, dihydroxy anthracin dione, mitoxantrone, mithramycin, actinomycin D, 1-dehydrotestosterone, glucocorticoids, procaine, tetracaine, lidocaine, propranolol, puromycin and analogs or homologs thereof. Therapeutic agents include, but are not limited to, antimetabolites (e.g., methotrexate, 6-mercaptopurine, 6-thioguanine, cytarabine, 5-fluorouracil, decarbazine), alkylating agents (e.g., mechlorethamine, thiotepa, chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine). Cytotoxic peptides such as auristatin (antineoplastic) peptides auristatin E (AE) and monomethylauristatin (MMAE), which are synthetic analogs of dolastatin, may also be conjugated to the SDC1-specific antibody or antigen binding fragment thereof. In some embodiments, the SDC1-specific antibody or antigen binding fragment thereof may be conjugated to a radioactive metal ion.

As referred to herein, a chemotherapeutic agent is a chemical compound useful in the treatment of cancer. Examples of chemotherapeutic agents include erlotinib (such as TARCEVA®, Genentech/OSI Pharm.), bortezomib (such as VELCADE®, Millenium Pharm.), fulvestrant (such as FASLODEX®, AstraZeneca), sutent (such as SU11248. Pfizer), letrozole (such as FEMARA®, Novartis), imatinib mesylate (such as GLEEVEC®, Novartis), PTK787/ZK222584 (Novartis), oxaliplatin (such as Eloxatin®, Sanofi), 5-fluorouracil (5-FU), leucovorin, rapamycin (also known as sirolimus) (such as RAPAMUNE®, Wyeth), lapatinib (such as TYKERB®, GSK572016. GlaxoSmithKline), lonafarnib (such as SCH 66336), sorafenib (such as BAY43-9006. Bayer Labs.), capecitabine (such as XELODA®, Roche), docetaxel (such as TAXOTERE®), and gefitinib (such as IRESSA®, Astrazeneca), AG1478, AG1571 (such as SU 5271; Sugen Inc.), alkylating agents such as thiotepa and cyclosphosphamide (such as CYTOXAN®); alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues. KW-2189 and CB1-TMI); eleutherobin; pancratistatin; a sarcodictyin; spongistatin; nitrogen mustards such as chlorambucil, chlomaphazine, cholophosphamide, estramustine, ifosfamide, mechlorethamine, mechlorethamine oxide hydrochloride, melphalan, novembichin, phenesterine, prednimustine, trofosfamide, uracil mustard: nitrosoureas such as carmustine, chlorozotocin, fotemustine, lomustine, nimustine, and ranimnustine: antibiotics such as the enediyne antibiotics (e.g., calicheamicin, particularly calicheamicin γ₁^{I} and calicheamicin θ₁^{I}); dynemicin, including dynemicin A; bisphosphonates, such as clodronate; an esperamicin; as well as neocarzinostatin chromophore and related chromoprotein enedivne antibiotic chromophores), aclacinomysins, actinomycin, anthramycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (such as ADRIAMYCIN®, including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfornithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; Trametes Versicolor polysaccharide-K (Krestin, PSK) (JHS Natural Products, Eugene, OR); razoxane; rhizoxin; sizofiran; spirogermanium; tenuazonic acid; triaziquone; 2,2′, 2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; cytarabine (cytosine arabinoside, “Ara-C”); cyclophosphamide; thiotepa; taxoids, e.g., paclitaxel (such as TAXOL R, Bristol-Myers Squibb Oncology. Princeton. N.J.), ABRAXANE™ (a Cremophor-free, albumin-engineered nanoparticle formulation of paclitaxel (American Pharmaceutical Partners. Schaumberg. IL)), and doxetaxel (such as TAXOTERE®, Rhône-Poulenc Rorer. Antony. France); chloranbucil; gemcitabine (such as GEMZAR®): 6-thioguanine; mercaptopurine; methotrexate; platinum analogs such as cisplatin and carboplatin; vinblastine; platinum; etoposide (VP-16); ifosfamide; mitoxantrone; vincristine; vinorelbine (such as NAVELBINE®); novantrone; teniposide: edatrexate: daunomycin: aminopterin; xeloda: ibandronate: CPT-11: topoisomerase inhibitor RFS 2000; difluorometlhylomithine (DMFO): retinoids such as retinoic acid; capecitabine; and pharmaceutically acceptable salts, acids or derivatives of any of the above.

Chemotherapeutic agents, as used herein, also refers to (i) anti-hormonal agents that act to regulate or inhibit hormone action on tumors such as anti-estrogens and selective estrogen receptor modulators (SERMs), including, for example, tamoxifen (including NOLVADEX® tamoxifen), raloxifene, droloxifene, 4-hydroxytamoxifen, trioxifene, keoxifene, LY 117018, onapristone, and toremifene (such as FARESTONR); (ii) aromatase inhibitors that inhibit the enzyme aromatase, which regulates estrogen production in the adrenal glands, such as, for example, 4 (5)-imidazoles, aminoglutethimide, megestrol acetate (such as MEGASE®), exemestane (such as AROMASIN®), formestanie, fadrozole, vorozole (such as RIVISOR®), letrozole (such as FEMARA®), and anastrozole (such as ARIMIDEX®); (iii) anti-androgens such as flutamide, nilutamide, bicalutamide, leuprolide, and goserelin: as well as troxacitabine (a 1,3-dioxolane nucleoside cytosine analog); (iv) aromatase inhibitors: (v) protein kinase inhibitors: (vi) lipid kinase inhibitors: (vii) antisense oligonucleotides, particularly those which inhibit expression of genes in signaling pathways implicated in aberrant cell proliferation, such as, for example, PKC-alpha. Ralf and H-Ras: (viii) VEGF receptor and angiogenesis inhibitors (including ribozymes such as ANGIOZYME®) and a HER2 expression inhibitor; (ix) vaccines such as gene therapy vaccines, for example, ALLOVECTIN-7® vaccine (plasmid/lipid complex containing the DNA sequences encoding HLA-B7 and ß2 microglobulin). LEUVECTIN® vaccine (plasmid DNA expression vector encoding interleukin-2 (IL-2) complexed with a lipid delivery vehicle (DMRIE/DOPE)), and VAXID® vaccine (patient-specific naked DNA vaccine): IL-2 or aldesleukin (such as PROLEUKIN®): topoisomerase 1 inhibitors (such as TOPOTECAN®); gonadotropin-releasing hormone antagonists (such as ABARELIX®); (x) anti-angiogenic agents such as bevacizumab (such as AVASTIN®, Genentech); and (xi) pharmaceutically acceptable salts, acids or derivatives of any of the above.

In some embodiments, the treatment methods provided herein may further comprise administering an immunosuppressive agent such as an immune checkpoint inhibitor as part of the method. These treatments work by “taking the brakes off” the immune system (are immunosuppressive), allowing it to mount a stronger and more effective attack against cancer, Several different types of checkpoint inhibitors, targeting different checkpoints or “brakes” on immune cells, are currently in use. Immune checkpoint proteins that may be targeted by immune checkpoint blockade include adenosine A2A receptor (A2AR), B7-H3 (also known as CD276), B and T lymphocyte attenuator (BTLA), CCL5, CD27, CD38, CD8A, CMKLR1, cytotoxic T-lymphocyte-associated protein 4 (CTLA-4, also known as CD152), CXCL9, CXCR5, glucocorticoid-induced tumor necrosis factor receptor-related protein (GITR), HLA-DRB 1, ICOS (also known as CD278), HLA-DQAI, HLA-E, indoleamine 2,3-dioxygenase 1 (IDO1), killer-cell immunoglobulin (KIR), lymphocyte activation gene-3 (LAG-3, also known as CD223), Mer tyrosine kinase (MerTK), NKG7, OX40) (also known as CD134), programmed death 1 (PD-1), programmed death-ligand 1 (PD-L1, also known as CD274), PDCD1LG2, PSMB 10, ST A Tl, T cell immunoreceptor with lg and ITIM domains (TI GIT), T-cell immunoglobulin domain and mucin domain 3 (TIM-3), and V-domain lg suppressor of T cell activation (VISTA, also known as C10orf54). In particular, the immune checkpoint inhibitors target the PD-1 axis and/or CTLA-4. Exemplary immunosuppressive agents are PD-1 inhibitors (such as nivolumab and pembrolizumab), PD-L1 inhibitors (such as atezolizumab, durvalumab, and avelumab), and CTLA-4 inhibitors (such as ipilimumab). In one example, the second form of cancer therapy comprises a PD-L1 inhibitor, a PD-1 inhibitor, or a CTLA4 inhibitor. In some instances, combinations of such inhibitors can be administered. In some instances, the PD-L1 inhibitor, the PD-1 inhibitor, and/or the CTLA4 inhibitor may be an inhibitory antibody that binds specifically to PD-L1, PD-1, or CTLA4, respectively.

In some instances, the treatment methods provided herein may further comprise administering radiation therapy to the subject. Radiation therapy uses high-energy radiation to shrink tumors and kill cancer cells. X-rays, gamma rays, and charged particles are types of radiation used for cancer treatment. The radiation may be delivered by a machine outside the body (external-beam radiation therapy), or it may come from radioactive material placed in the body near cancer cells (internal radiation therapy, also called brachytherapy). Systemic radiation therapy uses radioactive substances, such as radioactive iodine, that travel in the blood to kill cancer cells.

### B. Diagnostic Methods

In another aspect, provided are methods of assessing eligibility of a subject for inclusion in or exclusion from a clinical trial of or treatment with an SDC1 targeted therapy using an SDC1 antibody or antigen binding fragment thereof. The method comprises (a) measuring in a tumor sample from a subject the amount of SDC1; (b) determining if the subject has a cancer characterized as having a high level of SDC1 expression; and (c) indicating that the subject is eligible for a clinical trial of or treatment with an SDC1 targeted therapy if the subject's cancer is characterized as having a high level of SDC1 expression, i.e., above a predetermined threshold or that the subject is ineligible for a clinical trial of treatment with the SDC1 targeted therapy if the subject's cancer is characterized as having a low level of SDC1 expression, i.e., below a predetermined threshold. In some instances, the threshold level is a median amount of SDC1 determined in a reference population of patients having the same kind of cancer as the subject. In another instance, the threshold level is an optimal amount of SDC1 determined in a reference population of patients having the same kind of cancer as the subject. “Optimal cutoff” as used herein, refers to the value of a predetermined measure on subjects exhibiting certain attributes that allow the best discrimination between two categories of an attribute. For example, finding a value for an optimal cutoff that allows one to best discriminate between two categories (subgroups) of patients for determining at least one of overall survival, time to disease progression, progression-free survival, and likelihood to respond to treatment (e.g., based on clinical assessment using the RECIST criteria, e.g., Eisenhauer, E. A., et al., 2009, Eur. J. Cancer 45:228-247, or the like as recognized in the medical field). Optimal cutoffs are used to separate the subjects with values lower than or higher than the optimal cutoff to optimize the prediction model, for example, without limitation, to maximize the specificity of the model, maximize the sensitivity of the model, maximize the difference in outcome, or minimize the p-value from hazard ratio or a difference in response.

In another aspect, provided are methods for assessing responsiveness of a subject with cancer to an SDC1 antibody or antigen binding fragment thereof comprising: (a) measuring in a tumor sample from a subject the amount of SDC1; (c) determining if the subject has a cancer characterized as having a high level of SDC1 expression; and (d) indicating that the subject is more likely to respond to the SDC1 antibody or antigen binding fragment thereof if the subject's cancer is characterized as having a high level of SDC1 expression. Conversely, if the subject's cancer is characterized as having a low level of SDC1 expression, the subject is less likely to respond to an SDC1 antibody or antigen binding fragment thereof. In some instances, the amount of SDC1 in the tumor sample is measured using an SDC1 antibody or antigen binding fragment thereof as described herein.

In another aspect, provided are methods to diagnose cancer in a subject. Specifically, the diagnosis may be of an SDC1 expressing cancer. The method may comprise measuring in a sample from a subject the amount of SDC1 and diagnosing the subject with cancer if the amount of SDC1 expression in the sample is high. In some instances, the method may comprise (a) measuring in a tumor sample from a subject the amount of SDC1 using an SDC1 antibody or antigen binding fragment thereof; and (c) determining if the subject has a cancer characterized as having a high level of SDC1 expression. Conversely, if the amount of SDC1 expression in the sample or the subject's cancer low level, the subject may not be diagnosed with cancer or may not be diagnosed with an SDC1 expressing cancer.

In some instances, to diagnose cancer in a subject, or to characterize a subject's cancer, a biopsy is typically taken from a subject having an abnormal tissue growth, such as a tumor. Samples may be formalin-fixed, paraffin-embedded tissue samples obtained from the subject's cancer (tumor). In other instances, such as where circulating tumor cells are to be assessed, the sample from the subject is a blood, plasma, or lymph sample. Typically, the tissue or cells of the patient sample reexamined under a microscope in order to confirm the diagnosis and/or assess information about the tumor. In some cases, additional tests may need to be performed on the proteins, DNA, and/or mRNA of the cells in the ample to verify the diagnosis or characterization.

Also provided are methods of monitoring response of a subject with an SDC1 expressing cancer to cancer therapy. The methods include administering to the subject an SDC1-specific antibody or antigen-binding fragment thereof conjugated to an imaging label at a first time point prior to the subject before the subject receives cancer therapy, detecting the imaging label in the subject to obtain a first image of the tumor, administering to the subject an SDC1-specific antibody or antigen-binding fragment thereof conjugated to an imaging label at a second time point after the subject receives cancer therapy, detecting the imaging label in the subject to obtain a second image of the tumor; and comparing the first image to the second image to determine whether a change in tumor size has occurred. In some instances, the steps of administering to the subject an SDC1-specific antibody or antigen-binding fragment thereof conjugated to an imaging label at a first time point after the subject receives cancer therapy, detecting the imaging label in the subject to obtain a second image of the tumor; and comparing the first image to the second image to determine whether a change in tumor size has occurred may be repeated at a third time point (or additional time points) after the subject receives cancer therapy.

Also provided are methods of monitoring response of a subject with an SDC1 expressing cancer to cancer therapy. The methods include detecting the presence of a tumor in the subject at a first time point before the subject receives cancer therapy by administering a first radiolabeled antibody comprising a syndecan 1 (SDC1) antibody or antibody fragment thereof conjugated to a first chelating agent and a first radioactive moiety that is an imaging agent and obtaining a first image of the tumor: treating the subject by administering a therapeutically effective amount of a second radiolabeled antibody comprising the SDC1 antibody or antibody fragment thereof conjugated to a second chelating agent and a second radioactive moiety that is a therapeutic agent: detecting the presence or absence of the tumor in the subject at a second time point after the subject has been treated with cancer therapy by administering the first radiolabeled antibody and obtaining a second image of the tumor; and comparing the first image to the second image to determine whether a change in tumor size has occurred.

In one embodiment, a subject is administered a labeled SDC1 antibody or antigen binding fragment thereof as described in this disclosure that is conjugated to an imaging agent. The labeled SDC1 antibody or antigen binding fragment thereof is allowed to incubate in vivo and bind to SDC1 in the subject's tissues. The imaging label is thereby localized to tumor cells or tissues, and the localized imaging label is detected using an appropriate imaging device as known to those skilled in the art.

The imaging agent may carry a bioluminescent or chemiluminescent label. Such labels include polypeptides known to be fluorescent, bioluminescent or chemiluminescent, or that act as enzymes on a specific substrate (reagent), or can generate a fluorescent, bioluminescent or chemiluminescent molecule. Examples of bioluminescent or chemiluminescent labels include luciferases, aequorin, obelin, mnemiopsin, berovin, a phenanthridinium ester, and variations thereof and combinations thereof. A substrate for the bioluminescent or chemiluminescent polypeptide may also be used in imaging. For example, the chemiluminescent polypeptide can be luciferase and the reagent luciferin. A substrate for a bioluminescent or chemiluminescent label can be administered before, at the same time (e.g., in the same formulation), or after administration of the agent.

In some instances, the steps of detecting the presence or absence of the tumor in the subject at a time point after the subject has been treated with cancer therapy by administering the first radiolabeled antibody and obtaining another image of the tumor; and comparing the images from before and after treatment to determine whether a change in tumor size has occurred are repeated (one or more times) to determine whether a change in tumor size has occurred after the subject receives additional cancer therapy. In some embodiments, the cancer therapy administered may be the radiolabeled antibody alone or in combination with a second therapeutic agent as described above. In embodiments where the cancer therapy is the radiolabeled antibody alone, the method of monitoring may run simultaneously to the method of treating a patient having SDC1+ cancer.

In some embodiments, the cancer therapy may include additional radiolabeled antibodies that may be administered in combination with the [⁸⁹Zr] or [¹⁶¹Tb]-labeled antibodies. Such radiolabels are disclosed in section III of the detailed description above. In some embodiments, the second anti-cancer therapy may include a chemotherapy, molecular targeted therapy, immunotherapy, radiotherapy, radioimmunotherapy, phototherapy, gene therapy, surgery, hormonal therapy, epigenetic modulation, anti-angiogenic therapy or cytokine therapy.

The SDC1-specific radiolabeled antibody is allowed to incubate in vivo and bind to SDC1 in the subject's tissues. The radiolabel is thereby localized to tumor cells or tissues, and the localized imaging label is detected using an appropriate imaging device as known to those skilled in the art.

The imaging agent may include a paramagnetic compound, such as a polypeptide chelated to a metal (e.g., a metalloporphyrin). The paramagnetic compound may also include a monocrystalline nanoparticle, e.g., a nanoparticle including a lanthanide (e.g., Gd) or iron oxide; or a metal ion such as a lanthanide. Examples of elements that are useful in magnetic resonance imaging include gadolinium, terbium, tin, iron, or isotopes thereof.

Whole body imaging techniques using radioisotope labeled agents can be used for locating diseased cells and tissues (e.g., primary tumors and tumors which have metastasized). In some cases, the labeled agents for locating the tumor tissue or cells are administered intravenously. The bio-distribution of the label can be monitored by scintigraphy, and accumulations of the label are related to the presence of SDC1 or other tumor markers. Whole body imaging techniques are described in, e.g., U.S. Pat. Nos. 4,036,945 and 4,311,688. In embodiments wherein imaging is performed on the patient, the physician may administer a preselected dose that may be based on concentrations known by those skilled in the art. In some embodiments, the SDC1-specific radiolabeled antibody may be administered at a concentration to deliver about 100 μCi to 400 μCi.

An image according to this disclosure can be generated by computer assisted tomography (CAT or CT), magnetic resonance spectroscopy (MRS) image, magnetic resonance imaging (MRI), positron emission tomography (PET), single-photon emission computed tomography (SPECT), or bioluminescence imaging (BLI) or equivalent.

Computer assisted tomography (CAT) and computerized axial tomography (CAT) systems and devices well known in the art can be used to generate an image. (See, for example, U.S. Pat. Nos. 6,151,377; 5,946,371; 5,446,799; 5,406,479; 5,208,581; and 5,109,397.) The imaging methods may also utilize animal imaging modalities, such as MicroCAT™. (ImTek, Inc.).

Magnetic resonance imaging (MRI) systems and devices well known in the art can be used for imaging. For a description of MRI methods and devices, see, for example, U.S. Pat. Nos. 6,151,377. MRI and supporting devices are commercially available, for example, from Bruker Medical GMBH; Caprius; Esaote Biomedica; Fonar; GE Medical Systems (GEMS); Hitachi Medical Systems America: Intermagnetics General Corporation: Lunar Corp.; MagneVu; Marconi Medicals; Philips Medical Systems; Shimadzu; Siemens; Toshiba America Medical Systems; including imaging systems, by, e.g., Silicon Graphics.

Positron emission tomography imaging (PET) systems and devices well known in the art can be used for imaging. For example, an imaging method of this disclosure may use the system designated Pet VI located at Brookhaven National Laboratory. For descriptions of PET systems and devices, see, for example, U.S. Pat. Nos. 6,151,377. Animal imaging modalities such as micro-PETs (Concorde Microsystems, Inc.) can also be used.

Single-photon emission computed tomography (SPECT) systems and devices well known in the art can be used for imaging. (See, for example, U.S. Pat. Nos. 6,115,446; 6,072,177; 5,608,221; 5,600,145; 5,210,421; 5,103,098) Imaging methods may also use animal imaging modalities, such as micro-SPECTs.

Sensitive photon detection systems can be used to detect bioluminescent and fluorescent proteins externally; see for example, Contag, 2000, Neoplasia 2:41-52; and Zhang, 1994, Clin. Exp. Metastasis, 12:87-92. The imaging methods of the disclosure can be practiced using any such photon detection device, for example, an intensified charge-coupled device (ICCD) camera coupled to an image processor. Photo detection devices are also commercially available from Xenogen, Hamamatsue.

Disclosed herein are materials, compositions, and methods that can be used for, can be used in conjunction with or can be used in preparation for the disclosed embodiments. These and other materials are disclosed herein, and it is understood that when combinations, subsets, interactions, groups, etc. of these materials are disclosed that while specific reference of each various individual and collective combinations and permutations of these compositions may not be explicitly disclosed, each is specifically contemplated and described herein. For example, if a method is disclosed and discussed, and a number of modifications that can be made to a number of molecules included in the method are discussed, each and every combination and permutation of the method, and the modifications that are possible are specifically contemplated unless specifically indicated to the contrary. Likewise, any subset or combination of these is also specifically contemplated and disclosed. This concept applies to all aspects of this disclosure including, but not limited to, steps in methods using the disclosed compositions. Thus, if there are various additional steps that can be performed, it is understood that each of these additional steps can be performed with any specific method steps or combination of method steps of the disclosed methods, and that each such combination or subset of combinations is specifically contemplated and should be considered disclosed.

Publications cited herein and the material for which they are cited are hereby specifically incorporated by reference in their entireties. The following description provides further non-limiting examples of the disclosed compositions and methods.

### EXAMPLES

The following examples are offered to illustrate, but not to limit, the claimed invention.

### Example 1. SDC1 is a Potential Biomarker for mKras Cancers

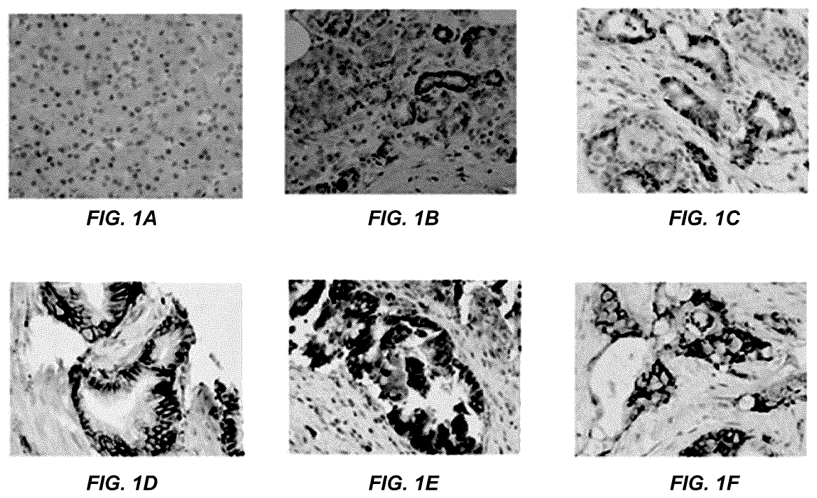

Given the importance of surface proteins as druggable targets and clinical biomarkers, an unbiased, multidimensional target discovery platform to query mKRAS-dependent changes in pancreatic ductal adenocarcinoma cancer (PDAC) cell surface protein composition was previously developed (Yao et al., 2019, Nature 568:410-14). The unbiased study identified SDC1 as the top candidate surfaceome protein among others that are highly expressed upon mKRAS activation in PDAC cells. To assess SDC1's potential use, expression of SDC1 was evaluated using immunohistochemistry in primary human PDAC tissues was performed (FIGS. 1A-1F). IHC staining revealed SDC1 is highly expressed in a majority of premalignant lesions (metaplastic (FIG. 1B) and pancreatic intrapethelial neoplasia (PanIN) lesions (FIG. 1C-FIG. 1E)) as well as in invasive carcinomas (FIG. 1F).

### Example 2. SDC1 Mediates Macropinocytosis to Promote Tumor Growth

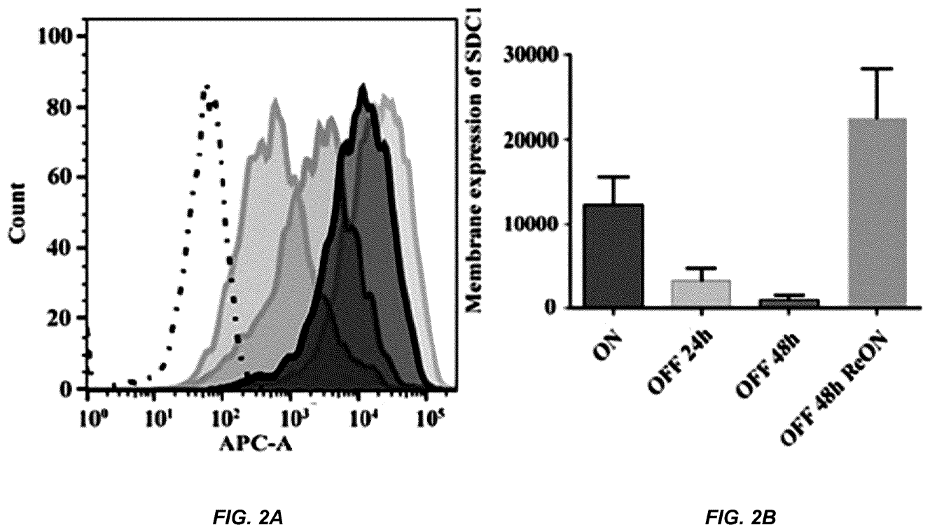

Experiments were conducted to determine the regulation of SDC1 by KRAS signaling in iKras PDAC cells. In one experiment, mouse-derived primary pancreatic cancer cells were initially maintained in Roswell Park Memorial Institute (RPMI)-1640 medium containing 10% Tet-approved Fetal Bovine Serum (FBS) (Clontech), and 1 mg/ml doxycycline (Clontech). Doxycycline was withdrawn and reintroduced to the iKras PDAC cell culture. Evaluation of surface SDC1 expression was evaluated by flow cytometry. Fluorescence activated cell sorting (FACS) demonstrated that upon doxycycline withdrawal (OFF) and reintroduction (Re-ON) in iKras PDAC cells, SDC1 was tightly controlled by mKRAS expression in PDAC cells (FIGS. 2A-2B). FIG. 2A shows the results of the FACS analysis, and FIG. 2B shows the quantitation of that analysis. In brief, SDC1 expression was higher in cells that were treated with doxycycline (before doxycycline withdrawal (far left bar, FIG. 2B) and after reintroduction of doxycycline (far right bar, FIG. 2B) when compared to the cells that incubated in media in which doxycycline was withdrawn for 24-hours or 48-hours (middle two bars, FIG. 2B).

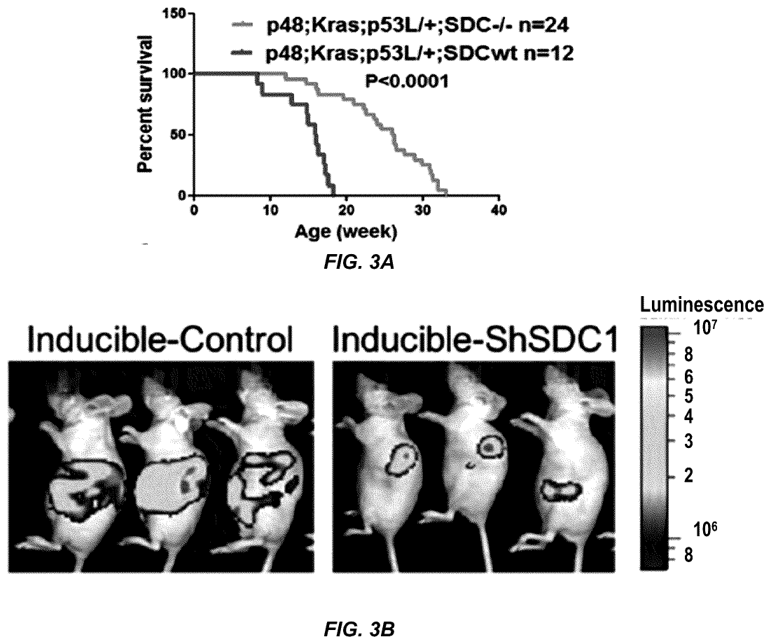

Further analysis of SDC1 role in mKRAS-induced PDAC revealed that SDC1 loss delayed tumor progression and prolonged overall mice survival (FIGS. 3A-3B). For this experiment, mice with the SDC1−/−allele were crossed with p48Cre_LSL_KrasG12D Tp53L/+ mice ((KPC) clinically relevant mouse model for PDAC) to produce SDC1 negative KPC mice. The 50% survival rate of the SDC1 negative KPC mice was increased from under 16.7 weeks (KPC mice) to about 25 weeks (SDC1 negative KPC mice). Additionally, presence of the wild type SDC1 allele resulted in a 0% survival at about 19 weeks while the absence of the wild type SDC1 allele increased survival to over 30 weeks with 0% survival at about 32 weeks (FIG. 3A).

Additional experiments in PDAC patient derived xenograft (PDX) models confirmed the significance of SDC1 on tumor growth. In these experiments, inducible shRNA (short hairpin RNA) was introduced by lentiviral infection to knockdown SDC1 expression in PDX PATC53 cells. The left panel of FIG. 3B shows the size of tumors in control mice, and the right panel of FIG. 3B shows the size of the tumors in mice in which shRNA was used to deplete SDC1. The depletion of SDC1 expression in these experiments significantly inhibited tumor growth those mice (FIG. 3B), further demonstrating the importance of SDC1 in PDAC maintenance.

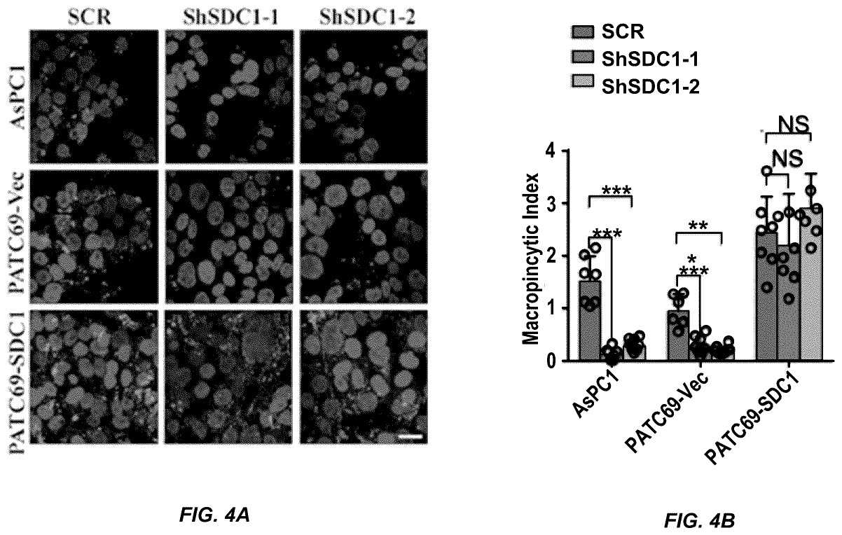

Finally, experiments were conducted to confirm the role of surface expressed SDC1, using human AsPC1 and PDX-derived PATC69 cells (PDAC cell lines). The cells were infected with lentiviral particles to introduce SDC1 shRNA (for SDC1 depletion) or SCR shRNA (scrambled RNA control). Cells were seeded in 8-well chamber slides (LabTek). After cell attachment, cells were serum-starved for 12-18 hours. Macropinosomes were marked utilizing a high molecular weight TMR-dextran (Fina Biosolutions) uptake assay in which TMR-dextran was added to serum-free medium at a final concentration of 1 mg/ml for 35 minutes at 37° C. At the end of the incubation period, cells were rinsed five times in cold phosphate buffer saline (PBS) and immediately fixed in 4% paraformaldehyde. Cells were DAPI-treated to stain nuclei, and coverslips were mounted onto slides using DAKO mounting medium (DAKO). Images were captured using FV1000 Olympus Confocal Microscope system (FIG. 4A) and analyzed using the ‘Analyze Particles’ feature in ImageJ (NIH). The total particle area per cell was determined from at least 6 fields that were randomly selected from different regions across the entirety of each sample (FIG. 4B). Upon SDC1 depletion, macropinocytosis was significantly impaired in AsPC1 and PDX-derived PATC69 cells. Thus, the data indicate SDC1 is a key mKRAS surrogate for PDAC development and maintenance.

### Example 3. SDC1 is Critical for the Acquired Resistance to KRAS Targeted Therapy in PDAC and Colorectal Carcinomas (CRC)

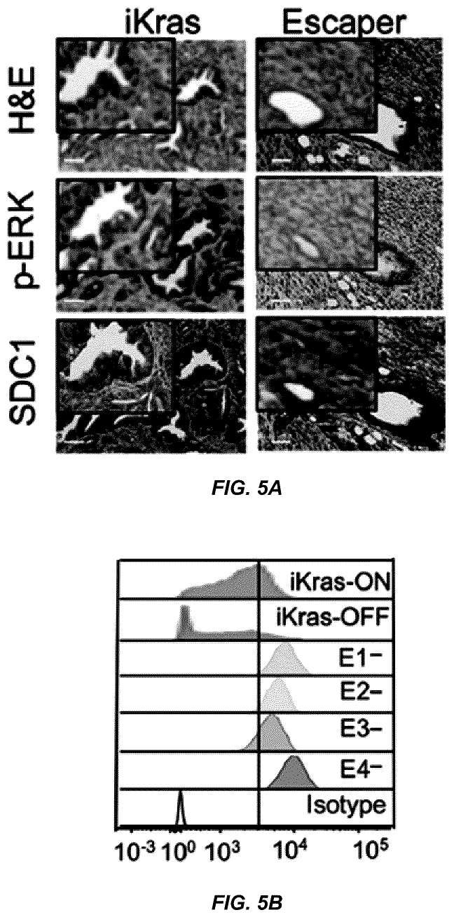

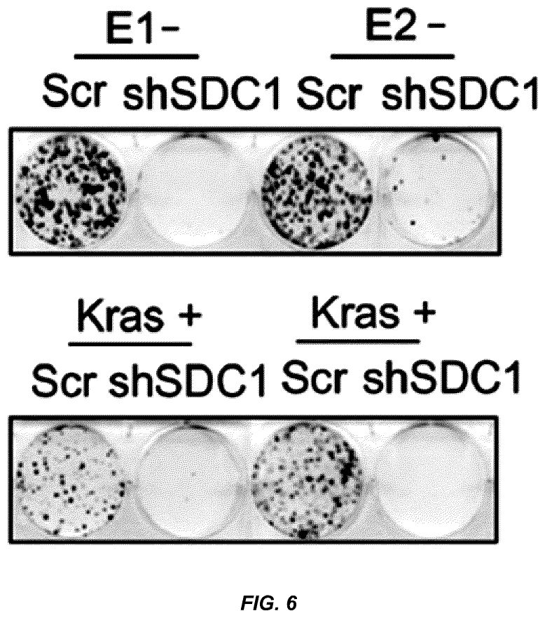

Previously, it was demonstrated that spontaneous tumor relapse following Kras^{G12D} extinction, in doxycycline-inducible KRAD^{G12D}-driven (iKras) GEM models of PDAC, was driven by mRKAS-independent mechanisms (mKRAS-Escapers: E-) or oncogene reactivation (mKRAS-reactivated: E+) (See, e.g., Kapoor et al., 2014, Cell 158:185-97). Interestingly, while Kras^{G12D} extinction resulted in a dramatic downregulation of cell surface SDC1, membrane SDC1 level was comparable between E− and E+ tumors from the iKras model or tumor-derived primary cultures (FIGS. 5A-5B), although E-tumors exhibited much weaker MAPK activity that the E+ tumors (data not shown). These results suggest that the plasma membrane SDC1 was recovered following bypass of mKRAS dependency. To further analyze the iKras cells and iKras-escaper cells, the cells were infected with lentiviral particles to introduce SDC1 shRNA (for SDC1 depletion) or SCR shRNA (scrambled RNA control). Genetic depletion of SDC1 further abolished the clonogenic activity of E-tumor cells (FIG. 6, top panel (2 escaper cells)), as well as E+ cells derived from iKras relapsed tumor cells (FIG. 6, bottom panel), indicating that SDC1 expression is essential for the in vitro growth of both mKRAS-independent and mKRAS-dependent cancer cells. Together, these in vitro and in vivo data demonstrate that SDC1 expression is necessary and sufficient to drive cancer cell growth and to maintain tumorigenic potential in the absence of mKRAS transgene expression.

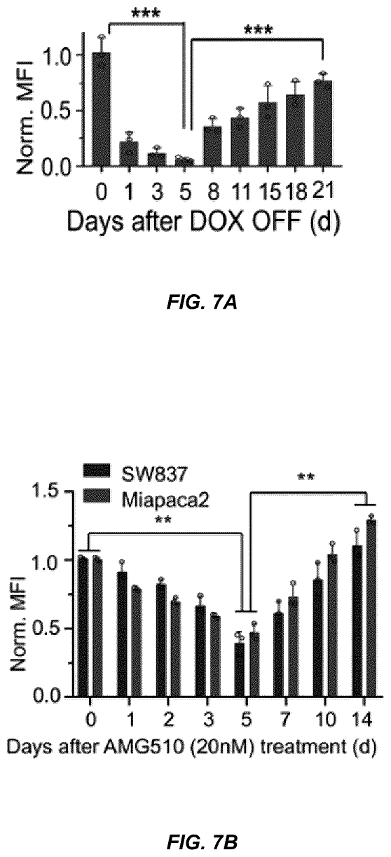

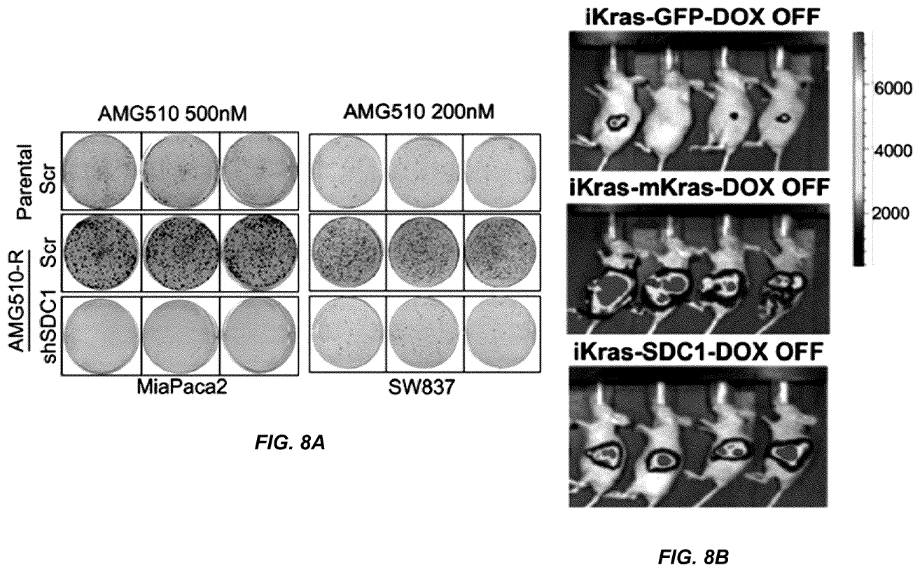

The recovery of surface SDC1 expression levels in the mKRAS-escapers prompted further investigation into SDC1 expression upon pharmacological inhibition of mKRAS or its downstream MAPK pathway. Specifically, the initial depletion and subsequent restoration of surface SDC1 accumulation was observed upon treatment with the KRAS^{G12C} inhibitor. AMG510, in KRAS^{G12C}-mutant PDAC (MIA PaCa2) and CRC(SW837) cells cultures in 3D (FIGS. 7A-7B). Although SDC1 membrane expression was quickly diminished upon mKRAS extinction in iKras cells (FIG. 7A) or KRAS^{G12C} inhibition with AMG510 in human PDAC (Miapaca2) and CRC (SW837) cells (FIG. 7B), it gradually reemerged following long-term mKRAS inhibition. Importantly, SDC1 was required for the tumorigenic activity of KRAS^{G12C} cells with acquired resistance to AMG510 (AMG510-R) (FIG. 8A). Moreover, ectopic expression of SDC1 in iKras tumor cells was able to maintain tumor growth upon extinction of KRAS^{G12D} in orthotopic xenograft model (FIG. 8B), indicating SDC1 is sufficient to bypass mKRAS-dependence.

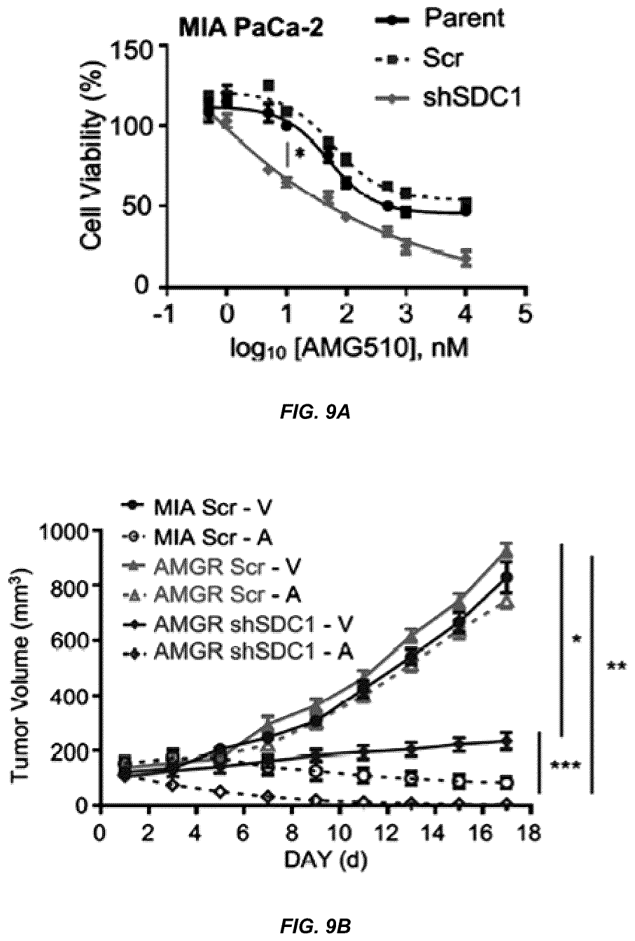

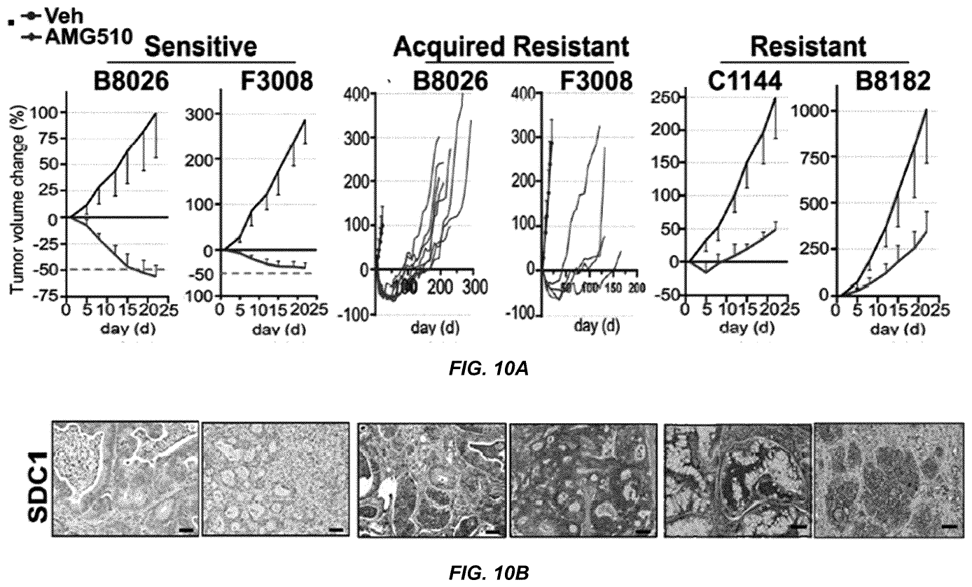

To further evaluate whether targeting SDC1 may sensitive KRAS-driven tumors to mKRAS targeted therapy, that the effect of genetic ablation of SDC1 was studied in viability assays of MIA PaCa2 cells harboring shScr or shSDC1 and treated with AMG510. The results show that genetic ablation of SDC1 significantly sensitized human MiaPaCa2 cells to AMG510 in vitro (FIG. 9A). Moreover, SDC1 knockdown also significantly blunted the in vivo growth of AMG510-resistant (AMG510)-R) xenograft tumors and led to complete tumor regression in combination with AMG510 treatment (FIG. 9B). Interestingly, analyzing the SDC1 expression level in CRC PDX models harboring KRAS^{G12C} demonstrated that AMG510-resistant PDX models exhibited higher SDC1 levels than the sensitive tumors (FIG. 10, bottom panel, right 2 images). Moreover, SDC1 expression levels in the sensitive PDX models (FIG. 10, bottom panel, left 2 images) were highly induced upon acquired resistance to AMG510 following long-term treatment (FIG. 10, bottom panel, middle 2 images), providing strong rationale to further evaluate the potential of co-targeting SDC1 and mKRAS in KRAS-driven tumors.

### Example 4. SDC1 is Required for the Macropinocytic Activity of Cells Resistant to mKRAS Inhibition

The ability to stimulate macropinocytosis, a regulated form of endocytosis, is a distinctive feature of mKRAS activation, and PDAC cells harboring mKRAS rely on increased levels of macropinocytosis for nutrient salvaging to sustain uncontrolled cell growth. A previous study demonstrated that the surface localization of SDC1 driven by mKRAS activation played a crucial role in maintaining macropinocytosis and tumor growth in PDAC (Yao, 2019). To better understand the function of SDC1 and the requirement of macropinocytosis for acquiring resistance to mKRAS inhibitors, the macropinocytic activity in mKRAS bypass tumors was examined. Although extinction of mKRAS leads to the rapid reduction of SDC1 surface expression and macropinocytosis, macropinocytosis levels in E-tumor cells were similar to that in E+ cells and iKras cells, suggesting that macropinocytosis is recovered in those escapers through a mKRAS-independent mechanism (data not shown). These data suggest that macropinocytosis is recovered in the E-cells possibly through mKRAS independent—but SDC1 dependent-mechanisms. Indeed, ablation of SDC1 abolished macropinocytic activity in E-cells and AMG510-resistant MIA PaCa-2 cells, indicating that SDC1 was required for macropinocytosis in these mKRAS-bypass cells (data not shown).

### Example 5. Development of Therapeutic Anti-SDC1 Monoclonal Antibody

The previous Examples above demonstrate that SDC1 is a viable therapeutic target for mKRAS-driven PDAC. Multiple strategies have been developed to target SDC1 due to its overexpression on multiple myeloma cells. Most notably, BT062-DM4 and B-B4-1131 are the same SDC1 targeting monoclonal antibody (clone BT062) but conjugated to the cytotoxic agent DM4 or a radioactive isotope, respectively. These therapeutic molecules are being investigated for treatment of multiple myeloma. However, functional antibodies that directly and specifically target the oncogenic function of surface SDC1 have not been developed. To generate SDC1 monoclonal antibodies, soluble recombinant human SDC1 (rhSDC1) was used to immunize Sdc1^{-/-} mice. The primary screening of the antibodies was by ELISA for binding to recombinant the rhSDC1 used for immunization, and multiple clones were in each well. From this initial screen, 47 wells were then screened by ELISA for binding to a His tag. From this primary screen, 19 wells (including multiple clones) were selected for a secondary screening and characterization process. First, single clones were isolated and confirmed by ELISA. Of these 14 single clones, 7 were found to react with human SDC1, 4 reacted with both human and mouse SDC1, and 3 reacted with both when at high coated levels. All 14 single clones were analyzed using FACS analysis, and 7 subclones were purified (226-10, 21A, 21B, 22B, 27, 28C, and 33A).

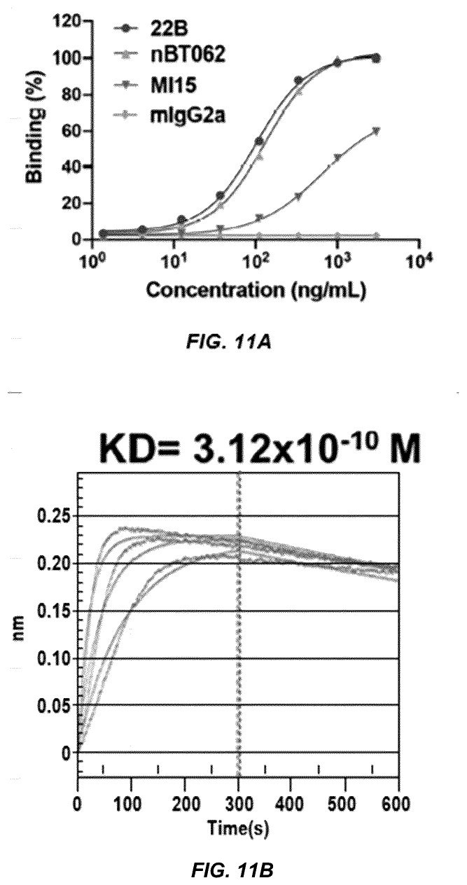

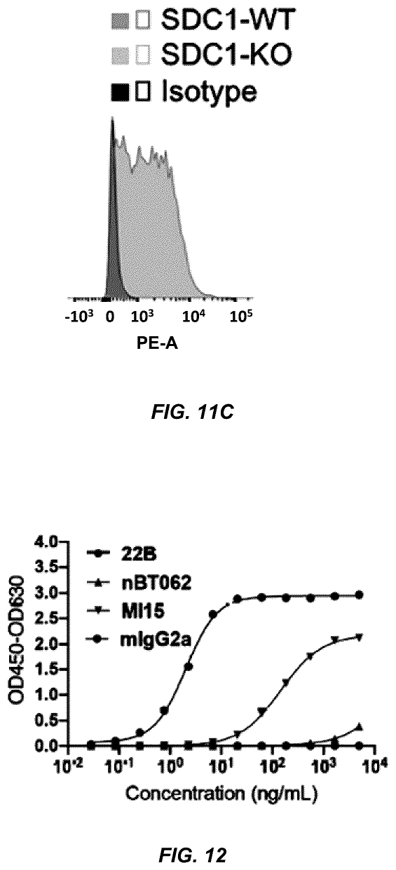

The in vitro binding affinities of clone 22B and the commercially available MI15 and nBT062 monoclonal antibodies to SDC1 were assessed by ELISA. The recombinant human SDC1 protein was plated onto 96-well plates. The plates were then incubated with purified 22B, MI15, or nBT062 and mglG2a. Plates were then washed and assessed for binding to the protein. When compared to the commercially available antibodies (FIG. 11A, triangles and inverted triangles), clone 22B demonstrated the highest binding affinity (FIG. 11A, dark circles). Importantly, clone 22B exhibited high sensitivity and specificity towards human SDC1 in PDAC cells, as seen in Table 4 and FIGS. 11B and 11C.

TABLE 4
Antibody binding affinity.

| Antibody | KD (M) | KD Error | Full R² |
|---|---|---|---|
| 226-10 | 9.76E−10 | 1.93 E−11 | 0.968 |
| 226-22B | 3.12 E−10 | 6.78 E−12 | 0.968 |

To further characterize the binding capability of clone 22B when compared to the nBT062 commercial antibody, IHC staining of normal human tissues (adrenal, bladder, bone marrow, brain, breast, cervix, colon, fallopian tube, kidney, liver, lung, myometrium, pancreas, placenta, prostate, salivary, skin, spleen, stomach, testis, thymus, thyroid, and tonsil) and PDAC-PDX models (data not shown). Tissues were fixed in 4% formaldehyde overnight at room temperature, moved to 70% ethanol for 48 hours, and then embedded in paraffin (Leica ASP300S). For immunohistochemistry, slides were deparaffinized in xylene and re-hydrated sequentially in ethanol. For antibodies requiring antigen retrieval, slides were treated with Citra-Plus Solution (BioGenex) according to manufacturer's instructions. Slides were quenched in 3% hydrogen peroxide activity to block endogenous peroxidase activity and then blocked in 10% FBS/5% BSA for 1 hour. Slides were incubated with primary antibodies and then secondary antibodies (ImmPress, Vector Lab) according to manufacturer's instructions. Nova RED (Vector Lab) or DAB (Abcam) were used for staining and images were captured with a Nikon DS-Fil digital camera using a wide-field Nikon EclipseCi microscope. The IHC staining demonstrated that clone 22B was capable of binding to SDC1 in cells at a comparable concentration to nBT062. In addition, 22B demonstrated a similar binding pattern to nBT062 in normal tissues (bladder, breast, pancreas, spleen, thymus) and in pancreatic tumors derived from established pancreatic ductal adenocarcinoma (PDAC) cells (AsPc1) and PDAC patient derived xenograft tumors (PATC66, PATC124, PATC153) (data not shown). Clone 27 exhibited similar binding properties (data not shown). Further evaluation of 22B binding affinity demonstrated that 22B has a higher binding affinity to cynomolgus SDC1 protein when compared to nBT062 and MI15 commercial antibodies (FIG. 12), underscoring their translational utility.

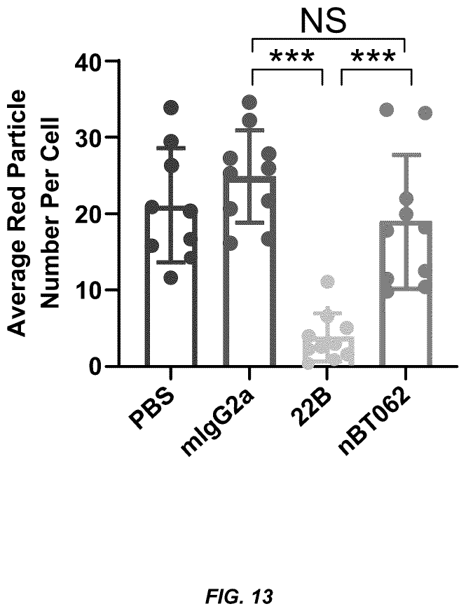

Given the critical role of SDC1-mediated macropinocytosis of PDAC biology, the 22B antibody and commercially available nBT062 were analyzed for their effectiveness on inhibiting macropinocytosis (FIG. 13 and data not shown). Human PDAC cells, PATC53 cells, were seeded in 8-well chamber slides (LabTek). After cell attachment, cells were treated with PBS, mIgG2a, clone 22B or nBT062 and switched to serum-free medium for 18 hours. Macropinosomes were marked utilizing a high molecular weight TMR-dextran (Fina Biosolutions) uptake assay in which TMR-dextran was added to serum-free medium at a final concentration of 1 mg/ml for 35 minutes at 37° C. At the end of the incubation period, cells were rinsed five times in cold phosphate buffer saline (PBS) and immediately fixed in 4% paraformaldehyde. Cells were DAPI-treated to stain nuclei, and coverslips were mounted onto slides using DAKO mounting medium (DAKO). Images were captured using FV1000 Olympus Confocal Microscope system (data not shown) and analyzed using the ‘Analyze Particles’ feature in ImageJ (NIH). The total particle area per cell was determined from at least 6 fields that were randomly selected from different regions across the entirety of each sample. 22B, but not nBT062 treatment, significantly reduced macropinocytosis as indicated with decrease in TMR-dextran signal in 22B-treated cells, compared to nBT062 or mIgG2a isotype treated cells. This experiment showed that 22B was capable of inhibiting macropincytosis while clone nBT062 exhibited no impact (FIG. 13 and data not shown). Thus, clone 22B is the first anti-SDC1 monoclonal antibody that can directly suppress SDC1 function.

### Example 6. Optimization of Anti-SDC1 Monoclonal Antibody Mediated Antibody-Dependent Cellular Cytotoxicity (ADCC)

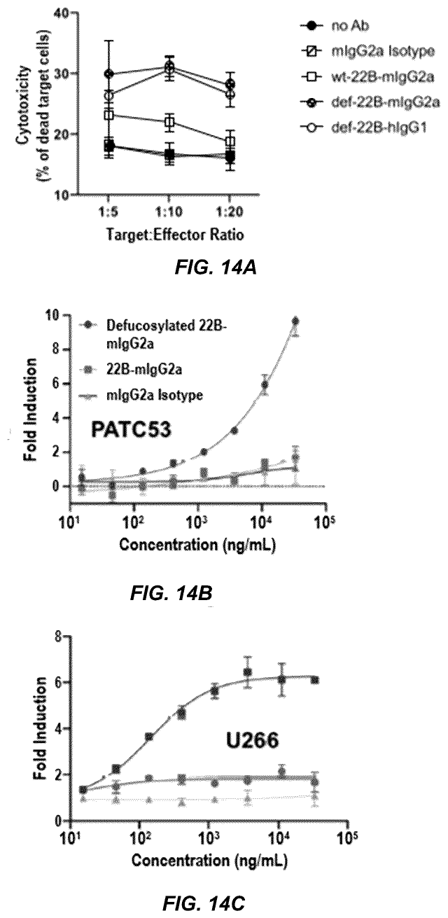

In addition to the direct effects on tumor cells, the anti-tumor effect of monoclonal antibodies may also rely on their ability to induce various cytotoxic machineries against specific targets. These cytotoxic machineries include antibody-dependent cellular cytotoxicity (ADCC), antibody-dependent cellular phagocytosis (ADCP), and complement-dependent cytotoxicity (CDC), which are all mediated via the fragment crystallizable (Fc) domain of monoclonal antibodies. The biological activity of the lead clone (22B) against hSDC1 overexpressed Panc02 cells was investigated with respect to Fc-mediated effector functions, hSDC1 overexpressed Panc02 cells were used as a target, and NK92-CD16 cells were the effector cells. The results showed that there was no significant CDC or ADCP induced by clone 22B while there was significant, although weak, ADCC, with higher dead cell ratio upon 22B treatment, which is further enhanced by defucosylation (FIG. 14A). ADCC is mediated by the Fc domain of the monoclonal antibody which binds to the Fc receptors on effector cells, such as NK cells.

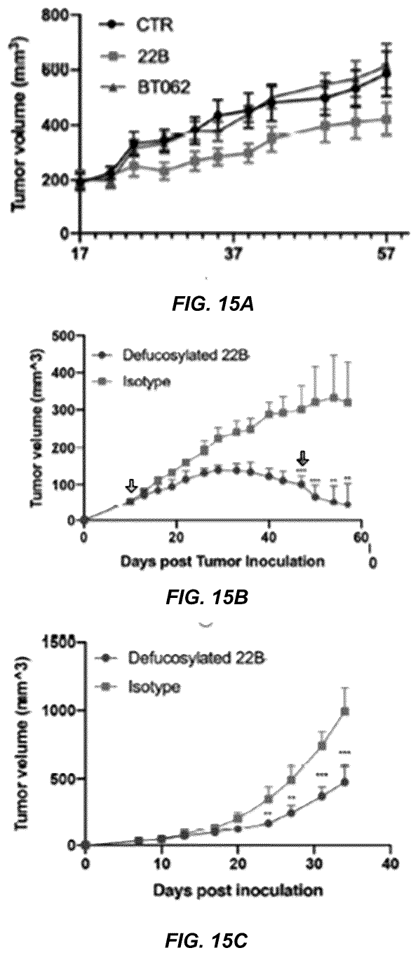

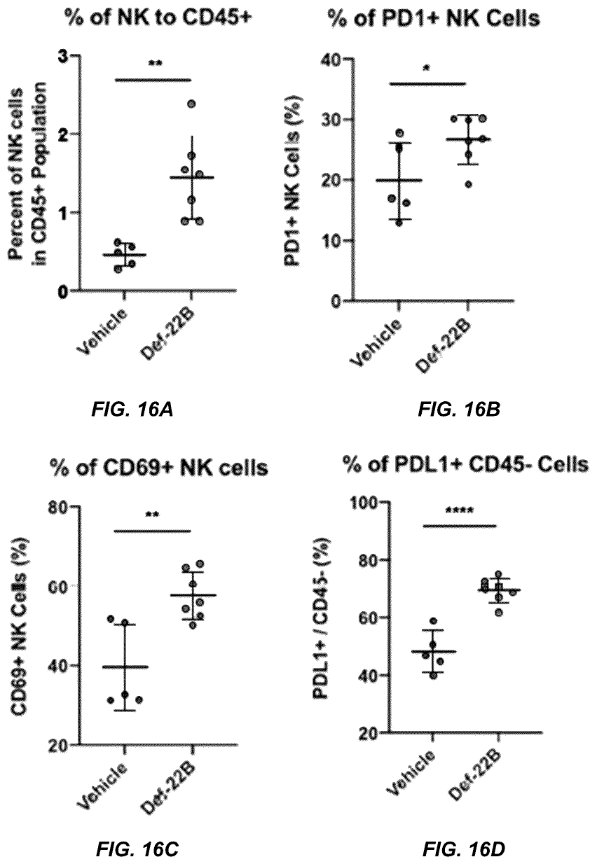

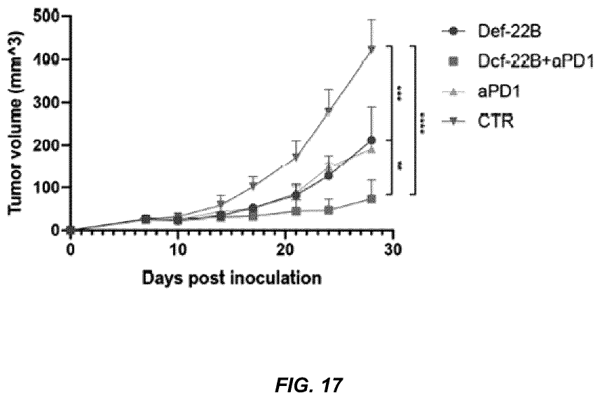

It has been reported that the presence of fucose on the core glycan structure in the antibody Fc region is negatively correlated with the binding affinity with FcγRIIIa receptor on immune effector cells. Therefore, a completely non-fucosylated 22B antibody was produced. First. CRISPR-mediated deletion of the α1,6-fucosyltransferase gene (encoding FUT8) in Chinese hamster ovary (CHO) cells was performed. The resulting cells were then used to produce a defucosylated version 22B antibody. The 22B antibody and defucosylated 22B were analyzed for their effect on ADCC in PATC53 and B-lymphocyte (U266) cells. Defucosylation of the 22B antibody greatly improved the ADCC effect in both cell lines (FIGS. 14B-14C). Importantly, the defucosylation of 22B led to dramatic tumor regression in the SQ xenograft model of AsPC1 in nude mice, which still maintain functional innate immunity (FIG. 15B). In contrast, wild-type 22B (FIG. 15A, squares) showed mild anti-tumor efficacy while commercial anti-SDC1 BT062 monoclonal antibody exhibited no direct anti-tumor effect (FIG. 15A, triangles). In addition, defucosylated 22B also significantly suppressed the growth of syngeneic SQ xenograft tumors derived from Panc02 mouse PDAC cells expressing human SDC1 (Panc02-hSDC1) (FIG. 15C). The SQ tumors derived from Panc02-hSDC1 were further subjected to CD45. NK1.1. CD69. PD1, and PD-L1 staining and FACS analysis. Consistent with the strong ADCC activity of defucosylated 22B, total NK cells and activated CD69+NK cells were significantly induced in tumor microenvironment following defucosylated 22B treatment (FIGS. 16A-16B). Interestingly. PD-1 was significantly induced in NK cells accompanied with concurrent PD-L1 upregulation in tumor cells following defucosylated 22B treatment (FIGS. 16C-16D), indicating the activation of immune checkpoint to curb NK cell activation. Importantly, treatment with anti PD1 monoclonal antibody showed strong cooperation with 22B to abolish tumor growth in Panc02-hSDC1 cells (FIG. 17). These data suggest that the optimized 22B can harness immune system to elicit strong ADCC activity and synergize with immune checkpoint therapy to enhance the anti-tumor efficacy.

### Example 7. Internalization of Anti-SDC1 Antibody by PDAC

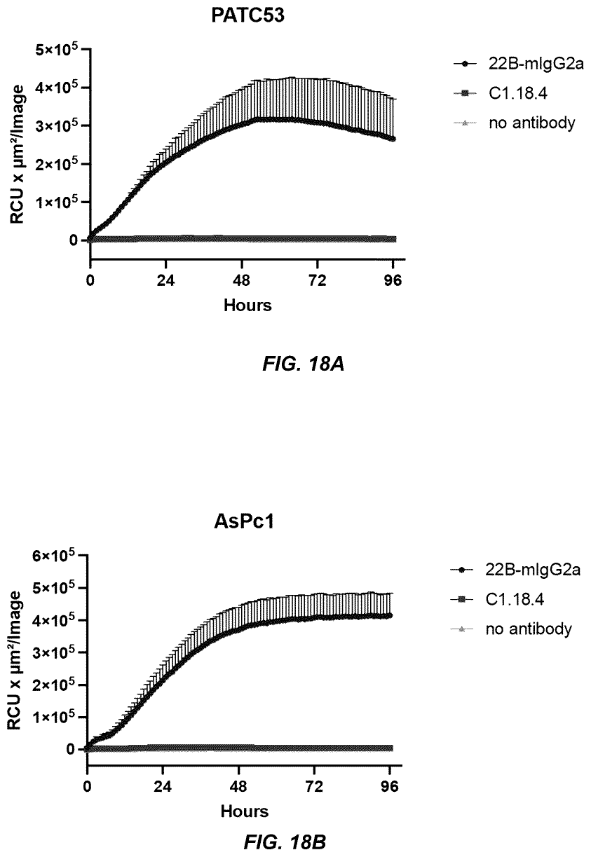

To further analyze the 22B antibody, internalization of the antibody was examined by Incucyte (FIGS. 18A-18B). Serial diluted 22B antibody (circles), mouse IgG2a isotype control (squares), or no antibodies (triangles) were mixed with FabFluor-pH dye and incubated with PATC53 or AsPc1 cells for 0-96 hours, and the fluorescence was detected. Antibodies that were internalized and entered into a lysosome showed red fluorescence (measured as RCU). FIGS. 18A-18B show the results, with error bars, where 4 μg/mL of antibody was used for the time indicated on the x axis. The data demonstrates that the 22B antibody is internalized by both PATC53 cells (FIG. 18A) and AsPc1 cells (FIG. 18B).

### Example 8. Anti-SDC1 Antibody Suppresses Tumor Progression in PDAC Models

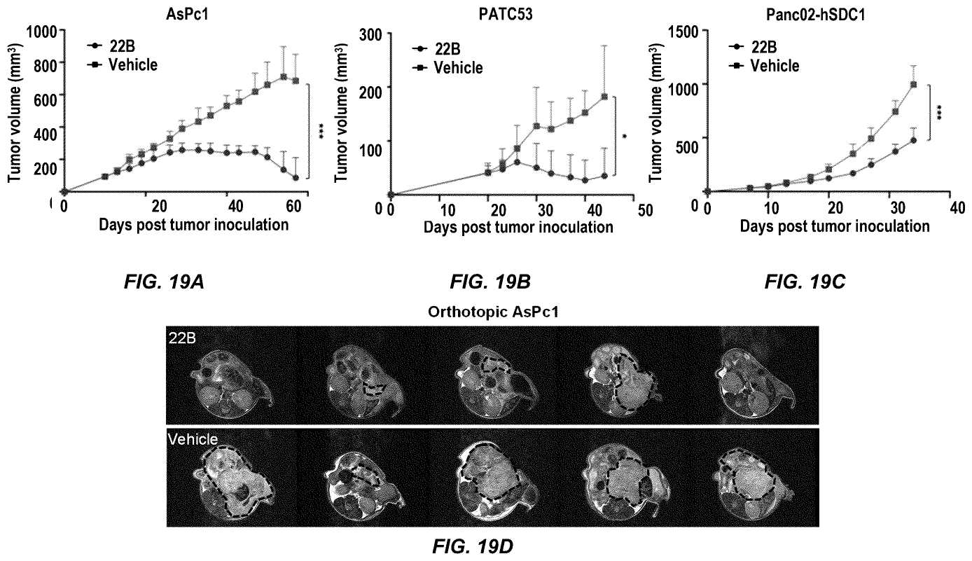

To analyze the ability of the 22B antibody to suppress tumor progression, the antibody was used in several PDAC models (FIGS. 19A-19D). Subcutaneous xenograft tumors derived from established PDAC cell line. AsPc1 in nude mice (FIG. 19A), or PDAC patient derived cells. PATC53 in nude mice (FIG. 19B), or mouse PDAC cell line Pan02 expressing human SDC1 in C57BL/6NJ mice (Panc02-hSDC1) (FIG. 19C), were treated with 22B or an IgG2a control when tumors reached 50-100 mm³. The tumor volume was then measured over a period of days as shown in FIGS. 19A-19C. The 22B demonstrated the ability to suppress the tumor growth (circles), while the tumors in the mice treated with the control antibody continued to grow (squares).

The ability of 22B to suppress tumor progression was further analyzed using orthotopic xenograft tumors derived from AsPc1 in nude mice (FIG. 19D). The orthotopic xenograft tumors were treated with 22B (top panel) or the IgG2a control antibody (bottom panel), and the tumors were imaged by MRI. The results further demonstrated the ability of 22B to suppress tumor growth.

### Example 9. Combination Therapies Enhance the Effect of the Anti-SDC1 Antibody

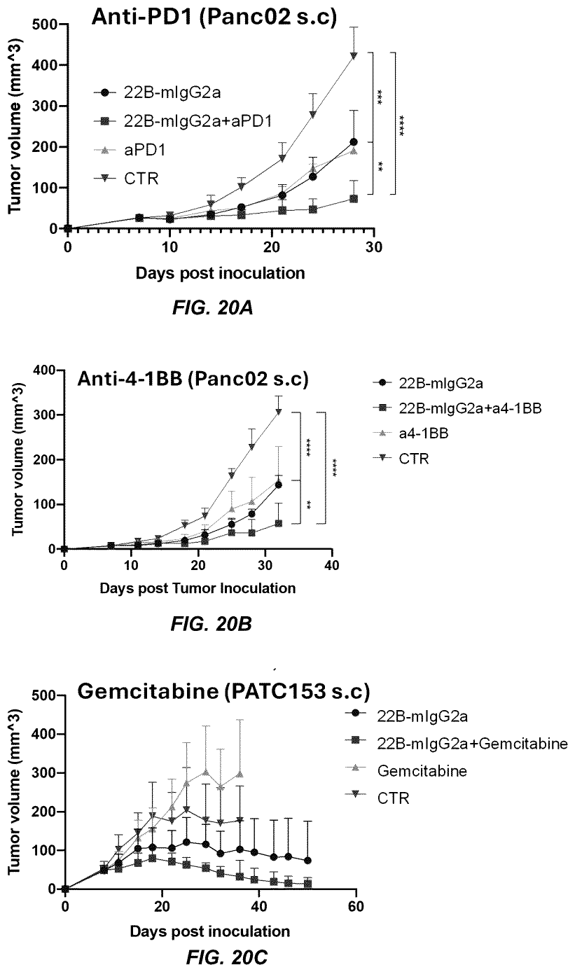

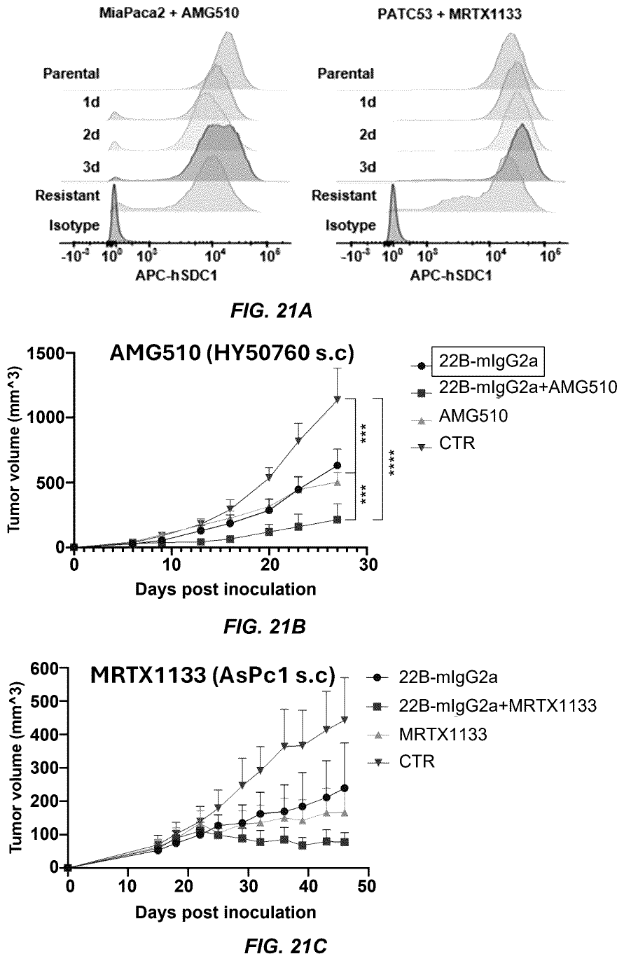

To investigate whether the tumor-suppressive activity of the 22B antibody could be enhanced, various antibodies were analyzed in combination with 22B (FIGS. 20A-20C, 21A-21C). Subcutaneous xenograft tumors derived from mouse PDAC cell line Pan02 expressing human SDC1 in C57BL/6NJ mice were treated with the 22B antibody (circles), a PD1 antibody (triangles), a combination of 22B and PD1 antibodies (squares), or a control antibody (inverted triangles) when tumors reached around 20 mm³ (FIG. 20A). The results demonstrate that the addition of a PD1 antibody enhanced the tumor suppressive activity of the 22B antibody. Similarly, subcutaneous xenograft tumors derived from mouse PDAC cell line Pan02 expressing human SDC1 in C57BL/6NJ mice were treated with the 22B antibody, a 4-1BB antibody, a combination of 22B and 4-1BB antibodies, or a control antibody (FIG. 20B). The results demonstrate that the addition of a 4-1BB antibody enhanced the tumor suppressive activity of the 22B antibody. In another experiment, subcutaneous xenograft tumors derived from human patient derived PDAC cells PATC153 in nude mice were treated with the 22B antibody. Gemcitabine, a combination of 22B and Gemcitabine, or a control antibody (FIG. 20C). The results demonstrate that the addition of Gemcitabine enhanced the tumor suppressive activity of the 22B antibody.

Kras inhibitors were analyzed to determine if they had an effect on the tumor-suppressive activity of 22B (FIGS. 21A-21C). First, the SDC1 expression level was measured by FACS in MiaPaca1 PDAC cells or PATC53 PDAC cells after treatment with the Kras inhibitor AMG510 or MRTX1133. The SDC1 expression level was decreased upon acute treatment with Kras inhibitor, AMG510, for 1 day and 2 days, but recovered upon long term treatment for longer than 3 days (FIG. 21A, left panel). The SDC1 expression level in PATC53 PDAC cells remains high upon treatment with Kras inhibitor, MRTX1133 (FIG. 21B, right panel). These data suggest that SDC1 high expression is a mechanism of resistance to Kras targeted therapy and that the combination of 22b and Kras inhibitor may confer better anti-tumor efficacy in Kras mutated human cancers including PDAC, lung cancer, colorectal cancer, and others.

Subcutaneous xenograft tumors derived from mouse Kras^{G12C}-driven PDAC cell line, HY50760 expressing human SDC1 in C57BL/6NJ mice were treated with the 22B antibody, AMG510, a combination of 22B and AMG510, or a control antibody. The results demonstrate that the addition of AMG510 enhanced the tumor suppressive activity of the 22B antibody. Similarly, subcutaneous xenograft tumors derived from human PDAC cell line AsPc1 in nude mice were treated with the 22B antibody, MRTX1133, a combination of 22B and MRTX1133, or a control antibody. The results demonstrate that the addition of MRTX1133 enhanced the tumor suppressive activity of the 22B antibody.

### Example 10. The Anti-SDC1 Antibody Targets a Novel SDC1 Epitope

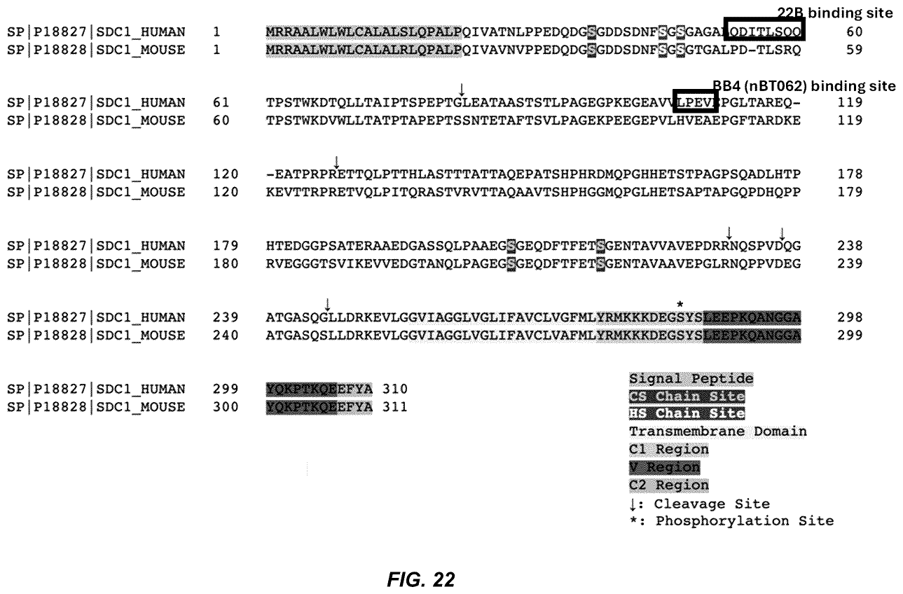

To identify the exact SDC1 epitope that the 22B antibody binds, a single amino acid resolution, conformational epitope mapping was performed. Epitope mapping discovered two potential binding sites of clone 22B (data not shown). The potential epitopes included DITLSQ (SEQ ID NO: 22) and DFTF (SEQ ID NO: 25); however, stronger binding of the 22B antibody to longer peptides containing DITLSQ (SEQ ID NO: 22) was observed, but not to longer peptides containing DFTF (data not shown). Flow cytometry analysis demonstrated the binding of the 22B antibody to hSDC1 lacking QDFTF, and demonstrated no binding of the 22B antibody to hSDC1 lacking DITLSQ (data not shown). These data confirmed that the epitope of 22B is DITLSQ. Protein sequence alignment of human and mouse SDC1 revealed that the binding region of 22B is close to the heparin sulfate chain binding sites (red labeled “S”) on the hSDC1 extracellular domain (FIG. 22).

### Example 11. Methods and Materials

### Binding and Internalization

The radiolabeled antibody, [⁸⁹Zr]Zr-DFO-22B, was generated according to a reported procedure with minor modifications. (Raave et al., Eur. J. Nucl. Med. Mol. Imaging 46, 1966-1977 (2019)). Cells (SDC1+: AsPC-1 cells; or SDC1−: AsPC-1 cells, SDC1 knockout (KO)) were harvested and suspended in a medium containing 1% bovine serum albumin (BSA) at a concentration of 1×10⁶ cells/100 μL and subsequently incubated with 250 pM [⁸⁹Zr]Zr-DFO-22B or [⁸⁹Zr]Zr-DFO-non-targeting IgG for 2 hours. Nonspecific binding and internalization were determined by co-incubation with 500 nM unlabeled 22B. Cells were centrifuged at 300×g for 5 minutes and washed twice with ice-cold phosphate buffer saline (PBS). The membrane-bound fraction was removed from the cells by incubation in an acidic solution (pH 2.6) at 4° C. for 5 minutes. Binding and internalization were determined by counting the radioactivity using a gamma counter (HIDEX, LabLogic).

### Small Animal PET CT Imaging in Mouse Model of PDAC

To prepare the mouse xenograft models, a suspension of PATC53 cells (5×10⁶) or AsPC-1 SDC1 KO cells (2×10⁶) in 100 μL of 1:1 PBS/Matrigel was subcutaneously injected into the flank of 6-8 weeks old male athymic nude mice provided from The Jackson Laboratory. Groups of four or five mice bearing palpable PATC53 or AsPC-1 SDC1 KO tumors were injected with 1.22±0.11 MBq (32.9±3.02 μCi) of radioimmunoconjugate via the tail vein. Four treatment cohorts of PATC53±[⁸⁹Zr]Zr-DFO-22B, AsPC-1 SDC1 KO+[⁸⁹Zr]Zr-DFO-22B, PATC53±[⁸⁹Zr]Zr-DFO-non targeting IgG, and PATC53±[⁸⁹Zr]Zr-DFO-22B with an additional injection of 350 μg 22B were compared to demonstrate the specificity of 89Zr-labeled 22B on the overall PET signal (n=2). PET images were taken 24, 48, and 120 hours post injection. After anesthetization by isoflurane inhalation (2.5% in an air mixture), mice were imaged with a small-animal PET scanner (Transaxial FOV: 10 cm, axial FOV: 12.7 cm, resolution at the center of FOV: 1.4 mm, Bruker Albira PET/SPECT/CT scanner, Billerica, MA). MicroPET/CT images were acquired over a period of 20-30 minutes. In all studies, PET scanning was performed via placing animals over a heating pad maintained at 37° C. After the PET scan, mice were euthanized by carbon dioxide inhalation, and tissues of interest were excised for biodistribution experiments. The energy window was set between 350 and 650 keV. The imaging data were reconstructed using a 3-D ordered-subsets expectation maximization algorithm. The CT images were obtained using a three-dimension-based, filtered back-projection algorithm. The fusion of PET and CT images was performed using PMOD base functionality software version 4.0 (PMOD Technologies LLC, Zurich, Switzerland) as well as Imalytics Preclinical software (Gremse-IT). For quantification, the volumes-of-interest (VOIs) were manually drawn to record the mean radioactivity and convert the values to % ID/cc.

### In Vivo Biodistribution of [⁸⁹Zr]Zr-DFO-22B

After the final imaging session, animals were immediately euthanized by carbon dioxide inhalation. Blood, tumor, and other organs of interest were collected and weighed. Tumors were cut in half and embedded into optimal cutting temperature (OCT) compounds for subsequent autoradiography and immunofluorescent staining. Radioactivity content in the blood and each tissue were counted using a gamma counter (HIDEX, LabLogic) and expressed as % injected dose per gram of tissue (% ID/g).

### Digital Autoradiography

Tumor tissues were flash frozen in OCT compounds in dry ice. The tissues were sectioned with a microtome (Leica) into slices having a thickness of 10-20 μm and directly mounted on glass slides (VWR). A GE Amersham Imaging Plate was exposed by such slides with radioactive tissue. The screen was developed on a phosphorimager (Typhoon 5). The images were further analyzed by using ImageQuantTL software.

### Dose Escalation Study

Groups of three male athymic nude mice (6-8 weeks old, The Jackson Laboratory) were treated intravenously with escalating doses of [¹⁶¹Tb]Tb-DO3A-22B (150, 200, 250, 300 μCi; each 30 μg 22B). After injection, mice were weighed every other day and monitored daily for humane endpoint. The maximum tolerated dose (MTD) is defined as the first dose level below the dose leading to >20% decrease in total body weight in at least one of the mice, or an early reaching of a defined endpoint of at least one mouse. Blood chemistry was analyzed 21 days post i.v. injection of radioimmunoconjugates in blood serum at the MD Anderson Veterinary Medicine & Surgery Core Facility.

### Treatment Study in a Xenograft Mouse Model of Pancreatic Cancer

The AsPC-1 tumor model was prepared as described above. Once the tumors reached an average volume of 218 mm³, the mice were randomly assigned to four to five separate groups and injected intraperitoneally with sterile-filtered human IgG (30 mg/kg) in PBS. 24 hours later, a single dose of [¹⁶¹Tb]Tb-DO3A-22B (100 or 200 μCi; each 30 μg 22B), [¹⁶¹Tb]Tb-DO3A-non targeting IgG (100 μCi: 3 μg IgG), or PBS (no-treatment control) was administered to mice intravenously. Tumor volume (0.52×a×b², a: long diameter, b: short diameter) and body weight were monitored twice a week. Mice were euthanized when the tumor volume exceeded 1000 mm³, the tumor size exceeded 2 cm in diameter, the tumor ulceration exceeded 4 mm, or the mice showed severe signs of distress. Such events were counted as deaths.

### Statistical Analysis

Statistical analysis was performed with GraphPad Prism® 8.0 software (GraphPad Software). For the biodistribution studies, a Welch's t-test (two-tailed, unpaired, uneven variance) was used. One-way ANOVA with Dunnett's multiple comparisons test was used for the comparison of the tumor size in the treatment studies. Kaplan-Meier survival curve statistics were analyzed with a log rank (Mantel-Cox) test. Differences with p values less than 0.05 were considered statistically significant in all analysis.

### Example 12. Analysis of [⁸⁹Zr]Zr-DFO-22B as First Agent in SDC1 Theranostic Pair

Antibodies represent attractive carriers for the development of high-affinity, highly selective theranostic pairs, offering precision in cancer cell targeting from visible, solid tumors to micrometastatic lesions. In prior studies, a lead monoclonal antibody, “22B,” targeting human SDC1 was identified that had high affinity and selectivity, along with the ability to rapidly internalize and suppress micropinocytosis activity in SDC1 overexpressing PDAC cells, suggesting its suitability as the basis for a theranostic drug development campaign. Utilizing 22B as an SDC1-targeted carrier, zirconium-89 (⁸⁹Zr) and terbium-161 (¹⁶¹Tb)-labeled 22B were generated in a site selective manner.

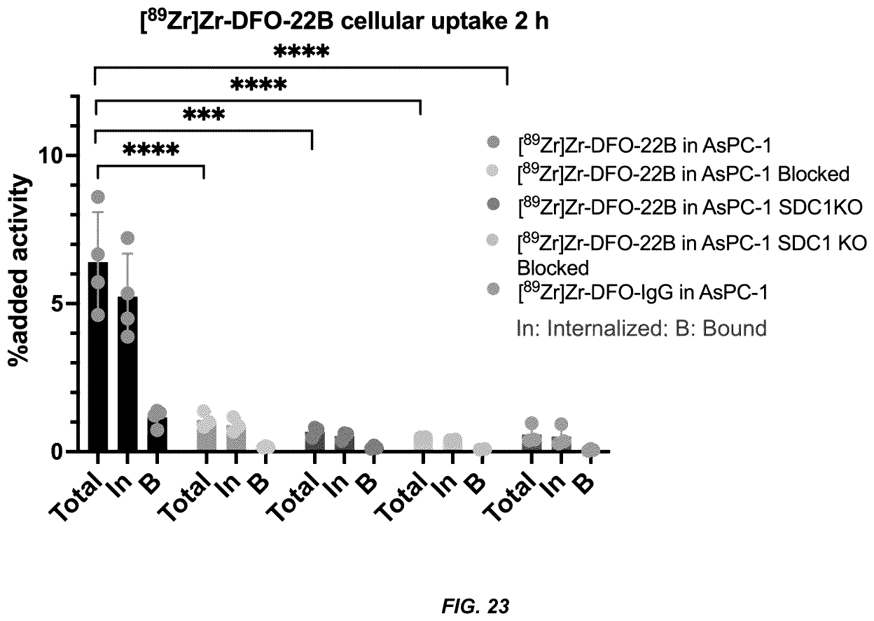

Experiments were conducted to determine the binding and internalization of the [⁸⁹Zr]-labeled antibody. Specifically, the in vivo cellular uptake of [⁸⁹Zr]-labeled antibodies demonstrated rapid internalization and cellular uptake upon incubation with SDC1+ AsPC-1 cells (FIG. 23). Cellular uptake of [⁸⁹Zr]Zr-DFO-22B in SDC1+ AsPC-1 cells was 6.40±1.69% at 2 hours, with 81.7±2.76% internalization rate. The uptake was significantly lower for the SDC1-AsPC-SDC1 knockout cells (0.67±0.19%, P<0.001). Incubation with [89Zr]Zr-DFO-IgG or incubation with an excess amount of nonradioactive 22B resulted in significant reduction of radioactivity uptake (P<0.001), suggesting potential antibody directed delivery to the target tissue.

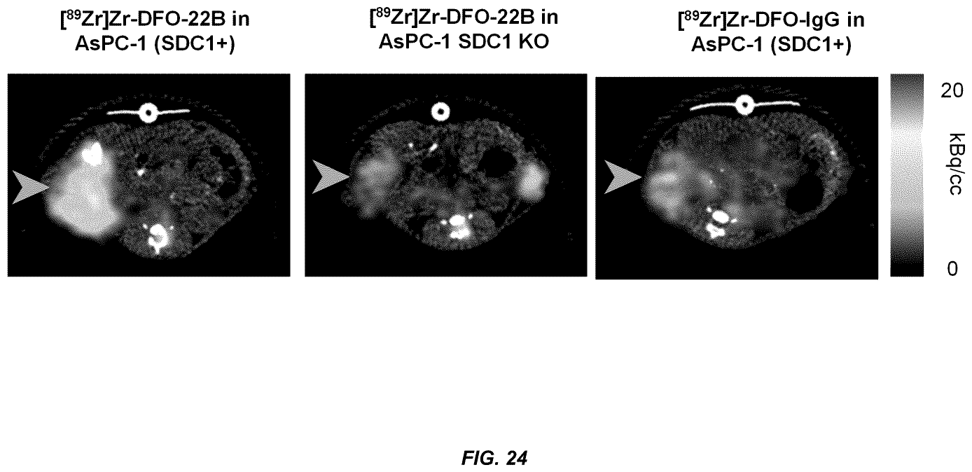

In vivo PET analysis of AsPC-1 PDAC xenografts with [89Zr]Zr-DFO-22B confirmed the cellular uptake following injection of the [⁸⁹Zr]-labeled antibodies (FIG. 24). The SDC1+ xenograft (left image) demonstrated a significant uptake of the [89Zr]Zr-DFO-22B antibody as compared to the SDC1 KO xenograft (middle image). In addition, the [⁸⁹Zr]Zr-DFO-22B demonstrated an increased uptake in the xenograft tumor as compared to the control antibody presenting [⁸⁹Zr]Zr-DFO-IgG, suggesting that the site-specific localization in the tumor may be mediated by the 22B antibody.

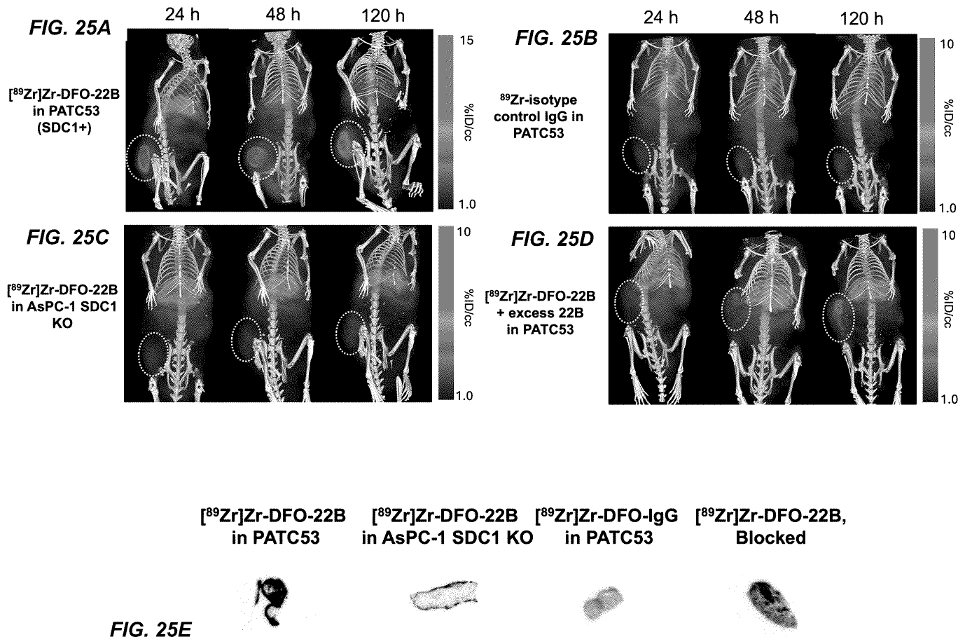

Further analysis of the localization of the radionuclide moiety in the tumor tissue via in vivo microPET/CT imaging revealed clear and persistent visualization of SDC1-positive PATC53 tumors. For this experiment, athymic nude mice bearing subcutaneous PDAC cell line xenograft tumors (from SDC1+ PATC53 or AsPC-1 SDC1 KO cells) were administered [⁸⁹Zr]Zr-DFO-22B, [⁸⁹Zr]Zr-DFO-IgG control antibody, or [⁸⁹Zr]Zr-DFO-22B in combination with excess unlabeled 22B. Images were taken at 24, 48, and 120 hours post injection for each mouse. Administration of [⁸⁹Zr]Zr-DFO-22B resulted in clear and persistent visualization of SDC1-positive PATC53 tumors up to 120 hours post-injection (FIG. 25A). Co-administration of excess amount of nonradioactive 22B or administration of [⁸⁹Zr]Zr-DFO-IgG resulted in visibly less uptake in the SDC1+22B tumor (FIG. 3D), suggesting SDC1-mediated [⁸⁹Zr]Zr-DFO-22B uptake in PATC53 tumor. The tumors lacking SDC1 expression were not able to be visualized with [⁸⁹Zr]Zr-DFO-22B, and the SDC1+ tumors were not able to be visualized with the [⁸⁹Zr]Zr-DFO-IgG control antibody (FIGS. 25B-25C), further demonstrating the importance of SDC1 in tumor visualization with the 22B radionuclide conjugate. Further analysis was performed via ex vivo autoradiography of the excised tumors (FIG. 25E) from each of the xenograft models in FIGS. 25A-25D. The autoradiography images further confirm the intense radioactivity uptake of [89Zr]Zr-DFO-22B specifically in SDC1-positive PATC53 tumors.

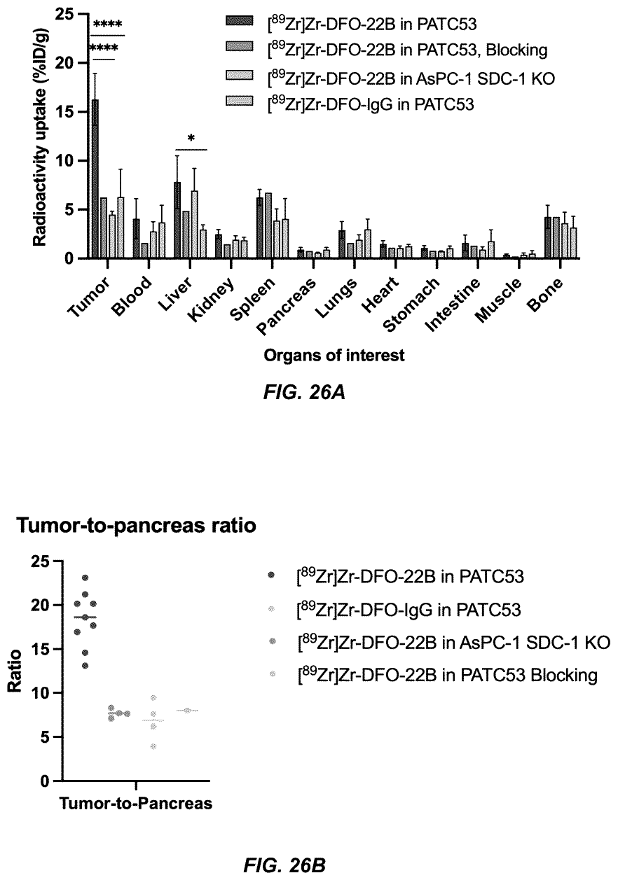

Additional analysis of cellular uptake was evaluated via in vivo biodistribution analysis (FIG. 26A). The experiment was carried out via the methods described above. Gamma counting of the excised tumor and the organs of interest was performed to quantify the radioactivity uptake immediately after the final PET/CT images were collected in FIG. 25A. The radioactivity uptake in the PATC53 tumor injected with [⁸⁹Zr]Zr-DFO-22B (18.49±3.68% ID/g) was significantly greater than those of PATC53 tumor injected with [⁸⁹Zr]Zr-DFO-IgG control or SDC1-AsPC-1 SDC1 KO tumor injected with [⁸⁹Zr]Zr-DFO-22B (4.81±1.61% ID/g and 6.51±2.91% ID/g, respectively, P<0.0001). PATC53 tumors demonstrated highest [⁸⁹Zr]Zr-DFO-22B uptake amongst any other organs, further confirming the cellular uptake observed in the PET/CT imaging. Comparing the tumor distribution to that of the pancreas, a greater than 20-fold increase in accumulation was observed. These results taken collectively, demonstrate the potential of [⁸⁹Zr]Zr-DFO-22B for selectively accumulating to pancreatic cancers with high tumor-to-background contrast (FIG. 26B).

### Example 13. Analysis of [¹⁶¹Tb]Tb-DO3A-22B as Second Agent in SDC1 Theranostic Pair

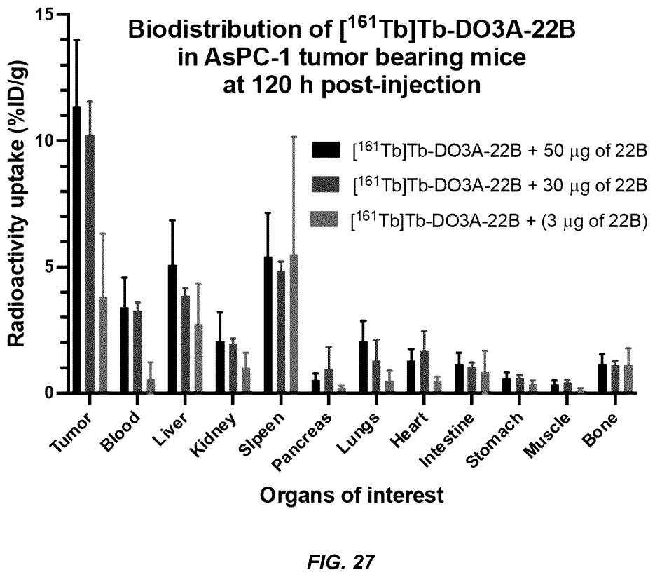

Encouraged by the imaging characteristics of [89Zr]Zr-DFO-22B, a therapeutic counterpart, [¹⁶¹Tb]Zr-DO3A-22B, was produced. The in vivo profile and treatment efficacy of [¹⁶¹Tb]Zr-DO3A-22B was evaluated in an AsPC-1 xenograft mouse model. Specifically, athymic nude mice bearing subcutaneous AsPC-1 SDC1+ xenograft tumors were administered [¹⁶¹Tb]Tb-DO3A-22B. Then 120 hours post injection, the mice were euthanized, and the organs of interest were excised from the mice. Gamma counting of the excised tumor and the organs of interest was performed to quantify the radioactivity uptake. The radioactivity uptake in the tumor injected with [¹⁶¹Tb]Tb-DO3A-22B (12.0±3.0% ID/g) was greater than healthy organs. Cellular uptake of the [¹⁶¹Tb]-labeled antibody demonstrated a dose dependent response (FIG. 27). Comparing the tumor distribution to that of the liver, a greater than 2-fold increase in accumulation was observed in the tumor tissue. These results taken collectively, demonstrate the potential of [¹⁶¹Tb]Tb-DO3A-22B for selectively accumulating to liver metastasis with high tumor-to-background contrast (FIG. 27).

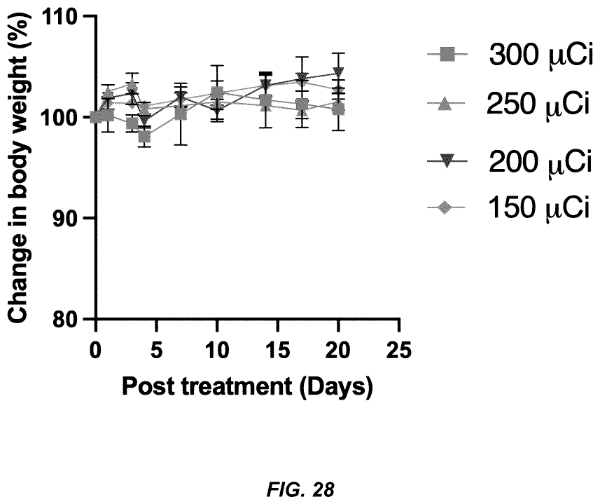

Experiments were conducted to determine the therapeutic effect of [¹⁶¹Tb]Tb-DO3A-22B. In these experiments, groups of three athymic mice were treated with varying concentrations (150, 200, 250, or 300 μCi) of [¹⁶¹Tb]Tb-DO3A-22B, and the weight was assessed every day for 21 days (FIG. 28). The amount of the injected radioimmunoconjugate was adjusted to 30 μg for each athymic mouse. Within 21 days after administration of the radioimmunoconjugate, there was no observed toxicity related to the application of the radioimmunoconjugate (no signs of distress, no change in behavior, and no radiation syndrome). Blood chemistry data demonstrated a slight increase in liver enzymes such as ALT and AST in mice treated with greater than 200 μCi of [¹⁶¹Tb]Tb-DO3A-22B (data not shown). The histopathological analysis of tissues revealed slight morphological change in the liver. Additionally, the ALT and AST levels returned to normal range when blood chemistry data were analyzed at 53 days post-treatment of 200 μCi of [¹⁶¹Tb]Tb-DO3A-22B, suggesting adverse effect on liver may be transient and recovery may be possible upon discontinuation of the treatment. The data demonstrates that up to 300 μCi of [¹⁶¹Tb]Tb-DO3A-22B can be safely administered in the further efficacy evaluation studies.

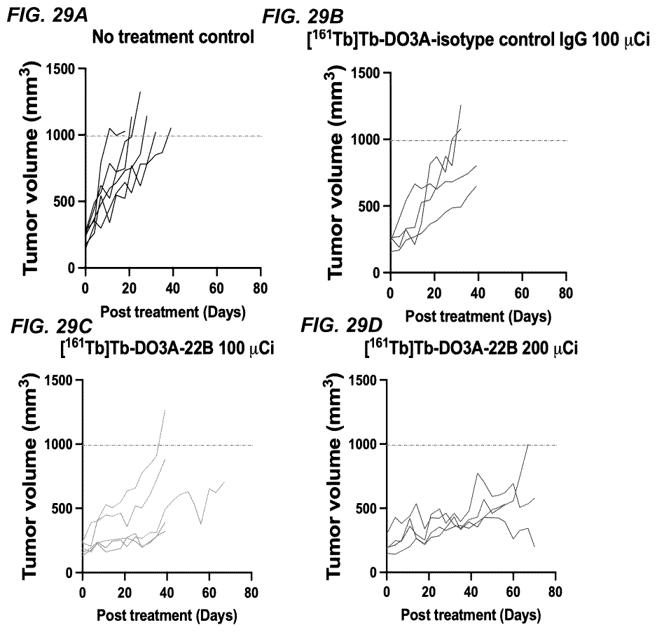

Additional experiments in xenograft mouse models of pancreatic cancer confirmed treatment efficacy of the 22B radioimmunoconjugate (FIG. 29A-29D, 30A-30C). To prevent fast clearance of the immunoconjugates, tumor-bearing mice were preconditioned by intraperitoneal administration of human IgGs (30 mg/kg) one day prior to the initiation of the treatment. Once the tumor volume reached greater than 200 mm³, the mice were intravenously injected with a single dose of [¹⁶¹Tb]Tb-DO3A-22B (100 or 200 μCi; each 30 μg 22B (FIGS. 29C and 29D, respectively), [¹⁶¹Tb]Tb-DO3A-non targeting IgG (100 μCi: 30 μg IgG) (FIG. 29B), or PBS (FIG. 29A). The control, non-treatment group, reached the 1000 mm³ threshold in under 40 days. The [¹⁶¹Tb]Tb-DO3A-non targeting IgG demonstrated only limited suppression of tumor growth. However, the xenograft mice treated with [¹⁶¹Tb]Tb-DO3A-22B (100 or 200 μCi; each 30 μg 22B) demonstrated a slowed to no tumor growth after 60 days post treatment.

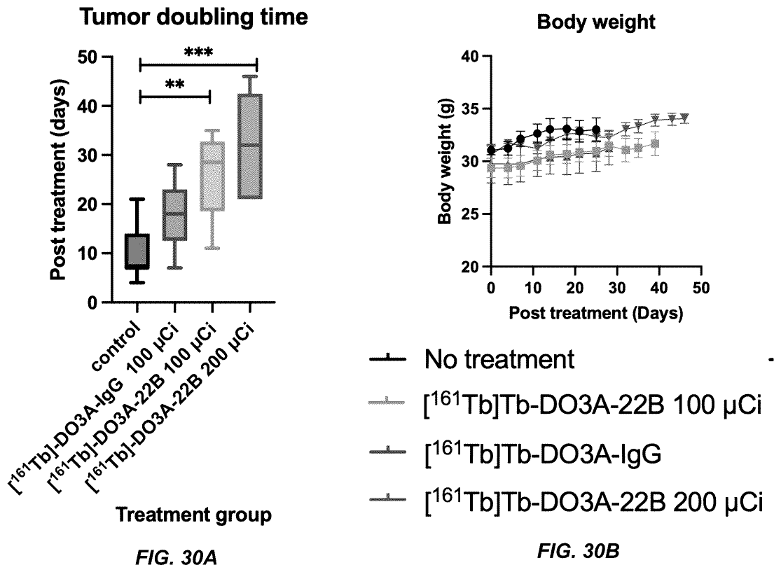

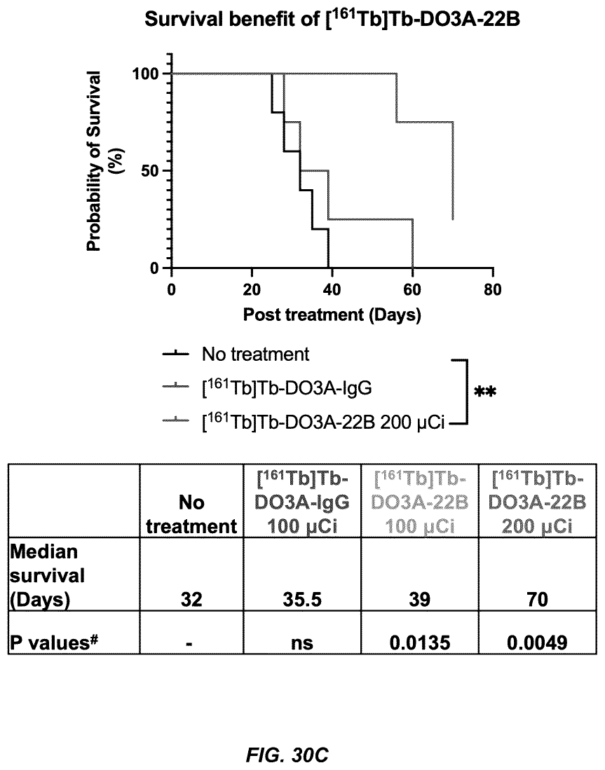

To further demonstrate these findings, tumor doubling time was assessed (FIG. 30A). The control (no treatment) group demonstrated a tumor doubling time of about 10 days while the [¹⁶¹Tb]Tb-DO3A-non targeting IgG demonstrated a tumor doubling time of about 18 days. Interestingly, a significant antitumor effect was observed in mice treated with the [¹⁶¹Tb]Tb-DO3A-22B (p<0.001). For example, the tumor doubling time for the mice treated with 100 μCi [¹⁶¹Tb]Tb-DO3A-22B was about 28 days. Increasing the dose 2 fold increased the tumor doubling time to about 30 days. These results demonstrate the therapeutic efficacy of [¹⁶¹Tb]Tb-DO3A-22B in PDAC cancers.

Monitoring of the body weight was assessed to observe acute toxicity of [¹⁶¹Tb]Tb-DO3A-22B (FIG. 30B). No significant acute toxicity associated with drug administration was observed for either immunoconjugate. This was demonstrated by no significant decrease in the body weight (as measured by body weight loss of greater than 20%) for any of the treatment groups observed. The probability of survival was assessed for mice not treated (control), mice treated with [¹⁶¹Tb]Tb-DO3A-22B, and mice treated with [¹⁶¹Tb]Tb-DO3A-non targeting IgG control (FIG. 30C). Survival analysis of the [¹⁶¹Tb]Tb-DO3A-22B 200 μCi group demonstrated a survival benefit over the control group (p=0.0049, log-rank test). The median survival times for the [¹⁶¹Tb]Tb-DO3A-22B 200 μCi group, [¹⁶¹Tb]Tb-DO3A-22B 100 μCi group, [¹⁶¹Tb]Tb-DO3A-non targeting IgG group, and control group were 70, 39, 35.5, 35, and 32 days, respectively. These results highlight the therapeutic potential of [¹⁶¹Tb]Tb-DO3A-22B in this tumor model at minimum effective dose ranges.

### Example 14. Exemplary Embodiments

Embodiment 1 comprises an isolated antibody or antibody fragment, comprising: a heavy chain variable region comprising (i) a CDRH1 comprising SEQ ID NO: 3; (ii) a CDRH2 comprising SEQ ID NO: 4; and (iii) a CDRH3 comprising SEQ ID NO: 5; and a light chain variable region comprising (i) a CDRL1 comprising SEQ ID NO: 6; (ii) a CDRL2 comprising SEQ ID NO: 7; and (iii) a CDRL3 comprising SEQ ID NO: 8.

Embodiment 2 comprises the isolated antibody or antibody fragment of embodiment 1, comprising: a heavy chain variable region (VH) having at least 90% identity to SEQ ID NO: 1; and a light chain variable region (VL) having at least 90% identity to SEQ ID NO: 2.

Embodiment 3 comprises the isolated antibody or antibody fragment of any one of embodiments 1 or 2, wherein the antibody or antibody fragment comprises a light chain variable sequence as set forth in SEQ ID NO: 2.

Embodiment 4 comprises the isolated antibody or antibody fragment of any one of embodiments 1 to 3, wherein the antibody or antibody fragment comprises a heavy chain variable sequence as set forth in SEQ ID NO: 1.

Embodiment 5 comprises the isolated antibody or antibody fragment of any one of Embodiments 1 to 4, wherein the antibody or antibody fragment comprises a heavy chain variable sequence as set forth in SEQ ID NO: 1 and a light chain variable sequence as set forth in SEQ ID NO: 2.

Embodiment 6 comprises the isolated antibody or antibody fragment of any one of Embodiments 1 to 5, wherein the antibody or antibody fragment is non-fucosylated.

Embodiment 7 comprises the isolated antibody or antibody fragment of any one of Embodiments 1 to 6, wherein the antibody fragment is a monovalent scFv (single chain fragment variable) antibody, divalent scFv, Fab fragment, F(ab′)2 fragment, F(ab′)3 fragment, Fv fragment, nanobody, or single chain antibody.

Embodiment 8 comprises the isolated antibody or antibody fragment of any one of Embodiments 1 to 7, wherein the antibody or antibody fragment is a chimeric antibody, bispecific antibody, trispecific or other multi-specific antibody, or BiTE.

Embodiment 9 comprises the isolated antibody or antibody fragment of any one of Embodiments 1 to 8, wherein the antibody is an IgG antibody or a recombinant IgG antibody or antibody fragment.

Embodiment 10 comprises the isolated antibody or antibody fragment of any one of Embodiments 1 to 9, wherein the antibody is conjugated or fused to an imaging agent, a cytotoxic agent, a metal, or a radioactive moiety.

Embodiment 11 comprises the isolated antibody or antibody fragment of Embodiment 10, wherein the antibody or antibody fragment is conjugated or fused to an imaging agent, and wherein the imaging agent is a fluorophore.

Embodiment 12 comprises the isolated antibody or antibody fragment of Embodiment 10, wherein the antibody or antibody fragment is conjugated or fused to a radioactive moiety, and wherein the radioactive moiety is selected from a group consisting of 161Tb, 225Ac, 161Tb/225Ac, 89Zr, 177Lu, 134Ce, 140Nd, 169Er, 134Ce/134La, and 140Nd/140Pr.

Embodiment 13 comprises the isolated antibody or antibody fragment of any one of Embodiments 1 to 9, wherein the antibody is an immune conjugate.

Embodiment 14 comprises the isolated antibody or antibody fragment of Embodiment 10, wherein the antibody is conjugated to flagellin or a flagellin derivative.

Embodiment 15 comprises the isolated antibody or antibody fragment of any one of Embodiment 1 to 9, wherein the antibody is an antibody-drug conjugate.

Embodiment 16 comprises the isolated antibody or antibody fragment of any one of Embodiment 1 to 15 and a pharmaceutically acceptable carrier.

Embodiment 17 comprises an isolated nucleic acid encoding the antibody heavy and/or light chain variable region of the antibody or antibody fragment of any one of Embodiments 1 to 9.

Embodiment 18 comprises an expression vector comprising the isolated nucleic acid of Embodiment 17.

Embodiment 19 comprises a hybridoma or engineered cell comprising a nucleic acid encoding the antibody or antibody fragment of any one of Embodiments 1 to 9.

Embodiment 20 comprises a hybridoma or engineered cell comprising the nucleic acid of Embodiment 17.

Embodiment 21 comprises a method of making an isolated antibody or antibody fragment, comprising culturing the hybridoma or engineered cell of Embodiment 19 or Embodiment 20 under conditions that allow expression of the antibody or antibody fragment and, optionally, isolating the antibody from the culture.

Embodiment 22 comprises a chimeric antigen receptor (CAR) protein comprising an antigen binding domain comprising a heavy chain variable region (VH) having at least 90% identity to SEQ ID NO: 1 and comprising a CDRH1 amino acid sequence comprising SEQ ID NO: 3, a CDRH2 amino acid sequence comprising SEQ ID NO: 4, and a CDRH3 amino acid sequence comprising SEQ ID NO: 5; and a light chain variable region (VL) having at least 90% identity to SEQ ID NO: 2 and comprising a CDRL1 amino acid sequence comprising SEQ ID NO: 6, a CDRL2 amino acid sequence comprising SEQ ID NO: 7, and a CDRL3 amino acid sequence comprising SEQ ID NO: 8.

Embodiment 23 comprises the CAR of Embodiment 22, wherein the antigen binding domain comprises a heavy chain variable region (VH) comprising CDRH1, CDRH2, and CDRH3 amino acid sequences derived from SEQ ID NO: 1; and a light chain variable region (VL) comprising CDRH1, CDRH2, and CDRH3 amino acid sequences derived from SEQ ID NO: 2.

Embodiment 24 comprises the CAR of Embodiment 22 or Embodiment 23, wherein the antigen binding domain comprises a heavy chain variable sequence having a sequence set forth in SEQ ID NO: 1 and a light chain variable sequence having a sequence set forth in SEQ ID NO: 2.

Embodiment 25 comprises the CAR of any one of Embodiments 22 to 24, wherein the antigen binding domain specifically binds syndecan 1 (SDC1).

Embodiment 26 comprises the CAR of any one of Embodiments 22 to 25, further comprising a hinge domain, a transmembrane domain, and an intracellular signaling domain.

Embodiment 27 comprises the CAR of claim 26, wherein the hinge domain is a CD8a hinge domain or an IgG4 hinge domain.

Embodiment 28 comprises the CAR of Embodiment 26, wherein the transmembrane domain is a CD8a transmembrane domain or a CD28 transmembrane domain.

Embodiment 29 comprises the CAR of Embodiment 26, wherein the intracellular signaling domain comprises a CD3z intracellular signaling domain.

Embodiment 30 comprises a nucleic acid molecule encoding the CAR of any one of Embodiments 22 to 29.

Embodiment 31 comprises the nucleic acid molecule of Embodiment 30, wherein the nucleic acid sequence encoding the CAR is operatively linked to an expression control sequence.

Embodiment 32 comprises an expression vector comprising the nucleic acid molecule of Embodiment 30 or Embodiment 31.

Embodiment 33 comprises an engineered cell comprising the nucleic acid molecule of any one of Embodiments 30 or 31.

Embodiment 34 comprises the cell of Embodiment 33, wherein the cell is a T cell. Embodiment 35 comprises the cell of Embodiment 33, wherein the cell is an NK cell.

Embodiment 36 comprises the cell of Embodiment 33, wherein the nucleic acid is integrated into a genome of the cell.

Embodiment 37 comprises the cell of any one of Embodiments 33 to 36, wherein the cell is a human cell.

Embodiment 38 comprises a pharmaceutical composition comprising a population of cells in accordance with any one of Embodiments 33 to 37 and a pharmaceutically acceptable carrier.

Embodiment 39 comprises a method of treating cancer in a patient, comprising administering to the patient an anti-tumor effective amount of the pharmaceutical composition of Embodiment 15 or Embodiment 38.

Embodiment 40 comprises the method of Embodiment 39, wherein the composition comprises a population of cells, and wherein the cells are allogeneic cells.

Embodiment 41 comprises the method of Embodiment 39, wherein the composition comprises a population of cells, and wherein the cells are autologous cells.

Embodiment 42 comprises the method of Embodiment 39, wherein the composition comprises a population of cells, and wherein the cells are HLA matched to the patient.

Embodiment 43 comprises the method of Embodiment 39, wherein the composition comprises an isolated antibody or antibody fragment thereof conjugated to a therapeutic agent.

Embodiment 44 comprises the method of Embodiment 43, wherein the therapeutic agent is at least one of a cytotoxic agent, a chemotherapeutic agent, or an immunosuppressive agent.

Embodiment 45 comprises the method of any one of Embodiments 43 or 44, wherein the therapeutic agent is a moiety that specifically binds to an immune cell.

Embodiment 46 comprises the method of Embodiment 45, wherein the immune cell is a T cell.

Embodiment 47 comprises the method of Embodiment 45, wherein the immune cell is a natural killer cell.

Embodiment 48 comprises the method of any one of Embodiments 39 to 47, wherein the cancer has been determined to express an elevated level of SDC1 relative to a healthy tissue.

Embodiment 49 comprises the method of any one of Embodiments 39 to 48, wherein the cancer is a pancreatic cancer, a colorectal cancer, or a non-small cell lung cancer.

Embodiment 50 comprises the method of any one of Embodiments 39 to 49, wherein the administration of the pharmaceutical composition reduces macropinocytosis in the patient.

Embodiment 51 comprises the method of any one of Embodiments 39 to 50, wherein the patient has previously failed to respond to an immune checkpoint inhibitor.

Embodiment 52 comprises the method of Embodiment 51, wherein the patient has relapsed.

Embodiment 53 comprises the method of any one of Embodiments 39 to 52, further comprising administering at least a second anti-cancer therapy.

Embodiment 54 comprises the method of Embodiment 53, wherein the second anti-cancer therapy is a chemotherapy, molecular targeted therapy, immunotherapy, radiotherapy, radioimmunotherapy, phototherapy, gene therapy, surgery, hormonal therapy, epigenetic modulation, anti-angiogenic therapy or cytokine therapy.

Embodiment 55 comprises the method of Embodiment 53, wherein the second anti-cancer therapy is selected from a group consisting of a PD1 antibody, a 4-1BB antibody, gemcitabine, AMG510, and MRTX1133.

Embodiment 56 comprises a method of detecting the presence of SDC1 in a biological sample comprising: (a) contacting a biological sample with the isolated antibody or antibody fragment thereof of any one of Embodiments 1 to 15, and (b) detecting an amount of binding of the isolated antibody or antibody fragment thereof as a determination of the presence of SDC1 in the biological sample.

Embodiment 57 comprises the method of Embodiment 56, wherein the biological sample comprises cancer cells.

Embodiment 58 comprises the method of Embodiment 56, wherein the biological sample comprises a sample from a tumor from a patient.

Embodiment 59 comprises a method of imaging a tumor in a patient with an SDC1 expressing cancer, the method comprising: (a) administering to the patient an isolated antibody or antibody fragment thereof of any one of Embodiments 1 to 9 conjugated to an imaging label, and (b) detecting the imaging label in the patient to obtain an image of the tumor.

Embodiment 60 comprises a method of monitoring response of a patient with an SDC1 expressing cancer to cancer therapy, comprising: (a) administering to the patient the isolated antibody or antibody fragment thereof of any one of Embodiments 1 to 9 conjugated to an imaging label at a first time point before the patient receives cancer therapy; (b) detecting the imaging label in the patient to obtain a first image of a tumor: (c) administering to the patient an isolated antibody or antibody fragment thereof of any one of Embodiments 1 to 9 conjugated to an imaging label at a second time point after the patient receives cancer therapy; (d) detecting the imaging label in the patient to obtain a second image of the tumor; and (e) comparing the first image to the second image to determine whether a change in tumor size has occurred.

Embodiment 61 comprises the method of Embodiment 60, wherein steps (c) to (e) are repeated at a third time point after the patient receives cancer therapy.

Embodiment 62 comprises the method of any one of Embodiments 60 or 61, wherein the imaging label comprises a radioisotope, a bioluminescent label, a chemiluminescent label, or a paramagnetic compound.

Embodiment 63 comprises a method of assessing the likelihood of responsiveness of a patient with cancer to treatment with an SDC1 targeted therapy, comprising: (a) measuring in a tumor sample from a patient an amount of expression of SDC1; and (b) determining if the patient has a cancer characterized as having a high level of SDC1 expression.

Embodiment 64 comprises the method of Embodiment 63, wherein the amount of SDC1 expression in the tumor sample is measured using the isolated antibody or antibody fragment thereof of any one of Embodiments 1 to 9.

Embodiment 64 comprises the method of Embodiment 63 or Embodiment 64, wherein the SDC1 targeted therapy comprises administration of the pharmaceutical composition of Embodiment 16 or Embodiment 38.

Embodiment 65 comprises a theranostic pair comprising: a first radiolabeled antibody comprising a syndecan 1 (SDC1) antibody or antibody fragment thereof conjugated to a first chelating agent and a first radioactive moiety that is an imaging agent; and a second radiolabeled antibody comprising the SDC1 antibody or antibody fragment thereof conjugated to a second chelating agent and a second radioactive moiety that is a therapeutic agent.

Embodiment 66 comprises the theranostic pair of Embodiment 65, wherein the SDC1 antibody or antibody fragment comprises: a. a heavy chain variable region comprising: (j) a CDRH1 comprising SEQ ID NO: 3; (ii) a CDRH2 comprising SEQ ID NO: 4; and (iii) a CDRH3 comprising SEQ ID NO: 5; and b. a light chain variable region comprising: (iv) a CDRL1 comprising SEQ ID NO: 6: (v) a CDRL2 comprising SEQ ID NO: 7; and (vi) a CDRL3 comprising SEQ ID NO: 8.

Embodiment 67 comprises the theranostic pair of any one of Embodiments 65 or 66, wherein the first radioactive moiety is selected from a group consisting of 89Zr, 131I, 125I, 123I, 111I, 99mTc, 90Y, 186Re, 188Re, 32P, 153Sm, 67Ga, 201Tl, 77Br, or 18F.

Embodiment 68 comprises the theranostic pair of any one of claims 65 to 67, wherein the first radioactive moiety is 89Zr.

Embodiment 69 comprises the theranostic pair of any one of Embodiments 65 to 68, wherein the second radioactive moiety is selected from a group consisting of 161Tb, 225Ac, 161Tb/225Ac, 177Lu, 134Ce, 140Nd, 169Er, 134Ce/134La, and 140Nd/140Pr.

Embodiment 70 comprises the theranostic pair of any one of Embodiments 65 to 69, wherein the second radioactive moiety is 161Tb.

Embodiment 71 comprises the theranostic pair of any one of Embodiments 65 to 70, wherein the SDC1 antibody or antibody fragment comprises a heavy chain variable region (VH) having at least 90% identity to SEQ ID NO: 1; and a light chain variable region (VL) having at least 90% identity to SEQ ID NO: 2.

Embodiment 72 comprises the theranostic pair of any one of Embodiments 65 to 71, wherein the SDC1 antibody or antibody fragment comprises a heavy chain variable region (VH) comprising SEQ ID NO: 1; and a light chain variable region (VL) comprising SEQ ID NO: 2.

Embodiment 73 comprises the theranostic pair of any one of Embodiments 65 to 72, wherein the chelating agent comprises deferoxamine (DFO), 1,4,7,10-tetraazacyclododecane (DO3A), or diethylenetriaminepentaacetic acid (DTPA).

Embodiment 74 comprises the theranostic pair of any one of Embodiments 65 to 73, wherein the first radiolabeled antibody comprises the SDC1 antibody or antibody fragment thereof conjugated to DFO and 89Zr, and wherein the second radiolabeled antibody comprises the SDC1 antibody or antibody fragment thereof conjugated to DO3A and 161Tb.

Embodiment 75 comprises the theranostic pair of any one of Embodiments 65 to 74, further comprising a third radiolabeled antibody comprising the SDC1 antibody or antibody fragment thereof conjugated to a third chelating agent and a third radioactive moiety that is a therapeutic agent, wherein the third radioactive moiety is selected from a group consisting of 161Tb, 225Ac, 161Tb/225Ac, 177Lu, 134Ce, 140Nd, 169Er, 134Ce/134La, and 140Nd/140Pr.

Embodiment 76 comprises a method of treating cancer in a subject, comprising: (a) detecting the presence of a tumor in the subject by administering a first radiolabeled antibody comprising a syndecan 1 (SDC1) antibody or antibody fragment thereof conjugated to a first chelating agent and a first radioactive moiety that is an imaging agent; and (b) treating the subject by administering a therapeutically effective amount of a second radiolabeled antibody comprising the SDC1 antibody or antibody fragment thereof conjugated to a second chelating agent and a second radioactive moiety that is a therapeutic agent.

Embodiment 77 comprises the method of Embodiment 76, wherein the SDC1 antibody or antibody fragment comprises: a. a heavy chain variable region comprising: (k) a CDRH1 comprising SEQ ID NO: 3; (ii) a CDRH2 comprising SEQ ID NO: 4; and (iii) a CDRH3 comprising SEQ ID NO: 5; and b. a light chain variable region comprising (vii) a CDRL1 comprising SEQ ID NO: 6: (viii) a CDRL2 comprising SEQ ID NO: 7; and (ix) a CDRL3 comprising SEQ ID NO: 8.

Embodiment 78 comprises the method of any one of Embodiments 76 or 77, wherein the first radioactive moiety is selected from a group consisting of 89Zr, 131I, 125I, 123I, 111I, 99mTc, 90Y, 186Re, 188Re, 32P, 153Sm, 67Ga, 201Tl, 77Br, or 18F.

Embodiment 79 comprises the method of any one of Embodiments 76 to 78, wherein the first radioactive moiety is 89Zr.

Embodiment 80 comprises the method of any one of Embodiments 76 to 79, wherein the second radioactive moiety is selected from a group consisting of 161Tb, 225Ac, 161Tb/225Ac, 177Lu, 134Ce, 140Nd, 169Er, 134Ce/134La, and 140Nd/140Pr.

Embodiment 81 comprises the method of any one of Embodiments 76 to 80, wherein the second radioactive moiety is 161Tb.

Embodiment 82 comprises the method of any one of Embodiments 76 to 81, wherein the SDC1 antibody or antibody fragment comprises a heavy chain variable region (VH) having at least 90% identity to SEQ ID NO: 1; and a light chain variable region (VL) having at least 90% identity to SEQ ID NO: 2.

Embodiment 83 comprises the method of any one of Embodiments 76 to 82, wherein the SDC1 antibody or antibody fragment comprises a heavy chain variable region (VH) comprising SEQ ID NO: 1; and a light chain variable region (VL) comprising SEQ ID NO: 2.

Embodiment 84 comprises the method of any one of Embodiments 76 to 83, wherein the chelating agent comprises deferoxamine (DFO), 1,4,7,10-tetraazacyclododecane (DO3A), or diethylenetriaminepentaacetic acid (DTPA).

Embodiment 85 comprises the method of any one of Embodiments 76 to 84, wherein the first radiolabeled antibody comprises the SDC1 antibody or antibody fragment thereof conjugated to DFO and 89Zr, and wherein the second radiolabeled antibody comprises the SDC1 antibody or antibody fragment thereof conjugated to DO3A and 161Tb.

Embodiment 86 comprises the method of any one of Embodiments 76 to 85, wherein the second radiolabeled antibody is administered at a concentration of about 100. Ci to 300□Ci.

Embodiment 87 comprises the method of any one of Embodiments 76 to 86, wherein the cancer is a pancreatic cancer, a colorectal cancer, or a non-small cell lung cancer.

Embodiment 88 comprises the method of any one of Embodiments 76 to 87, wherein the subject has relapsed.

Embodiment 89 comprises the method of any one of Embodiments 76 to 88, further comprising administering at least a second anti-cancer therapy.

Embodiment 90 comprises the method of Embodiment 89, wherein the second anti-cancer therapy is a chemotherapy, molecular targeted therapy, immunotherapy, radiotherapy, radioimmunotherapy, phototherapy, gene therapy, surgery, hormonal therapy, epigenetic modulation, anti-angiogenic therapy or cytokine therapy.

Embodiment 91 comprises the method of Embodiment 89, wherein the method further comprises administering at least a third radiolabeled antibody comprising the SDC1 antibody or antibody fragment thereof conjugated to a third chelating agent and a third radioactive moiety that is a therapeutic agent, wherein the third radioactive moiety is selected from a group consisting of 225Ac, 161Tb/225Ac, 177Lu, 134Ce, 140Nd, 169Er, 134Ce/134La, and 140Nd/140Pr.

Embodiment 92 comprises a method of monitoring response of a subject with an SDC1-expressing cancer to an anti-cancer therapy, comprising: (a) detecting the presence of a tumor in the subject at a first time point by administering a first radiolabeled antibody comprising a syndecan 1 (SDC1) antibody or antibody fragment thereof conjugated to a first chelating agent and a first radioactive moiety that is an imaging agent and obtaining a first image of the tumor; and (b) treating the subject by administering a therapeutically effective amount of a second radiolabeled antibody comprising the SDC1 antibody or antibody fragment thereof conjugated to a second chelating agent and a second radioactive moiety that is a therapeutic agent: (c) detecting the presence or absence of the tumor in the subject at a second time point after the subject has been treated with cancer therapy by administering the first radiolabeled antibody and obtaining a second image of the tumor; and (d) comparing the first image to the second image to determine whether a change in tumor size has occurred.

Embodiment 93 comprises the method of Embodiment 92, wherein the SDC1 antibody comprises: (1) a heavy chain variable region comprising: (A) a CDRH1 comprising SEQ ID NO: 3: (B) a CDRH2 comprising SEQ ID NO: 4; and (C) a CDRH3 comprising SEQ ID NO: 5; and (2) a light chain variable region comprising (A) a CDRL1 comprising SEQ ID NO: 6: (B) a CDRL2 comprising SEQ ID NO: 7; and (C) a CDRL3 comprising SEQ ID NO: 8.

Embodiment 94 comprises the method of any one of Embodiments 92 or 93, wherein steps (c) and (d) are repeated at a third time point after the subject receives anti-cancer therapy.

Embodiment 95 comprises the method of any one of Embodiments 92 to 94, wherein the anti-cancer therapy is a chemotherapy, molecular targeted therapy, immunotherapy, radiotherapy, radioimmunotherapy, phototherapy, gene therapy, surgery, hormonal therapy, epigenetic modulation, anti-angiogenic therapy or cytokine therapy.

Embodiment 96 comprises the method of any one of Embodiments 92 to 95, wherein the anti-cancer therapy comprises administering at least a third radiolabeled antibody comprising the SDC1 antibody or antibody fragment thereof conjugated to a third chelating agent and a third radioactive moiety that is a therapeutic agent, wherein the third radioactive moiety is selected from a group consisting of 225Ac, 161Tb/225Ac, 177Lu, 134Ce, 140Nd, 169Er, 134Ce/134La, and 140Nd/140Pr.

Embodiment 97 comprises the method of any one of Embodiments 92 to 96, wherein the first radioactive moiety is selected from a group consisting of 89Zr, 131I, 125I, 123I, 111I, 99mTc, 90Y, 186Re, 188Re, 32P, 153Sm, 67Ga, 201Tl, 77Br, or 18F.

Embodiment 98 comprises the method of any one of Embodiments 92 to 97, wherein the first radioactive moiety is 89Zr.

Embodiment 99 comprises the method of any one of Embodiments 92 to 98, wherein the second radioactive moiety is selected from a group consisting of 161Tb, 225Ac, 161Tb/225Ac, 177Lu, 134Ce, 140Nd, 169Er, 134Ce/134La, and 140Nd/140Pr.

Embodiment 100 comprises the method of any one of Embodiments 92 to 99, wherein the second radioactive moiety is 161Tb.

Embodiment 101 comprises the method of any one of Embodiments 92 to 100, wherein the SDC1 antibody or antibody fragment comprises a heavy chain variable region (VH) having at least 90% identity to SEQ ID NO: 1; and a light chain variable region (VL) having at least 90% identity to SEQ ID NO: 2.

Embodiment 102 comprises the method of any one of Embodiments 92 to 101, wherein the SDC1 antibody or antibody fragment comprises a heavy chain variable region (VH) comprising SEQ ID NO: 1; and a light chain variable region (VL) comprising SEQ ID NO: 2.

Embodiment 103 comprises the method of any one of Embodiments 92 to 102, wherein the chelating agent comprises deferoxamine (DFO), 1,4,7,10-tetraazacyclododecane (DO3A), or diethylenetriaminepentaacetic acid (DTPA).

Embodiment 104 comprises the method of any one of Embodiments 92 to 103, wherein the first radiolabeled antibody comprises the SDC1 antibody or antibody fragment thereof conjugated to DFO and 89Zr, and wherein the second radiolabeled antibody comprises the SDC1 antibody or antibody fragment thereof conjugated to DO3A and 161Tb.

SEQUENCES

| SEQ | | |
|---|---|---|
| ID | | |
| NO | Sequence | Description |
| 1 | QVQLQQPGAELARPGAAVKLSCKASGYTFTNYWMNWVKQRPGQ | 22B-VH |
| | GLEWIGMIDPSDNKTLYNPMFKDKATLTVDKSSSTAYMQLSSL | |
| | TSEDSAVYYCARRGFAYWGQGTLVTVSA | |
| 2 | MTQTPSSLSASLGDTITITCHASQNINVWLSWYQQKPGNIPKVLI | 22B-VL |
| | YKASNLHTGVPSRFSGSGSGTGFTLTISSLQPEDIATYYCQQGQS | |
| | YPLTFGGGTKLEIK | |
| 3 | NYWMN | 22B |
| | | CDRVH1 |
| 4 | MIDPSDNK | 22B |
| | | CDRVH2 |
| 5 | RGFAY | 22B |
| | | CDRVH3 |
| 6 | HASQNINVWLS | 22B |
| | | CDRVL1 |
| 7 | KASNLHT | 22B |
| | | CDRVL2 |
| 8 | QQGQSYPLT | 22B |
| | | CDRVL3 |
| 9 | CAGGTCCAACTGCAGCAGCCTGGGGCTGAGCTGGCGAGGCCT | 22B VH |
| | GGGGCTGCAGTGAAGCTGTCCTGCAAGGCTTCTGGCTACACCT | nucleotide |
| | TCACCAACTACTGGATGAACTGGGTGAAGCAGAGGCCTGGAC | |
| | AAGGCCTTGAATGGATTGGTATGATTGATCCTTCAGACAATAA | |
| | AACTCTCTACAATCCAATGTTCAAGGACAAGGCCACATTGACT | |
| | GTAGACAAATCCTCCAGCACAGCCTACATGCAGCTCAGCAGT | |
| | CTGACATCTGAGGACTCTGCGGTCTATTACTGTGCAAGACGAG | |
| | GCTTTGCTTACTGGGGCCAAGGGACTCTGGTCACTGTAAG | |
| 10 | ATGACCCAGACTCCATCCAGTCTGTCTGCATCCCTTGGAGACA | 22B VL |
| | CAATTACCATCACTTGCCATGCCAGTCAGAACATTAATGTTTG | nucleotide |
| | GTTAAGCTGGTACCAGCAGAAACCAGGAAATATTCCTAAAGT | |
| | ATTGATCTATAAGGCCTCCAATTTGCACACAGGCGTCCCATCA | |
| | AGGTTTAGTGGCAGTGGATCTGGAACAGGTTTCACATTAACCA | |
| | TCAGCAGCCTGCAGCCTGAAGACATTGCCACTTACTACTGTCA | |
| | ACAGGGTCAAAGTTATCCTCTGACGTTTGGTGGAGGCACCAA | |
| | ACTGGAAATCAAA | |
| 11 | VQLQESGPELVKPGASVKMSCKASGYTFTDYYMNWVKQSHGK | 226-10-VH |
| | SLEWIGDINPYNGGTSYNQKFKGKATLTVDKSSRTAYMQLNSLT | |
| | SEDSAVYYCARGGSPQWGQGTTLTVSS | |
| 12 | DIVMTQTPLTLSVTIGQPASISCKSSQSLLHSDGKTYLNWLLQRPG | 226-10-VL |
| | QSPKRLIYLVSKLDSGVPDRFTGSGSGTDFTLKISRVEAEDLGVY | |
| | YCWQGTHFPWTFGGGTKLEIK | |
| 13 | MGWSCIILFLVATATGVHSQVQLQQPGAELVRPGAAVKLSCKAS | 27-VH |
| | GYTFTSYWMNWVKQRPGQGLEWIGMIDPSDSKTHYNQMFKDK | |
| | ATLTVDKSSSTAYMQLSSLTFEDSAVYYCARRGFPYWGQGTLVT | |
| | VSA | |
| 14 | MRVLAELLGLLLFCFLGVRCDIQMNQSPSSLSASLGDTITITCHAS | 27-VL |
| | QNINVWLSWYQQKPGNIPKVLIYKASNLHTGVPSRFSGSGSGTGF | |
| | TLTISSLQPEDIATYYCQQGQSYPLTFGGGTKLEIK | |
| 15 | DYKDDDDK | FLAG |
| 16 | HHHHHH | polyhistidine |
| | | (6-His) |
| 17 | YPYDVPDYA | hemagglutinin |
| | | (HA) |
| 18 | TTTPAPRPPTPAPTIASQPLSLRPEACRPAAGGAVHTRGLDFACD | CD8α hinge |
| 19 | IYIWAPLAGTCGVLLLSLVITLYC | CD8α TM |
| | | domain |
| 20 | KRGRKKLLYIFKQPFMRPVQTTQEEDGCSCRFPEEEEGGCEL | 4-1BB |
| | | signaling |
| | | domain |
| 21 | GGGGSGGGGSGGGGS | scFv linker |
| 22 | DITLSQ | SDC1 |
| | | epitope |
| 23 | MRRAALWLWLCALALSLQPALPQIVATNLPPEDQDGSGDDSDNF | SDC1 human |
| | SGSGAGALQDITLSQQTPSTWKDTQLLTAIPTSPEPTGLEATAA | |
| | STSTLPAGEGPKEGEAVVLPEVEPGLTAREQEATPRPRETTQLP | |
| | TTHLASTTTATTAQEPATSHPHRDMQPGHHETSTPAGPSQADLH | |
| | TPHTEDGGPSATERAAEDGASSQLPAAEGSGEQDFTFETSGENT | |
| | AVVAVEPDRRNQSPVDQGATGASQGLLDRKEVLGGVIAGGLVGL | |
| | IFAVCLVGFMLYRMKKKDEGSYSLEEPKQANGGAYQKPTKQEEF | |
| | YA | |
| 24 | MRRAALWLWLCALALRLQPALPQIVAVNVPPEDQDGSGDDSDNF | SDC1 mouse |
| | SGSGTGALPDTLSRQTPSTWKDVWLLTATPTAPEPTSSNTETAF | |
| | TSVLPAGEKPEEGEPVLHVEAEPGFTARDKEKEVTTRPRETVQL | |
| | PITQRASTVRVTTAQAAVTSHPHGGMQPGLHETSAPTAPGQPDH | |
| | QPPRVEGGGTSVIKEVVEDGTANQLPAGEGSGEQDFTFETSGEN | |
| | TAVAAVEPGLRNQPPVDEGATGASQSLLDRKEVLGGVIAGGLVG | |
| | LIFAVCLVAFMLYRMKKKDEGSYSLEEPKQANGGAYQKPTKQEE | |
| | FYA | |
| 25 | DFTF | SDC1 |
| | | epitope |

What is claimed:

1. An isolated antibody or antibody fragment, comprising:
a. a heavy chain variable region comprising
(i) a CDRH1 comprising SEQ ID NO: 3;
(ii) a CDRH2 comprising SEQ ID NO: 4; and
(iii) a CDRH3 comprising SEQ ID NO: 5; and
b. a light chain variable region comprising
(i) a CDRL1 comprising SEQ ID NO: 6;
(ii) a CDRL2 comprising SEQ ID NO: 7; and
(iii) a CDRL3 comprising SEQ ID NO: 8.

2. The isolated antibody or antibody fragment of claim 1, comprising:
a heavy chain variable region (VH) having at least 90% identity to SEQ ID NO: 1; and
a light chain variable region (VL) having at least 90% identity to SEQ ID NO: 2.

3. The isolated antibody or antibody fragment of claim 1, wherein the antibody or antibody fragment comprises a light chain variable sequence as set forth in SEQ ID NO: 2 and a heavy chain variable sequence as set forth in SEQ ID NO: 1.

4. The isolated antibody or antibody fragment of claim 1, wherein the antibody or antibody fragment is non-fucosylated.

5. The isolated antibody or antibody fragment of claim 1, wherein the antibody fragment is a monovalent scFv (single chain fragment variable) antibody, divalent scFv, Fab fragment, F(ab′)₂ fragment, F(ab′)₃ fragment, Fv fragment, nanobody, or single chain antibody.

6. The isolated antibody or antibody fragment of claim 1, wherein the antibody or antibody fragment is conjugated or fused to an imaging agent, and wherein the imaging agent is a fluorophore.

7. The isolated antibody or antibody fragment of claim 1, wherein the antibody or antibody fragment is conjugated or fused to a radioactive moiety, and wherein the radioactive moiety is selected from a group consisting of ¹⁶¹Tb, ²²⁵Ac, ¹⁶¹Tb/²²⁵Ac, ⁸⁹Zr, ¹⁷⁷Lu, ¹³⁴Ce, ¹⁴⁰Nd, ¹⁶⁹Er, ¹³⁴Ce/¹³⁴La, and ¹⁴⁰Nd/¹⁴⁰Pr.

8. The isolated antibody or antibody fragment of claim 1, wherein the antibody is an immune conjugate or an antibody-drug conjugate.

9. A pharmaceutical composition comprising the isolated antibody or antibody fragment of claim 1 and a pharmaceutically acceptable carrier.

10. An isolated nucleic acid encoding the antibody heavy and/or light chain variable region of the antibody or antibody fragment of claim 1.

11. A hybridoma or engineered cell comprising the nucleic acid of claim 10.

12. A method of treating cancer in a patient, comprising administering to the patient an anti-tumor effective amount of the pharmaceutical composition of claim 9.

13. The method of claim 12, wherein the cancer is a pancreatic cancer, a colorectal cancer, or a non-small cell lung cancer.

14. A chimeric antigen receptor (CAR) protein comprising:
an antigen binding domain comprising a heavy chain variable region (VH) having at least 90% identity to SEQ ID NO: 1 and comprising a CDRH1 amino acid sequence comprising SEQ ID NO: 3, a CDRH2 amino acid sequence comprising SEQ ID NO: 4, and a CDRH3 amino acid sequence comprising SEQ ID NO: 5; and
a light chain variable region (VL) having at least 90% identity to SEQ ID NO: 2 and comprising a CDRL1 amino acid sequence comprising SEQ ID NO: 6, a CDRL2 amino acid sequence comprising SEQ ID NO: 7, and a CDRL3 amino acid sequence comprising SEQ ID NO: 8;
a transmembrane domain; and
a signaling domain.

15. An engineered cell comprising the CAR of claim 14.

16. A method of treating cancer in a patient, comprising administering to the patient an anti-tumor effective amount of a population of the cells of claim 15 and a pharmaceutically acceptable carrier.

17. A method of detecting the presence of SDC1 in a biological sample comprising:
(a) contacting a biological sample with the isolated antibody or antibody fragment thereof of claim 1, and
(b) detecting an amount of binding of the isolated antibody or antibody fragment thereof as a determination of the presence of SDC1 in the biological sample.

18. The method of claim 17, wherein the biological sample comprises cancer cells.

19. The method of claim 17, wherein the biological sample comprises a sample from a tumor from a patient.

20. A theranostic pair comprising:
a first radiolabeled antibody comprising a syndecan 1 (SDC1) antibody or antibody fragment thereof conjugated to a first chelating agent and a first radioactive moiety that is an imaging agent; and
a second radiolabeled antibody comprising the SDC1 antibody or antibody fragment thereof conjugated to a second chelating agent and a second radioactive moiety that is a therapeutic agent;
wherein the SDC1 antibody or antibody fragment comprises:
a. a heavy chain variable region comprising:
(j) a CDRH1 comprising SEQ ID NO: 3:
(ii) a CDRH2 comprising SEQ ID NO: 4; and
(iii) a CDRH3 comprising SEQ ID NO: 5; and
b. a light chain variable region comprising
(iv) a CDRL1 comprising SEQ ID NO: 6;
(v) a CDRL2 comprising SEQ ID NO: 7; and
(vi) a CDRL3 comprising SEQ ID NO: 8.

21. The theranostic pair of claim 20, wherein the first radioactive moiety is selected from a group consisting of ⁸⁹Zr, ¹³¹I, ¹²⁵I, ¹²³I, ¹¹¹I, ^{99m}Tc, ⁹⁰Y, ¹⁸⁶Re, ¹⁸⁸Re, ³²P, ¹⁵³Sm, ⁶⁷Ga, ²⁰¹Tl, ⁷⁷Br, or ¹⁸F.

22. The theranostic pair of claim 20, wherein the second radioactive moiety is selected from a group consisting of ¹⁶¹Tb, ²²⁵Ac, ¹⁶¹Tb/²²⁵Ac, ¹⁷⁷Lu, ¹³⁴Ce, ¹⁴⁰Nd, ¹⁶⁹Er, ¹³⁴Ce/¹³⁴La, and ¹⁴⁰Nd/¹⁴⁰Pr.

23. The theranostic pair of claim 20, wherein the first radiolabeled antibody comprises the SDC1 antibody or antibody fragment thereof conjugated to DFO and 89Zr, and wherein the second radiolabeled antibody comprises the SDC1 antibody or antibody fragment thereof conjugated to DO3A and ¹⁶¹Tb.

24. A method of treating cancer in a subject using the theranostic pair of claim 20, comprising:
(a) detecting the presence of a tumor in the subject by administering the first radiolabeled antibody; and
(b) treating the subject by administering a therapeutically effective amount of the second radiolabeled antibody.

25. The method of claim 24, wherein the first radiolabeled antibody comprises the SDC1 antibody or antibody fragment thereof conjugated to DFO and 89Zr, and wherein the second radiolabeled antibody comprises the SDC1 antibody or antibody fragment thereof conjugated to DO3A and ¹⁶¹Tb.

26. The method of claim 24, wherein the second radiolabeled antibody is administered at a concentration of about 100 μCi to 300 μCi.

27. The method of claim 24, wherein the cancer is a pancreatic cancer, a colorectal cancer, or a non-small cell lung cancer.
